(19) United States
(12) Patent Application Publication
(10) Pub. No.: US 2006/0122105 A1
(43) Pub. Date: Jun. 8, 2006

(54) Method of improing the growth performance of an animal

(76) Inventors: Alan David Greve Strom, Highton (AU); Aleta Gai Knowles, Enmore (AU); Alan Husband, McMahons Point (AU)

Correspondence Address: MCKENNA LONG & ALDRIDGE LLP, 1900 K STREET, NW, WASHINGTON, DC, 20006, US

(21) Appl. No.: 10/515,508

(22) PCT Filed: May 24, 2002
(86) PCT No.: PCT/AU02/00659

§ 371 Date: Aug. 5, 2005

(30) Foreign Application Priority Data
May 25, 2001 (AU) PR 5278

(51) Int. Cl. A61K 38/17; A61K 31/60; A61K 31/498; A61K 31/192

(52) U.S. Cl. 514/12; 514/165; 514/249; 514/570; 514/567

(57) ABSTRACT

The invention broadly relates to a method of improving the growth performance of an animal. In particular the present invention relates to a method of improving the growth performance of an animal comprising the step of administering to an animal in need thereof a growth promoting amount of one or more anti-inflammatory agents.

### FIELD OF THE INVENTION

The invention broadly relates to a method of improving the growth performance of an animal. In particular the present invention relates to a method of improving the growth performance of an animal comprising the step of administering to an animal in need thereof a growth promoting amount of one or more anti-inflammatory agents.

### BACKGROUND OF THE INVENTION

In the agricultural industries of many countries, commercial livestock rearing systems have become commonplace. Commercial animal husbandry techniques have been used in rearing poultry, pigs and cattle and have resulted in greatly increased production of food products derived from these animals.

The commercial raising of livestock requires maximisation of growth rate and feed conversion efficiency so as to reduce the unit cost of production. This requirement has led to the development and widespread use of so called “feed additives”.

Feed additives have two general purposes. One purpose is to enhance the performance of the animal in terms of increased growth rate and/or increased feed conversion efficiency in healthy and nutritionally unchallenged animals. The other purpose is to maintain the health of the animal during periods of trauma or “stress” that inevitably occur in the current practices of intensive rearing thereby keeping the animal disease free.

In the early 1950's, researchers unexpectedly discovered that an antibiotic ingredient in chicken mash was a “growth factor.” The finding drastically changed the livestock and poultry industries and was an economic boon for pharmaceutical companies. Feed animals are now raised under highly controlled conditions and receive specialised feed with a variety of growth promoting additives.

Routine antibiotic administration to animals has become almost universal since the discovery that the addition of small amounts of antibiotics such as penicillin, tetracycline and sulfamethasine, to animal feed increases the growth of pigs and cattle. In 1979, about 70% of the beef cattle and veal, 90% of the swine, and virtually 100% of broilers reared in the United States consumed antibiotics as part of their daily feed. This use, accounting for nearly 40% of antibiotics sold in the United States, is estimated to save consumers $3.5 billion a year in food costs.

Animals raised under modern conditions optimised for growth promotion receive rations containing high proportions of protein, usually in the form of soybean or cottonseed meal (meat and bone or blood meal are used extensively in Australia), and high percentages of grains such as corn or milo, a type of sorghum (wheat and barley in Australia) Feed additives which have been used include such hormones as diethyl-stilbesterol, which also increases the rate of weight gain, and tranquillisers (not used widely for pigs) that prevent the effects of the stress brought on by confinement conditions from causing disease or weight loss.

Cattle ordinarily require 5 kilograms of feed to produce 1 kilogram of weight gain. Under optimal growth promoting conditions, and with enriched feed, they gain 1 kilogram with only 3 kilograms of feed.

Although hormones and antibiotics have greatly increased the rate of growth of food animals, the use of such additives has not been without problems. One of the hormones that is commonly used as a growth stimulant, diethyl-stilbesterol or DES, has been shown to be a carcinogen and has been banned from further use in most countries.

When antibiotics are mixed in animal feed, the compounds are spread throughout the environment exposing microorganisms to the antibiotics. The constant exposure of the microorganisms to antibiotics puts biological pressure on the microorganisms to develop a resistance to the antibiotics. This can result in a microorganism that is resistant to antibiotics and causes especially severe and difficult to treat infections.

An antibiotic-resistant microorganism is potentially a serious pathogen because it is difficult to control. If the organism causes an infection in an animal or in man, the infection may not be controlled with conventional antibiotics. If the infection is serious, there may not be time to determine which antibiotics are effective against the infecting bacteria. The problem has been especially serious when antibiotic resistant organisms in meat are consumed by people who themselves take antibiotics for treatment of disease. Antibiotics inhibit many of the normal microorganisms in the respiratory and gastrointestinal tracts. This allows the resistant one to proliferate rapidly and produce more serious disease. The combination of antibiotic resistant organisms from food and ineffective antibiotic treatment of people has caused most of the deaths due to salmonella food poisoning reported in the United States in the past several years.

As a result of the increasing appearance of antibiotic resistant bacteria in feed lots and several serious epidemics caused by antibiotic resistant bacteria, there is increasing governmental pressure to ban the use of antibiotics in animal feed. In fact, the World Health Organisation and the Australian Government have specified the need to use environmentally friendly alternative methods to control infection. The imminent ban or withdrawal of various antibiotics from livestock feed and water is likely to (i) increase the incidence of infection in animals and consequently reduce growth performance (ii) further reduce the health, fertility and breeding performance of animals. Consequently, there is an immediate and increasing need for new, safe and effective growth stimulators of feed animals, as well as a reduction in disease by enhancing health.

Various attempts at promoting animal growth without the use of antibiotics has been employed many using elaborate and circuitous means. These have included subcutaneous implants of hormones or complex salts having cations being made from complexes (see, for example, U.S. Pat. No. 6,197,815; U.S. Pat. No. 3,991,750; U.S. Pat. No. 4,067,994). None of these attempts have proven to be simple or effective. Accordingly, there is still a need for a method of improving the growth performance of animals, which is not reliant on the use of antibiotics or elaborate methodology.

The applicant has now surprisingly found that the administration of anti-inflammatory agents, and in particular cytokine receptor antagonists such as interleukin (IL)-1ra, increases the growth performance of animals while decreasing the amount of antibiotics. The applicant also has evidence that a similar growth performance effect can be achieved by administering soluble cytokine receptors such as TNFα receptor, IL-6 receptor, IL-4 receptor and IL-8 receptor, or cytokine blocking factors such as TNF blocking factor (Bargetzki et al, Cancer Research 53: 4010-13 (1993); Engelmann et al, Journal of Biological Chemistry 264: 11974-80 (1989)) or TNF-alpha inhibitor (Engelmann et al, Journal of Biological Chemistry 265: 1531-6 (1990); Seckinger et al, European Journal of Immunology 20: 1167-74 (1990)).

While not wishing to be bound by any particular theory or hypothesis, the applicant considers that the increases in growth performance observed in animals that have been administered anti-inflammatory agents result from the interplay of four key effects. These are:

1). Anti-inflammatory effect per se;

2). Immunoenhancement effect;

3) Anti-parasitic and anti-microbial effect; and

4). Stress reduction.

Each of these effects, either singly or together, profoundly impact upon the health and welfare of animals, which in turn affects the growth performance of animals and thereby the meat quality. For example:

1). Anti-Inflammation

Chronic inflammation is often seen in livestock and relates to immune activation triggered by persistent infections and environmental stimuli. Inflammation plays an important role in the initiation of immune responses to infection, however, chronic immune activation, particularly by persistent infection or microbial load, can have deleterious effects on growth and development and can reduce the effectiveness of vaccination. Consequences of excessive immune activation include the production of inflammatory cytokines, fever, inappetence, amino acid resorption from muscle and redirection of nutrients away from meat production. Anti-inflammatory agents could reduce the pathology of chronic immune activation, for example, by reducing the effects of inflammatory cytokines such as IL-1, IL-6, TGF-β, IL-11, IL-18, IL-12, IL-17, LIF, IFN-γ IL-8, TNF-α and GM-CSF. Alternatively, by administering soluble cytokine receptors for these inflammatory cytokines ie IL-1 receptor, IL-8 receptor, TNF-α receptor, IL-6 receptor et al, excessive amounts of circulating inflammatory cytokines can be reduced. Cytokine receptor antagonists such as IL-1ra, IL-6ra or TNF-αra, which competitively inhibit the binding of these pro-inflammatory cytokines to their respective membrane-expressed receptors, can be used to ameliorate the action of these cytokines.

2). Immunoenhancement Effect

a). TH1/TH2 Immune Responses

The inflammatory response is inextricably tied to the body's immune system. Interplay occurs between immune cytokine regulatory networks and the other regulatory systems of the body. Immune responses to infections or antigens can acutely bias each other. The immune response can be generalised by the type of T cell response. A T helper 1 (TH1) type response is principally involved in cell mediated immunity, whilst a TH2 pattern of response is often associated with humoral immunity. TH1 and TH2 type T cell subsets have been implicated in the regulation of many immune responses defined by cytokine patterns. TH2 cells express the cytokines interleukin (IL)-4, IL-5, IL-10, and IL-13. IL-3 expression is common to both TH1 and TH2 T cells. Whereas, TH1 cells express IL-2, IFNγ, and TNFβ. These TH2 cytokines influence B cell development and augment humoral responses such as the secretion of antibodies. Both types of TH cells influence each other by the cytokines they secrete. For example, TH2 cytokines, such as IL-10, can suppress TH1 functions. Other cytokines can also influence TH1 or TH2 development such as TNFβ, known to down regulate TH1 responses.

It is known that when there is no invading antigen, the action of Th1 cells is considered harmful to the body. Anti-inflammatory agents suppress the production of IFN-γ in Th1 cells. Also anti-inflammatory agents suppress the overproduction of Th1 cells and therefore enhance the production of Th2 (antibody-secreting) cells because these cells cross-regulate one another. This means that by administering particular anti-inflammatory agents the amount of pro-inflammatory cytokines are suppressed. Alternatively, by administering soluble cytokine receptors, cytokine receptor antagonists, or cytokine inhibitory factors of cytokines like IL-1, IL-4, IL-8, GM-CSF, IL-6 or TNF-α the overproduction of cytokines by TH1 cells may be reduced.

b). Antibody Isotype Switching

Antibodies are required to eliminate or protect against infection. Mature B cells undergo the process of switching-antibody class after antigenic stimulation. TH cells through physical contact and cytokines, referred to as switch factors, regulate isotype switching. Some of the cytokines known to be involved in isotype switching, either alone or in combination, are IL-4, IL-5, TNFβ, IL-1, IL-2, IL-6, and IL-13. IL-4 and IL-5 synergise to enhance IgG1 responses. For example, optimal IgG1 responses also requires IL-2. IL-1 can enhance IgA production in the presence of IL-5. TNFβ induces IgA production.

c). Immune Dysfunction

The genetic potential for most production traits is predetermined by birth. Many factors (stress, disease, nutrition, immunity etc.) determine whether this potential is achieved. The level and type of antigen exposure influences and establishes a ‘bias’ of the immune system. Most immune responses are biased towards a type that promotes immunity against bacteria and viruses or a type that promotes immunity against many parasites. While the genotype of an animal can influence this bias, the early experience by the neonate to antigens and infections can set the immune reactivity towards one or other type. This bias is altered depending on subsequent antigen exposure. Breeding programmes based on selection for production traits has appeared to be at the expense and detriment of immune competence or reactivity. This change has been further exacerbated by the persistent use of antibiotic supplements to water and feed, which has presumably resulted in an altered genetic potential to mount effective-immune responses.

d). Mucosal Immunity

The most prevalent areas of infection in livestock are mucosal sites, primarily the gastro-intestinal tract and the lungs. Thus, the mucosal immune system is the first line of defence against pathogens and disease. Cytokines, notably IL-5, IL-4, IL-6 and IL-10, play a significant role in the regulation and efficacy of immune responses in the mucosa.

IL-5 and IL-6 act upon B-1 and B-2 subpopulations of lymphocytes in the mucosal immune system. Deficiencies in either the production of IL-5 or IL-6, or their receptors result in significantly impaired production of IgA, the antibody isotype responsible for protective responses in the mucosa. Similarly, IL-5, IL-6 and the chemokine MIP-1 alpha have the capacity to increase IgA responses to mucosal vaccines. IL-4 has an immunoregulatory role in mucosal tissues, primarily by enhancing TH2 responses, and thus, enhancing antibody production. IL-4 is considered essential to the development of mucosal immune responses in the lung, via the involvement of TH2 pathways. Both IL-4 and IL-5 operate in concert in the lung, with IL-4 committing naive T cells to a TH2 phenotype which upon subsequent activation secrete IL-5, resulting in eosinophil accumulation. Furthermore, IL-4 and IL-10 play a role in mucosal tolerance, and thus, help regulate and dampen allergic type responses in the gut and reduce the susceptibility of animals to chronic inflammatory conditions of the gut.

3). Anti-Parasitic and Anti-Microbial Effect

a). Anti-Parasitic Effect

Acquired immune responses against pathogens generally fall into one of two types, cell mediated (TH1) or antibody mediated (TH2), and this is controlled by cytokines. Cytokines involved in TH2 responses are attractive therapeutic targets, as they could protect against ectoparasites and gastrointestinal worms and suppress inflammation induced by TH1 cytokines. TH2 cytokines induce eosinophilia, IgE synthesis, and mucus production that enhance protection against worms and other gut parasites.

b). Anti-Microbial Effects

Microbial infections remain a world-wide problem in terms of economic impacts and health, despite advances in nutrition, vaccines, chemicals and antibiotics. The immune response to microbial pathogens incorporates two systems of recognition. The first line of defence is innate immunity and this is followed, if required, by the ensuing adaptive response (cell mediated and antibody responses). By decreasing the inflammation with anti-inflammatory agents including phenylbutazone, flunixin meglumine and ketoprofen or intravenous DMSO there is an improvement in the blood flow to the affected tissue, which in turn assists in the innate immunity to help overcome the infection. This process may be assisted by using vasodilation drugs such as acetylpromazine, phenoxybenzamine, isoxsuprine, pentoxifylline, aspirin and heparin. An alternative approach is to administer the soluble cytokine receptors of known inflammatory cytokines such as IL-1, TNF-α, IL-6 and IL-8.

4). Stress Reduction

Many conditions within a commercial environment contribute to a reduction in feed intake, growth rate and carcass quality. Despite extensive research efforts to evaluate the mechanisms by which stressors affect performance in many species, the long-standing problems within the livestock industries have not been alleviated. Stress, particularly early and sustained stress, results in immune dysfunction, Hypothalamic-Pituitary-Adrenalcortical (HPA) activity and an imbalance of chemicals in the brain. The nervous and immune systems are integrated and form an interdependent neuroimmune network. Depression, physical or emotional stresses activate the endocrine system altering immunological function, which in turn elicits physiological and chemical changes in the brain. Likewise, immunological stress in the form of infection activates the neuro-endocrine system via cytokines and other soluble mediators to induce stress responses which in turn impair productivity. Cytokines mediate interactions between the immune, endocrine and central nervous systems. Previously believed to be immuno-suppressive, there is mounting evidence that stress induces a shift in TH1/TH2 immune responses resulting in immune dysregulation rather than immunosuppression. The potential for cytokines to affect homeostatic pathways creates a need to evaluate the activities of the immune system.

### SUMMARY OF THE INVENTION

In its broadest aspect the present invention provides a method for improving the growth performance of an animal comprising the step of administering to an animal in need thereof a growth promoting amount of one or more anti-inflammatory agents.

The anti-inflammatory agents preferably increase or supplements the animals own anti-inflammatory systems.

The present invention also provides a method for improving the growth performance of an animal comprising the step of administering to an animal in need thereof a compound or composition which increases or supplements endogenous anti-inflammatory agent levels, wherein growth performance is enhanced relative to the growth performance of an animal which has not been administered said compound or composition.

Preferably, the compound or composition is administered prior to, together with, or subsequent to the administration of a growth promoting amount of one or more anti-inflammatory agents.

More preferably the compound or composition comprises antagonists of pro-inflammatory cytokine receptors. Even more preferably, the compound or composition comprises antagonists of TNF-α receptor, GM-CSF receptor, IL-6 receptor, IL-1 receptor, IL-4 receptor or IL-8 receptor. Most preferably the compound or -composition comprises IL-10, 1,8-napthosultam substituted compounds or quinoxaline compounds.

Alternatively, the compound or composition increases the endogenous level of anti-inflammatory agents by decreasing the amount of pro-inflammatory cytokines. Accordingly, the compound or composition comprises agents capable of increasing the amount of circulating, soluble cytokine receptors to pro-inflammatory cytokines.

The present invention also provides a method for improving the growth performance of an animal comprising the step of administering to an animal in need thereof a composition comprising an anti-inflammatory agent in conjunction with an antibiotic, optionally in combination with a pharmaceutical carrier, adjuvant or vehicle, wherein said composition achieves a synergistic growth promoting effect.

Preferably, the anti-inflammatory agent is any soluble cytokine receptor, cytokine receptor antagonist, cytokine inhibitory factor or biologically active fragment thereof which has an anti-inflammatory effect or an anti-inflammatory agent selected from the group consisting of diclofenac, diflunisal, etodolac, flunix, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic and tolmetin. Preferably, the soluble cytokine receptor or biologically active fragment thereof is selected from the group consisting of TNFα receptor, IL-6 receptor, IL-1 receptor, IL-4 receptor and IL-8 receptor or a combination thereof that are capable of improving the growth performance of an animal. More preferably, the soluble cytokine receptor or biologically active fragment thereof is IL-1 receptor.

Preferably, the cytokine receptor antagonist or biologically active fragment thereof is selected from the group consisting of IL-1ra, IL-6ra, IL-8ra and TNF-αra. More preferably, the cytokine receptor antagonist or biologically active fragment thereof is IL-1ra.

Preferably, the cytokine inhibitory factor or biologically active fragment thereof is selected from the group consisting of TNF blocking factor and TNF-alpha inhibitor.

In one particular embodiment, the anti-inflammatory agents of the present invention are formulated into a growth enhancing composition by combining one or more anti-inflammatory agents together with one or more pharmaceutical carriers, adjuvants or vehicles. More preferably, a growth enhancing composition is formulated by combining one or more soluble cytokine receptors, cytokine receptor antagonists, cytokine inhibitory factors or biologically active fragments thereof with either one or more other anti-inflammatory agents or pharmaceutical carriers, adjuvants or vehicles. Any known pharmaceutical carrier, adjuvant or vehicle may be used as long as it does not adversely affect the growth promoting effects of the anti-inflammatory agent(s).

Accordingly, in a second aspect the present invention provides a growth promoting composition comprising one or more anti-inflammatory agents together with one or more pharmaceutical carriers, adjuvants or vehicles. Preferably, the composition comprises anti-inflammatory agents selected from the group consisting of soluble cytokine receptor, cytokine receptor antagonist, cytokine inhibiting factor or biologically active fragment thereof, diclofenac, diflunisal, etodolac, flunix, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic and tolmetin.

More preferable, the composition comprises one or more soluble cytokine receptor, cytokine receptor antagonist, cytokine inhibitory factor or biologically active fragments thereof and one or more different soluble cytokine receptor, cytokine receptor antagonist, cytokine inhibitory factor or biologically active fragments thereof or one or more anti-inflammatory agent. Most preferably, the composition comprises one soluble cytokine receptor, cytokine receptor antagonist, cytokine inhibitory factor or biologically active fragment thereof and one different anti-inflammatory agent or a pharmaceutical carrier, adjuvant or vehicle.

Compositions comprising antibiotics assist in limiting the microbial load in an animal, thereby assisting the anti-inflammatory agent to improve growth performance in the animal. Particularly preferred antibiotics are those already in use in conventional animal production environments. However, in particular, the preferred antibiotic is selected from the group consisting of amoxycylin, ampicillin, apramycin, avoparcin, bacitracin, benethamine, benzathine, ceftiofur, cefuroxime, cephalonium, chlortetracycline, cloxacillin, dimetridazole, erythromycin, kitasamycin, lasalocid, lincomycin, monensin, narasin, neomycin, oleandomycin, oxytetracycline, olaquindox, penicillin, penicillin G, procaine, spectinomycin, streptomycin, tetracycline, tilmicosin, trimethoprim, tylosin, salinomycin, sulfonamides (including and diaveridine) and virginiamycin or combinations thereof. Most preferably, the antibiotic is amoxycylin, lincomycin or spectinomycin.

Depending upon the activity of the anti-inflammatory agent, manner of administration, age and body weight of the animal, different doses of anti-inflammatory agent can be used. Under certain circumstances, however, higher or lower doses may be appropriate. The administration of the dose can be carried out both by single administration in the form of an individual dose unit or else several smaller dose units and also by multiple administrations of subdivided doses at specific intervals.

It will be understood, however, that the specific dose level for any particular animal will depend upon a variety of factors including the activity of the specific anti-inflammatory agent employed, the age, body weight, general health, sex, diet, time of administration, and route of administration, rate of excretion and anti-inflammatory agent or antibiotic combination. However, generally the preferred route of administration is selected from the group consisting of oral, topical and parenteral administration.

Parenteral administration includes subcutaneous injections, aerosol, intravenous, intramuscular, intrathecal injection, infusion techniques or encapsulated cells.

The anti-inflammatory agents or compositions of the invention may also be administered as an additive to animal water and/or feed.

The growth performance of an animal may be determined by any know measure including increased growth rate, increased efficiency of feed use, increased final weight, increased dressed weight or decreased fat content. It will be further appreciated by those skilled in the art that the improved growth performance of an animal may result from immunoenhancement, anti-parasitic or anti-microbial effect, anti-inflammatory effect or stress reduction. More preferably, the immunoenhancement will result from a TH1/TH2 immune response, antibody isotype switching, hematopoiesis, improvement in immune function, mucosal immunity, beneficial affects on homeostatic processes such as appetite, endocrine or neural-endocrine processes.

It will be appreciated by those skilled in the art that the methods and compositions disclosed herein may be useful for any animal for which improving the growth performance is a desirable outcome. However, the present invention is particularly useful for feed animals ie those animals that are routinely farmed for meat production. Preferably, the animal is a higher artiodactyl or bird. Artiodactyls include cattle, pigs, sheep, camels, goats and horses. Birds include chickens, turkeys, geese, and ducks. More preferably, the present invention relates to animals selected from the group consisting of cattle, pigs, sheep, camels, goats, horses and chickens. Most preferably, the animals are cattle, pigs, or sheep.

In a third aspect, the anti-inflammatory agent is administered to an animal as a nucleic acid molecule encoding said anti-inflammatory agent such that upon expression of said nucleic acid molecule in the animal a growth promoting amount of the anti-inflammatory agent is produced. Thus, the present invention provides a method for improving the growth performance of an animal comprising the step of administering to an animal in need thereof a nucleic acid molecule encoding one or more anti-inflammatory agents, wherein the expression of said nucleic acid molecule produces an effective growth promoting amount of one or more anti-inflammatory agents.

The nucleic acid molecule may be DNA, cDNA, RNA, or a hybrid molecule thereof. It will be clearly understood that the term nucleic acid molecule encompasses a full-length molecule or a biologically active fragment thereof.

Preferably the nucleic acid molecule is a DNA molecule encoding a soluble cytokine receptor, cytokine receptor antagonist, cytokine inhibitory factor or biologically active fragment thereof. Most preferably, the DNA encodes a cytokine receptor selected from the group consisting of TNFα receptor, IL-6 receptor, IL-1 receptor, IL-4 receptor and IL-8 receptor or a.-combination thereof, or a cytokine receptor antagonist selected from the group consisting of IL-1ra, IL-6ra and TNF-αra.

The nucleic acid molecule may integrate into the animal genome, or may exist as an extrachromosomal element.

The nucleic acid molecule may be administered by any known method; however, it is preferably injected subcutaneously, intravenously, or intramuscularly or administered as an aerosol.

The amount of nucleic acid that is administered will depend upon the route and site of administration as well as the particular cytokine receptor, cytokine receptor antagonist, cytokine inhibitory factor or biologically active fragment thereof encoded by the nucleic acid molecule. As described herein, introducing an amount of 200 μg of a nucleic acid molecule encoding a cytokine receptor, cytokine receptor antagonist, cytokine inhibitory factor or biologically active fragment thereof is sufficient to improve growth performance in an animal. Thus, preferably the amount of about 200 μg to 1,000 μg of a nucleic acid molecule encoding a cytokine receptor, cytokine receptor antagonist, cytokine inhibitory factor or biologically active fragment thereof is preferably introduced into an animal.

The nucleic acid molecule may also be delivered in a vector such as a porcine adenovirus vector. It may also be delivered as naked DNA.

Accordingly, in fourth aspect, the present invention provides a construct for delivering in vivo an effective amount of a cytokine receptor, cytokine receptor antagonist, cytokine inhibitory factor or biologically active fragment thereof, comprising:

a) a nucleotide sequence encoding a cytokine receptor, cytokine receptor antagonist, cytokine inhibitory factor or biologically active fragment thereof;

b) a vector comprising a control sequence wherein the control sequence is capable of the controlling the expression of the nucleotide sequence of a) such that a cytokine receptor, cytokine receptor antagonist, cytokine inhibitory factor or biologically active fragment thereof is produced which in turns improves growth performance in an animal.

Modified and variant forms of the construct may be produced in vitro, by means of chemical or enzymatic treatment, or in vivo by means of recombinant DNA technology. Such constructs may differ from those disclosed, for example, by virtue of one or more nucleotide substitutions, deletions or insertions, but substantially retain a biological activity of the construct or nucleic acid molecule of this invention.

The present invention further provides kits. Accordingly, in a fifth aspect the invention provides a kit used for improving the growth performance of an animal comprising:

a). one or more anti-inflammatory agents;

b). a delivery device for said anti-inflammatory agents; and

c). instructions for use in the method of the invention.

Suitable buffering agents and ionic salts may also be included in the kit.

### BRIEF DESCRIPTION OF THE FIGURES

FIG. 1 shows the rate of gain over the first 4 weeks of the weaner phase for pigs treated with IL-1ra or saline, in the presence or absence of in-feed antibiotics. (Bars show group means and SEM).

FIG. 2 shows the rate of gain over weeks 5 and 6 of the weaner phase for pigs treated with IL-1ra or saline, in the presence or absence of in-feed antibiotics. (Bars show group means and SEM).

FIG. 3 shows the rate of gain over the weaner (D7-D42) and grower phases (D79 and D93) in pigs treated with IL-1ra or saline, in the presence or absence of in-feed antibiotics.

FIG. 4 shows the rate of gain over the finisher phase in pigs treated with IL-1ra or saline, in the presence or absence of in-feed antibiotics.

FIG. 5 shows the average weight at slaughter of pigs treated with saline or IL-1ra in the presence or absence of in-feed medication during the weaner phase.

FIG. 6 shows the average dressed weight (warm carcass weight) for pigs treated with saline or IL-1ra in the presence or absence of in-feed medication during the weaner phase.

FIG. 7 shows the feed conversion ratio for pigs treated with saline or IL-1ra in the presence or absence of in-feed medication during the weaner phase. Feed conversion was calculated over the finisher phase (day 93-day 133).

FIG. 8 shows mean weights at the end of the weaner phase in pigs treated with either IL-1ra or saline and provided with zero, reduced and normal levels of antibiotic medication.

FIG. 9 shows the production losses during the weaner phase in terms of incidence of weight loss and mortality in pigs treated with IL-1ra or saline and provided with zero, reduced or normal levels of antibiotic medication.

FIG. 10 shows the total group weight at the end of the weaner phase in pigs treated with IL-1ra or saline and provided with zero, reduced or normal levels of antibiotic medication.

FIG. 11 shows mean weights at the end of the grower phase in pigs treated with either IL-1ra or saline and provided with zero, reduced and normal levels of antibiotic medication.

FIG. 12 shows mean weights at the end of the finisher phase in pigs treated with either IL-1ra or saline and provided with zero, reduced and normal levels of antibiotic medication.

FIG. 13 shows mean P2 backfat measurements at slaughter in pigs treated with either IL-1ra or saline and provided with zero, reduced and normal levels of antibiotic medication.

FIG. 14 shows E. coli cultured from faeces collected from pigs treated with saline, IL-1ra or Apralan, for 5 days after initial challenge with E. coli. Data points show group means with standard errors.

FIG. 15 shows percentage reduction in total faecal culture scores over 5 days after E. coli challenge, compared to saline controls, in pigs treated with either IL-1ra or Apralan.

FIG. 16 shows recordings of diarrhoea and wet faeces for 5 days after E. coli challenge in pigs treated with IL-1ra, saline or Apralan. Bars show the total records for each group; the maximum records for any group is 40.

FIG. 17 shows percentage reduction in clinical signs (faecal condition) of E. coli infection in pigs treated with IL-1ra or Apralan, compared to saline controls.

FIG. 18 shows E. coli culture scores from samples taken in different areas along the gastro-intestinal tract at post-mortem in pigs treated with IL-1ra, saline or Apralan. SI refers to the small intestine. Bars show group means and standard errors.

FIG. 19 shows percentage reduction in E. coli culture scores at post mortem in pigs treated with either IL-1ra or Apralan, compared to saline treated controls.

FIG. 20 shows total E. coli culture scores from all areas of the gastro-intestinal tract at post-mortem in pigs treated with IL-1ra, saline or Apralan. Bars show group means and standard errors.

FIG. 21 shows percentage reduction in the total levels of E. coli present in the gut at post-mortem in pigs treated with IL-1ra or Apralan, compared to saline controls.

FIG. 22 shows E. coli culture scores at post-mortem from the foregut and hindgut in pigs treated with IL-1ra, saline or Apralan. Bars indicate groups mean and standard error.

FIG. 23 shows percentage reduction in E. coli culture scores obtained from the foregut and hindgut areas, in pigs treated with IL-1ra or Apralan, compared to saline controls.

FIG. 24 shows Spirochaete culture scores from samples taken in different areas along the gastro-intestinal tract at post-mortem in pigs treated with IL-1ra or saline. Bars indicate group mean.

FIG. 25 shows percentage reduction in spirochaete culture scores at post-mortem for pigs treated with IL-1ra compared to saline controls.

FIG. 26 shows faecal condition at post-mortem in pigs treated with saline or IL-1ra and challenged with swine dysentery.

FIG. 27 shows expression of mRNA for the pro-inflammatory cytokine TNFα in peripheral blood of pigs treated with IL-1ra or saline and challenged with swine dysentery.

FIG. 28 shows expression of mRNA for the pro-inflammatory cytokine IL-8 in peripheral blood of pigs treated with IL-1ra or saline and challenged with swine dysentery.

FIG. 29 shows expression of mRNA for the pro-inflammatory cytokine IL-1 in peripheral blood of pigs treated with IL-1ra or saline and challenged with swine dysentery.

FIG. 30 shows average weight gain for 2 weeks in pigs treated with recombinant IL-1ra, plasmid IL-1ra, the NSAID flunix, plasmid control or saline control and subsequently challenged with App. Bars indicate group mean and standard error.

FIG. 31 shows total weight gained during 14d challenge with App, in pigs treated with saline, flunix, recombinant IL-1ra, plasmid control or plasmid IL-1ra. Bars indicate group mean and standard error.

FIG. 32 shows daily rate of gain during 14d challenge with App, in pigs treated with saline, flunix, recombinant IL-1ra, plasmid control or plasmid IL-1ra. Bars indicate group mean and standard error.

FIG. 33 shows percentage change in weight gained compared to saline controls in pigs treated with either flunix or IL-1ra and subsequently challenged with App for 14d.

FIG. 34 shows percentage change in weight gained compared to saline controls and plasmid controls in pigs treated with IL-1ra plasmid and subsequently challenged with App for l4d.

FIG. 35 shows levels of TNFα protein in the serum of pigs treated with saline, flunix, recombinant IL-1ra, plasmid control of plasmid IL-1ra and subsequently challenged with App. Bars indicate group mean and standard error.

FIG. 36 shows expression of mRNA for the pro-inflammatory cytokine IL-6 in peripheral blood in pigs, treated with saline, flunix, recombinant IL-1ra, plasmid control or plasmid IL-1ra and challenged with App. NS refers to no sample for that time point. Bars indicate group mean and standard error.

FIG. 37 shows presence of clinical signs of App disease over 30 visits in the first week of challenge, in pigs treated with saline, flunix, recombinant IL-1ra, plasmid control or plasmid IL-1ra and challenged with App. Bars indicate group miean and standard error. The maximum possible score is 240.

FIG. 38 shows percentage reduction in clinical signs of disease in pigs treated with, flunix, recombinant IL-1ra, or plasmid IL-1ra and challenged with App, compared to the relevant control groups.

FIG. 39 shows degree of pleurisy at necropsy, expressed as pleurisy score (0-5) in pigs treated with saline, flunix, recombinant IL-1ra, plasmid control or plasmid IL-1ra and challenged with App. Bars indicate group mean and standard error.

FIG. 40 shows percentage reduction in pleurisy in pigs treated with flunix, recombinant IL-1ra or plasmid IL-1ra and challenged with App, compared to the relevant controls.

FIG. 41 shows degree of pleuropneumonia at necropsy, expressed as percentage of affected lung by weight, in pigs treated with saline, flunix, recombinant IL-1ra, plasmid control or plasmid IL-1ra and challenged with App. Bars indicate group mean and standard error.

FIG. 42 shows percentage reduction in affected lung mass in pigs treated with flunix, recombinant IL-1ra or plasmid IL-1ra and challenged with App, compared to the relevant controls.

FIG. 43 shows daily rate of gain in pigs treated with saline, low or high doses of IL-1ra, or IL-1ra+IL-4 (syn) during the first 10 days of App challenge. Bars indicate group mean and standard error.

FIG. 44 shows daily rate of gain in pigs treated with saline, low or high doses of IL-1ra, or IL-1ra+IL-4 (syn) during the second 10 days of App challenge. Bars indicate group mean and standard error.

FIG. 45 shows total weight gained in pigs treated with saline, low or high doses of IL-1ra, or IL-1ra+IL-4 (syn) during the total 21 days of App challenge. Bars indicate group mean and standard error.

FIG. 46 shows percentage improvement in weight gain compared to saline treated controls over 21 days of App challenge in pigs treated prophylactically with low or high doses of either IL-1ra, or IL-1ra+IL-4 (syn).

FIG. 47 shows amount of lung affected by App lesions, described as a percentage of total lung weight in pigs treated with saline, low or high doses of IL-1ra, or IL-1ra+IL-4 (syn) during the total 21 days of App challenge. Bars indicate group mean and standard error.

FIG. 48 shows pleurisy scores in lungs from pigs treated with saline, low or high doses of IL-1ra, or IL-1ra+IL-4 (syn) during the total 21 days of App challenge. Bars indicate group mean and standard error.

FIG. 49 shows expression of mRNA for the pro-inflammatory cytokine, IL-8, in lung tissue taken at postmortem from pigs treated with saline, low or high doses of IL-1ra, or IL-1ra+IL-4 (syn) during the total 21 days of App challenge. Bars indicate group mean and standard error.

FIG. 50 shows expression of mRNA for the pro-inflammatory cytokine, TNFα, in lung tissue taken at postmortem from pigs treated with saline, low or high doses of IL-1ra, or IL-1ra+IL-4 (syn) during the total 21 days of App challenge. Bars indicate group mean and standard error.

FIG. 51 shows weight gained in week 2 of App challenge in pigs subsequently treated with IL-1ra at high or low doses, saline or excenel. Bars indicate group means and standard error.

FIG. 52 shows feed intake in pigs challenged with App and subsequently treated with IL-1ra at high or low doses, saline or excenel. Bars indicate group means and standard error.

FIG. 53 shows feed conversion ratio pigs challenged with App and subsequently treated with IL-1ra at high or low doses, saline or excenel. Bars indicate group means and standard error.

FIG. 54 shows proliferative capacity of lymphocytes in response to stimulation with killed App, for pigs challenged with App and subsequently treated with IL-1ra at high or low doses, saline or excenel. Bars indicate group means and standard error.

FIG. 55 shows levels of mRNA for the pro-inflammatory cytokine IL-8, found in the lungs at post-mortem, in pigs challenged with App and subsequently treated with IL-1ra at high or low doses, saline or excenel. Bars indicate group means and standard error.

### DETAILED DESCRIPTION OF THE INVENTION

The practice of the present invention employs, unless otherwise indicated, conventional molecular biology, cellular biology, and recombinant DNA techniques within the skill of the art. -Such techniques are well known to the skilled worker, and are explained fully in the literature. See, e.g., Sambrook and Russell “Molecular Cloning: A Laboratory Manual” (2001) (Green Publishing, New York); Cloning: A Practical Approach,” Volumes I and II (D. N. Glover, ed., 1985) (Green Publishing, New York); “Oligonucleotide Synthesis” (M. J. Gait, ed., 1984); “Nucleic Acid Hybridisation” (B. D. Hames & S. J. Higgins, eds., 1985); “Antibodies: A Laboratory Manual” (Harlow & Lane, eds., 1988); “Transcription and Translation” (B. D. Hames & S. J. Higgins, eds., 1984); “Animal Cell Culture” (R. I. Preshney, ed., 1986); “Immobilised Cells and Enzymes” (IRL Press, 1986); B. Perbal, “A Practical Guide to Molecular Cloning” (1984), and Sambrook, et al., “Molecular Cloning: a Laboratory Manual” (1989). Ausubel, F. et al., 1989-1999, “Current Protocols in Molecular Biology” (Green Publishing, New York).

Before the present methods and compositions are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a soluble cytokine receptor” includes a plurality of such cytokine receptor, and a reference to “an antibiotic” is a reference to one or more antibiotics and equivalents thereof known to those skilled in the art, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

It is to be understood that the methods and compositions of the present invention are useful for improving the “growth performance” of an animal. The term “growth performance” is known in the art as a reference to the criteria of growth rate and efficiency of feed use of an animal, and also a reference to the final weight of an animal, and the dressed weight and fat content of a carcass from the animal. The “growth rate” of an animal is the rate of unit gain in live weight of the animal and “efficiency of feed use” is the amount of feed required per unit gain in live weight of the animal. The “final weight” of an animal is the weight of the animal at slaughter at a specified age and the “dressed weight” is the weight of a carcass from which viscera, feet, trotters or hooves have been removed. The “fat content” is the amount of fat on a dressed carcass. Methods for measuring the criteria of growth rate, efficiency of feed use, final weight, and dressed weight and fat content of a carcass, are known to the skilled worker. See, for example, Manipulating Pig Production VI, VII & VIII. 1997, 1999 & 2001, Ed. P. D. Cranwell, Australian Pig Science Association, Werribee, Victoria, Australia. Growth rate is obtained from successive measurements of live weight over time. Efficiency of feed use is obtained from successive measurements of feed disappearance and live weight over time. Carcass fat content is traditionally assumed from a measurement of back-fat thickness in millimetres at the P2 position. Accordingly, in the present invention the term “growth performance” means an improvement in one or more of the criteria of growth rate, efficiency of feed use, final or dressed weight and fat content of a carcass from an animal.

The term “animal” as used herein means any animal for which an increase in growth performance is desirable. For example, animals included in the mammalian order Artiodactyls or in the avian class Aves.

Artiodactyls comprise approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghorn (Antilocapridae), and cattle, sheep, goats and antelope (Bovidae). Many of these animals are used as feed animals in various countries. More importantly, with respect to the present invention, many of the economically important animals such as goats, sheep, cattle and pigs have very similar biology and share a high degree of genomic homology. More importantly, it is well known that certain animals such as goats and sheep and horses and donkeys can interbreed.

The terms “bird” and “avian” as used herein, are intended to include all avian species, including, but not limited to, chickens, turkeys, ducks, geese, quail, and pheasant which are commercially raised for eggs or meat. This term also includes both males and females of any avian species. Accordingly, the terms “bird” and “avian” are particularly intended to encompass hens, cocks and drakes of chickens, turkeys, ducks, geese, quail and pheasant. Chickens and turkeys are preferred.

All Artiodactyls have similar inflammatory systems which includes cytokine systems, in that they posses, for example, interleukins, GM-CSF, interferon's α, β and γ and their respective receptors. In most species the genes coding for these cytokines map to particular regions on certain chromosomes. For example, in humans, the interleukin 5 gene maps to chromosome 5q23-31 in the same area as genes encoding GM-CSF, M-CSF, IL-3 and IL-4. More importantly, many of the cytokines and their receptors have high degrees of amino acid sequence homologies between different species. For example, it is well known in the art that porcine interleukin 5 shares as much as 90% of its amino acids with animals such as bovine, ovine and equine (See, for example, Sylvin et al. (2000), Immunogenetics, 51: 59-64). Indeed, even species as distinct as mice and humans share as much as 70% amino acid sequence identities (See, for example, Dictionary of Cytokines (1995), Horst. Ibelgaufts, VCH Publishers, Weinheim). Furthermore, it is known that human IL-10 has a significant degree of sequence homology with bovine, murine, and ovine IL-10 (Dutia et al. (1994) Gene; 149:393-4).

It is also well known in the art that a number of cytokines have species cross-reactivity. For example, IL-4 has some cross-species reactivity, while IL-5 has a high level of cross-species reactivity Dictionary of Cytokines (1995), Horst Ibelgaufts, VCH Publishers, Weinheim. However, it should be noted that the cross-reactivity described in the prior art literature relates to in-vitro assays and some in-vivo experiments, but does not relate to growth performance.

Cytokines are also known to regulate the expression of cytokine receptors, either in a stipulatory or inhibitory manner, thereby controlling the biological activities of cytokines by other cytokines. Some cytokines share common receptor subunits which may have a regulatory effect. For example, the GM-CSF receptor shows significant homologies with other receptors for Hematopoietic growth factors, including IL-2-β, IL-3, IL-6, IL-7, Epo and the Prolactin receptors (See, for example, cytokines Online Pathfinder Encyclopaedia—www.copewithcytokines.de). It is also known that IL-3 is capable of upregulating the expression of GM-CSF receptors on mouse macrophages, IL-3 also upregulates IL-1 receptor expression on human and murine bone marrow cells, IL-4 upregulates IL-1 type 1 receptor expression and down regulate IL-2 receptor expression. Furthermore, IL-7 upregulates IL-4 Receptor expression, and TNFα upregulates both IL-3 and GM-CSF Receptor expression (Dictionary of Cytokines (1995), Horst Ibelgaufts, VCH Publishers, Weinheim).

In a similar fashion to Artiodactyls, birds also have common cytokine systems, including interleukins. Accordingly, the term “avian cytokine receptor,” or “bird cytokine receptor” as used herein, means any cytokine receptor corresponding to an cytokine produced by any avian species.

Given the level of common ancestry and biology for many of the feed animals, the high degree of amino acid sequence homology for cytokines and other inflammatory processes across a number of species such as cattle, sheep, goats and pigs, and the level of cross-species reactivity of the cytokines a person skilled in the art would appreciate that the compositions and methods disclosed herein are applicable for all feed animals and for all cytokine receptors.

It will be further appreciated by those skilled in the art that the compositions and methods disclosed herein may be directly extrapolated to encompass other aspects of the invention. For example, data are presented for specific cytokine receptor antagonists; however, these are not to be construed to be limiting on the invention. Indeed, the cytokine receptor antagonists disclosed were specifically chosen to illustrate the breadth of the invention. For example, many cytokines share receptors or receptor subunits. For example, IL-3, IL-5 and GM-CSF share a receptor subunit (Dictionary of -Cytokines (1995), Horst Ibelgaufts, VCH Publishers, Weinheim). IL-4 shares a common subunit with IL-2 and IL-7 (Dictionary of Cytokines (1995), Horst Ibelgaufts, VCH Publishers, Weinheim). Some cytokines have similar gene structures and are clustered on the one chromosome eg IL-3, IL-4, IL-5, GM-CSF and IL-13 in humans and mice (Dictionary of Cytokines (1995), Horst Ibelgaufts, VCH Publishers, Weinheim).

All of the foregoing is illustrative of the breadth of the presently disclosed invention with respect the types of animals encompassed. However, it will also be readily seen that the term “cytokine receptor” or “cytokine receptor antagonists” is also to be construed broadly and not limited to the experimental data disclosed. For example, the term “cytokine receptor” includes one or more of IL-1 receptor, IL-6 receptor, TGF-β receptor, IL-11 receptor, IL-18 receptor, IL-12 receptor, IL-17 receptor, LIF receptor, IPN-γ receptor IL-8 receptor, TNF-α receptor and GM-CSF receptor, in soluble form. The term “receptor antagonist” includes IL-1ra, IL-4ra, IL-8ra, GM-CSFra, IL-6ra or TNF-αra.

In one particularly preferred embodiment the initial step in the method of the invention involves the administration of a growth promoting amount one or more anti-inflammatory agents to an animal.

The term “anti-inflammatory agent” as used herein refers to any compound or composition which is capable of reducing inflammation. For example, soluble cytokine receptors, cytokine receptor antagonists, cytokine inhibitory factors or biologically active fragments thereof which have an anti-inflammatory effect may be used. Alternatively, an anti-inflammatory agent such as diclofenac, diflunisal, etodolac, flunix, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic or tolmetin may be used. Corticosteroid drugs are also known as powerful anti-inflammatory agents that are used widely to suppress the harmful effects of immune responses. Accordingly, in one embodiment corticosteroid drugs may used.

In a further embodiment, the term “anti-inflammatory agent” includes any compound or composition which increase the number of soluble receptors for pro-inflammatory cytokines.

As discussed above, growth performance is measurable; however, why there is an increase in growth performance is a little more complex. While not wishing to be bound by any particular theory or hypothesis, the applicant believes that the administration of an anti-inflammatory agent acts in a number of complementary ways that result in the improved growth performance. For example, the applicant has found that by improving the immunity of feed animals, stock losses are avoided and consequently growth performance improves. Thus, the present invention provides a method of reducing the susceptibility of an animal to infection. The method is useful for reducing susceptibility to infection by bacteria, virus or parasite.

The applicant has also found that the administration of one or more anti-inflammatory agents together with one or more antibiotics also improves the growth performance of an animal while reducing the total amount of antibiotic used. It is believed that antibiotic limits the microbial load in the animal to a threshold level at which the administered anti-inflammatory agents is then capable of exerting an effect on growth performance.

Accordingly, the applicant believes that rather than functioning as a growth promoter per se, although this may be possible, it will be understood that administration of the anti-inflammatory agents may cause improved growth performance by activating the humoral and cellular arms of the immune response which are capable of being activated by the anti-inflammatory agents.

As used herein, the term “Growth promoting amounts is meant an amount of an anti-inflammatory agent of the present invention effective to yield an increase in growth performance as defined above. For example, increased growth rate, efficiency of feed use, increased final weight, increased carcass dressed weight or reduced fat content.

As used herein, the term “administration” refers to the mode of delivery of a composition of the invention. The term also refers to the dosage of a composition. Depending upon the activity of an anti-inflammatory agent and age and body weight of an animal, the manner of administration and dosage of an anti-inflammatory agent will vary. It will be understood that the specific dose level for any particular animal will depend upon a variety of factors including the activity of the specific anti-inflammatory agent employed, the age, body weight, general health, sex, diet, time of administration, route of administration, rate of excretion and anti-inflammatory agent or antibiotic combination. However, generally the preferred route of administration is selected from the group consisting of oral, topical and parenteral administration.

Parenteral administration includes subcutaneous injections, aerosol, intravenous, intramuscular, intrathecal, injection or infusion techniques and encapsulated cells.

As used herein, the term “upregulate” or “upregulating” refers to inducing an increase in production, secretion or availability (and thus an increase in the concentration) of a protein or peptide. A method of upregulating endogenous anti-inflammatory agent in an animal or avian thus refers to a method of inducing an increase in the production, secretion or availability of anti-inflammatory agent in the animal or avian, as compared to an untreated animal or avian.

The term “endogenous” means originating within the subject, cell, or system being studied. Accordingly, supplementing the endogenous levels of an anti-inflammatory agent means that a compound or compounds is/are administered to an animal such that the total amount of an anti-inflammatory agent in the animal is higher than normally present. Increasing the endogenous levels of an anti-inflammatory agent means that a compound or compounds is/are administered to an animal where the compound or compounds increase the production of an anti-inflammatory agent by an animals cells or tissue, thereby effectively increasing the total amount of an anti-inflammatory agent in the animal. The endogenous levels of an anti-inflammatory agent may also be effectively increased by decreasing the turn over rate of a the anti-inflammatory agent. For example, a compound or compounds of the invention when administered to an animal may decrease the rate of proteolysis of endogenous anti-inflammatory agents by inhibiting the effect of proteolytic enzymes.

Many substances are able to stimulate upregulation of endogenous anti-inflammatory agents such as cytokine receptors, IL-4 and IL-16 or cytokine receptor antagonists. For example, as shown in International Patent Application No WO93/18783, IL-10 upregulates the expression on IL-1 receptor antagonist. Furthermore, compounds such 1,8-napthosultam substituted compounds or quinoxaline compounds are known to upregulate cytokine receptor antagonists such as IL-8. See, for example, International Patent Application Nos WO99/36070 and WO99/42461 herein incorporated by reference.

An alternative process of reducing or ameliorating the effects of pro-inflammatory cytokine such as IL-1, is by removing these from circulation. For example, it is well known that there are factors that are capable of binding to ligands thereby preventing them from binding their receptors. TNF blocking factor and TNF-α inhibitor, for example, discussed supra are known to bind to TNF.

The term “biologically active fragment” refers to a segment of an anti-inflammatory agent having a biological or physiological effect in an animal that is substantially similar to the entire or complete anti-inflammatory agent from which it is derived. For example, a biologically active fragment of IL-1 receptor antagonist may be any portion of IL-1 receptor antagonist having greater than about 5 amino acid residues which either comprises a biologically active site or wherein the portion retains IL-1 receptor antagonist activity. For example, if the IL-1 receptor antagonist portion retains the ability to bind to the IL-1 receptor as discussed above then this portion is a “biologically active fragment” of IL-1 receptor antagonist. Typically, such a fragment of IL-1 receptor antagonist is one capable of competitively inhibiting the binding of IL-1 to the IL-1 receptor.

It follows that a fragment of IL-1ra sufficient for providing some or all of IL-1ra function, or any other molecule sufficient for providing some or all of IL-1ra function, may be administered in the method, rather than IL-1ra. For example, such a fragment or molecule is capable of providing some or all of the function of IL-1ra including blocking IL-6 and IL-8 production. Typically, such a fragment or molecule is one capable of competitively inhibiting the binding of IL-1ra and/or IL-1 to the IL-1 receptor. Thus, in one embodiment, the invention comprises administering a fragment of IL-1ra sufficient for providing some or all of IL-1ra function, or a molecule sufficient for providing some or all of IL-1ra function.

Amino acid sequence variants of the amino acid sequence of a soluble cytokine receptor, cytokine receptor antagonist, cytokine inhibitory factor or biologically active fragment thereof are also encompassed. For example, where one or more amino acid residues are added at the N— or C-terminus of, or within, the soluble. cytokine receptor, cytokine receptor antagonist, cytokine inhibitory factor or biologically active fragment thereof sequence or its fragments as defined above. Amino acid sequence variants of a soluble cytokine receptor, cytokine receptor antagonist, cytokine inhibitory factor sequence or their fragments as defined above, wherein one or more amino acid residues of the sequence or fragment thereof are deleted, and optionally substituted by one or more amino acid residues; and derivatives of the above soluble cytokine receptor, cytokine receptor antagonist, cytokine inhibitory factor or biologically active fragment thereof, wherein an amino acid residue has been covalently modified so that the resulting product is a non-naturally occurring amino acid. Again all of these variants of soluble cytokine receptor, cytokine receptor antagonist, cytokine inhibitory factor or biologically active fragment thereof are encompassed by the term “biologically active fragment” as long as the variants retain the biological activity of the entire soluble cytokine receptor, cytokine receptor antagonist, cytokine inhibitory factor or biologically active fragment thereof from which it is derived.

As used herein, a “pharmaceutical carrier, adjuvant or vehicle” is a pharmaceutically acceptable solvent, suspending agent or vehicle for delivering the anti-inflammatory agent and/or antibiotic to the animal. The carrier may be liquid or solid and is selected with the planned manner of administration in mind.

The term “substantially homologous” can refer both to nucleic acid and/or amino acid sequences, means that a particular subject sequence, for example, a mutant sequence, varies from a reference sequence by one or more substitutions, deletions, or additions, the net effect of which does not result in an adverse functional dissimilarity-between reference and subject sequences. For purposes of the present invention, sequences having equivalent biological activity and equivalent expression characteristics are considered substantially homologous. Sequences having lesser degrees of identity, comparable bioactivity, and equivalent expression characteristics are considered equivalents.

“Microbial” refers to recombinant proteins made in bacterial, fungal (e.g., yeast), viral (e.g. baculovirus), or plant expression systems. As a product, “recombinant microbial” defines an animal protein essentially free of native endogenous substances and unaccompanied by associated native glycosylation. Protein expressed in most bacterial cultures, e.g., E. coli, will be free of glycosylation modifications; protein expressed in yeast and insect cells will have a glycosylation pattern different from that expressed in mammalian cells.

A “nucleic acid molecule” or “polynucleic acid molecule” refers herein to deoxyribonucleic acid and ribonucleic acid in all their forms, ie. single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus this term includes double-stranded DNA found, inter alia, in linear DNA molecules (eg. restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (ie. the strand having a sequence homologous to the mRNA).

A DNA sequence “corresponds” to an amino acid sequence if translation of the DNA sequence in accordance with the genetic code yields the amino acid sequence (ie. the DNA sequence “encodes” the amino acid sequence).

One DNA sequence “corresponds” to another DNA sequence if the two sequences encode the same amino acid sequence.

Two DNA sequences are “substantially similar” when at least about 85%, preferably at least about 90%, and most preferably at least about 95%, of the nucleotides match over the defined length of the DNA sequences. Sequences that are substantially similar can be identified in a Southern hybridization experiment, for example under stringent conditions as defined for that particular system. Defining appropriate hybridization-conditions is within the skill of the art. See eg. Sambrook et al., DNA Cloning, vols. I, II and III. Nucleic Acid Hybridization. However, ordinarily, “stringent conditions” for hybridization or annealing of nucleic acid molecules are those that

(1) employ low ionic strength and high temperature for washing, for example, 0.015M NaCl/0.0015M sodium citrate/0.1% sodium dodecyl sulfate (SDS) at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example is use of 50% formamide, 5×SSC (0.75M NaCl, 0.075M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2×SSC and 0.1% SDS.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (eg. a cDNA where the genomic coding sequence contains introns or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide in vivo. A polyadenylation signal and transcription termination sequence will usually be located 3′ to the coding sequence.

A “promoter sequence” is a DNA regulatory region capable of binding RNA polymerase in a cell and initiating transcription of a downstream (3′ direction) coding sequence. A coding sequence is “under the control” of the promoter sequence in a cell when RNA polymerase which binds the promoter sequence transcribes the coding sequence into mRNA, which is then in turn translated into the protein encoded by the coding sequence.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Bukaryotic promoters will often, but not always, contain “TATA” boxes and “CAT” boxes; prokaryotic promoters contain Shine-Delgarno sequences in addition to the −10 and −35 consensus sequences.

A cell has been “transformed” by exogenous DNA when such exogenous DNA has been introduced inside the cell wall. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eukaryotic cells, a stably transformed cell is one in which the exogenous DNA is inherited by daughter cells through chromosome replication. This stability is demonstrated by the ability of the eukaryotic cell to establish cell lines or clones comprised of a population of daughter cells containing the exogenous DNA.

“Integration” of the DNA may be effected using non-homologous recombination following mass transfer of DNA into the cells using microinjection, biolistics, electroporation or lipofection. Alternative methods such as homologous recombination, and or restriction enzyme mediated integration (REMI) or transposons are also encompassed, and may be considered to be improved integration methods.

A “clone” is a population of cells derived from a single cell or common ancestor by mitosis.

“Cell,” “host cell, ” “cell line,” and “cell culture” are used interchangeably herewith and all such terms should be understood to include progeny. A “cell line” is a clone of a primary cell that is capable of stable growth in vitro for many generations. Thus the words “transformants” and “transformed cells” include the primary subject cell and cultures derived therefrom, without regard for the number of times the cultures have been passaged. It should also be understood that all progeny might not be precisely identical in DNA content, due to deliberate or inadvertent mutations.

Vectors are used to introduce a foreign substance, such as DNA, RNA or protein, into an organism. Typical vectors include recombinant viruses (for DNA) and liposomes (for protein). A “DNA cloning vector” is an autonomously replicating DNA molecule, such as plasmid, phage or cosmid. Typically the DNA cloning vector comprises one or a small number of restriction endonuclease recognition sites, at which such DNA sequences may be cut in a determinable fashion without loss of an essential biological function of the vector, and into which a DNA fragment may be spliced in order to bring about its replication and cloning. The cloning vector may also comprise a marker suitable for use in the identification of cells transformed with the cloning vector.

An “expression vector” is similar to a DNA cloning vector, but contains regulatory sequences which are able to direct protein synthesis by an appropriate host cell. This usually means a promoter to bind RNA polymerase and initiate transcription of mRNA, as well as ribosome binding sites and initiation signals to direct translation of the mRNA into a polypeptide. Incorporation of a DNA sequence into an expression vector at the proper site and in correct reading frame, followed by transformation of an appropriate host cell by the vector, enables the production of mRNA corresponding to the DNA sequence, and usually of a protein encoded by the DNA sequence.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease Si), as well as protein binding domains (consensus sequences) responsible for the binding of RNA polymerase.

An “exogenous” element is one that is foreign to the host cell, or is homologous to the host cell but in a position within the host cell in which the element is ordinarily not found.

“Digestion” of DNA refers to the-catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearized DNA fragments (restriction fragments). The various restriction enzymes used herein are commercially available, and their reaction conditions, cofactors, and other requirements as established by the enzyme manufacturers are used. Restriction enzymes are commonly designated by abbreviations composed of a capital letter followed by other letters representing the microorganism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“Oligonucleotides” are short-length, single- or double-stranded polydeoxynucleotides that are chemically synthesized by known methods (involving, for example, triester, phosphoramidite, or phosphonate chemistry), such as described by Engels, et al., Agnew. Chem. Int. Ed. Engl. 28:716-734 (1989). They are then purified, for example, by polyacrylamide gel electrophoresis.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. Wang, et al., in PCR Protocols, pp. 70-75 (Academic Press, 1990); Ochman, et al., in PCR Protocols, pp. 219-227; Triglia, et al., Nucl. Acids Res. 16:8186 (1988).

“PCR cloning” refers to the use of the PCR method to amplify a specific desired nucleotide sequence that is present amongst the nucleic acids from a suitable cell or tissue source, including total genomic DNA and cDNA transcribed from total cellular RNA. Prohman, et al., Proc. Nat. Acad. Sci. USA 85:8998-9002 (1988); Saiki, et al., Science 239:487-492 (1988); Mullis, et al., Meth. Enzymol. 155:335-350 (1987).

A “vector” or “construct” refers to a plasmid or virus or genomic integration comprising a transcriptional unit with (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems would include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product. Generally, recombinant expression vectors will include origins of replication and selectable markers permitting transformation of the host cell, and a promoter derived from a highly-expressed gene to induce transcription of a downstream structural sequence. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product. Preferred recombinant expression vectors of the invention are viral vectors (eg. porcine adenoviral vector, mammalian cells (eg. porcine cells), plant cells and bacterial cells.

The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte proliferation), as defined herein below.

The term “systemic immune response” is meant to refer to an immune response in the lymph node-, spleen-, or gut-associated lymphoid tissues wherein cells, such as B lymphocytes, of the immune system are developed. For example, a systemic immune response can comprise the production of serum IgG's. Further, systemic immune response refers to antigen-specific antibodies circulating in the blood stream and antigen-specific cells in lymphoid tissue in systemic compartments such as the spleen and lymph nodes. In contrast, the gut-associated lymphoid tissue (GALT) is a component of the mucosal immune system since antigen-specific cells that respond to gut antigens/pathogens are induced and detectable in the GALT.

As cytokine receptor and cytokine receptor antagonist are endogenously expressed in all feed animal species and that many of these have a high degree of cross-reactivity, it follows that cytokine receptors and cytokine receptor antagonists from one species may be administered to animals of a different species and vice versa. For example, when the animal is a pig, human cytokine receptors such as IL-1 receptor may be used in the disclosed methods. There is no requirement that the particular cytokine receptor or cytokine receptor antagonist is identical to the cytokine receptor or cytokine receptor antagonist which is endogenously expressed in the animal.

The methods of this invention involve in one embodiment:

(1) The administration of one or more anti-inflammatory agents, prior to, together with, or subsequent to the administration of one or more antibiotics; or

(2) The administration of a composition comprising 30 one or more anti-inflammatory agents and one or more antibiotics.

(3) The administration of one or more anti-inflammatory agents without any antibiotics.

The anti-inflammatory agent(s) or composition(s) 35 of the invention may be administered orally, topically, or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, injection or infusion techniques.

The present invention also provides suitable topical, oral, and parenteral pharmaceutical formulations for use in the novel methods of improving growth performance of the present invention. The compositions of the present invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable carriers, adjuvants or vehicles which are suitable for the manufacture of tablets.

These carriers, adjuvants or vehicles may be, for example, (1) inert diluents, such as calcium carbonate, lactose, calcium phosphate or sodium phosphate; (2) granulating and disintegrating agents, such as corn starch or alginic acid; (3) binding agents, such as starch, gelatine or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The anti-inflammatory agents as well as the antibiotics useful in the methods of the invention can be administered, for in vivo application, parenterally by injection or by gradual perfusion over time independently or together. Administration may be intravenously, intra-arterial, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The invention includes various compositions useful for improving growth performance. The compositions according to one embodiment of the invention are prepared by bringing one or more anti-inflammatory agents, with or without one or more antibiotics into a form suitable for administration to an animal using carriers, adjuvants, vehicles or additives.

Antibiotics suitable for use in this aspect of the invention are those conventionally used in animal husbandry as an additive to animal water and/or feed and for limiting microbial load in the animal. Examples of these antibiotics include lincomycin, spectinomycin and amoxycillin. A detailed analysis of antibiotic usage for food-producing animals in Australia is described in “The use of antibiotics in food-producing animals: antibiotic-resistant bacteria in animals and humans”. Report of the Joint Expert Advisory Committee on Antibiotic Resistance (JETACAR), Commonwealth of Australia, 1999.

An antibiotic can be administered to the animal in an amount that is the same as the amount which would be conventionally administered to the animal for the purpose of decreasing microbial load in the animal. These amounts of antibiotic are known to the skilled worker and referred to in JETACAR above.

Frequently used carriers, adjuvants or vehicles include magnesium carbonate, titanium dioxide, lactose, mannitol and other sugars, talc, milk protein, gelatine, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions according to the invention may be administered locally or systemically in a growth promoting amount. Amounts effective for this use will, of course, depend on the anti-inflammatory agent and the weight and general state of the animal. Typically, dosages used in vitro may provide useful guidance in the amounts useful for in situ administration of the compositions. Various considerations are described, eg., in Langer, Science, 249: 1527, (1990). Formulations for oral use may be in the form of hard gelatine capsules wherein the active ingredient is mixed with an inert solid diluent, for example, calcium carbonate, calcium phosphate or kaolin. They may also be in the form of soft gelatine capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The compositions may be in the form of a sterile injectable aqueous or oleagenous suspension. This suspension may be formulated according to known methods using those suitable dispersing or wetting agents and suspending agents which have been mentioned above. The sterile injectable preparation may also a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Anti-inflammatory agents and compositions of the invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the anti-inflammatory agents or compositions of the present invention are of the order of about 1 microgram to about 50 microgram per kilogram body weight, with a preferred dosage range between about 5 microgram to about 20 microgram per kilogram body weight per—dose (could be multiple or single) (from about 100 micrograms to about 500 micrograms per animal per dose). The amount of anti-inflammatory agent that may be combined with the carrier materials to produce a single dosage will vary depending upon the animal and the particular mode of administration. For example, a formulation intended for intravenous administration to a pig may contain about 20 μg to 1 g of anti-inflammatory agent with an appropriate and convenient amount of carrier material which may vary from about 5 to 95 percent of the total -composition. Dosage unit forms will generally contain between from about 5 μg to 500 mg of anti-inflammatory agent.

It will be understood, however, that the specific dose level for any particular animal will depend upon a variety of factors including the activity of the specific anti-inflammatory agent employed, the age, body weight, general health, diet, time of administration, route of administration, rate of excretion and drug combination.

In one particularly preferred embodiment of the present invention the anti-inflammatory agent or agents are expressed in vivo rather than administered exogenously. For example, by inserting a structural DNA sequence encoding an anti-inflammatory agent together with suitable translation initiation and termination signals in operable reading phase with a functional promoter an expression vector is created which would be able to express the anti-inflammatory agent in vivo. The vector will comprise one or more phenotypic selectable markers and an origin of replication to ensure amplification within the host. Suitable prokaryotic hosts for transformation include E. coli, Bacillus subtilis, Salmonella typhimurium and various species within the genera Pseudomonas, Streptomonas; and Staphylococcus, although others may also be employed as a matter of choice. Following transformation of a suitable host strain and expression, the cells are cultured for an additional period. Cells are typically harvested by centrifugation, disrupted by physical or chemical means, and the resulting crude extract retained for further purification. Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines and of course porcine cells. Mammalian expression vectors will comprise an origin of replication, a suitable promoter, and enhancer, and also any necessary ribosome binding sites, polyadenylation sites, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements. Recombinant protein produced in bacterial culture is usually isolated by initial extraction from cell pellets, followed by one or more salting out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents. Use of an expression system that expresses a tag sequence for purification would simplify purification. Recombinant expression systems as defined herein will express heterologous protein upon induction of the regulatory elements linked to the DNA segment or synthetic gene to be expressed. Cell-free translation systems can also be employed to produce porcine cytokines using RNAs derived from the DNA constructs of the present invention. Appropriate cloning and expression vectors for use with prokaryotic and eukaryotic hosts are described by Maniatis, Molecular Cloning: A Laboratory Manual, (Cold Spring Harbor, N.Y., 1985), the disclosure of which is hereby incorporated by reference.

The nucleic acid encoding a particular anti-inflammatory agent is advantageously in the form of plasmid DNA or a viral vector (which vector is derived from an adenovirus, retrovirus, poxvirus, in particular from a vaccinia virus or an MVA virus, herpes virus, adenovirus-associated virus, etc.). The nucleic acid encoding a particular anti-inflammatory agent is transported by means of an infectious viral particle or in the form of a synthetic vector (cationic lipid, liposome, cationic polymer, etc.) or an engineered cell (cell which is transfected or transduced with the said nucleic acid) or non-engineered cell (which naturally contains the said nucleic acid).

According to an additionally preferred variant, the nucleic acid of interest is carried by an adenoviral vector which is defective for replication (unable to replicate autonomously in a host cell). The technology of adenoviruses is described in the state of the art (see, for example, Graham and Prevec in Methods in Molecular Biology, 1991, vol 7, pp. 109-128, ed E. J. Murey, The Human Press Inc). Advantageously, the adenoviral vector which is used within the context of the present invention is derived from the genome of an adenovirus, comprises at least the ITRs (inverted terminal repeats) and an encapsidation sequence and lacks all or part of the E1 adenoviral region. In addition, it can lack all or part of the E3 adenoviral region. However, according to an advantageous embodiment, preference is given to retaining the part of the E3 region which encodes polypeptides, in particular the glycoprotein gp19 k (Gooding et al., Critical Review of Immunology, 1990, 10: 53-71), which make it possible to escape the immune system of the host. Furthermore, the vector can contain additional deletions or mutations which affect, in particular, all or part of one or more regions selected from the E2, E4, L1, L2, L3, L4 and L5 regions (see, for example, international application WO 94/28152). In order to illustrate this point, mention may be made of the temperature-sensitive mutation which affects the DBP (standing for DNA-binding protein) gene of the E2 A region (Ensinger et al., J. Virol., 1972, 10: 328-339). Another variant, or attractive combination, consists in deleting the E4 region with the exception of the sequences which encode open reading frames (ORFs) 6 and 7 (these limited deletions do not require the E4 function to be complemented;. Ketner et al., Nucleic Acids Res., 1989, 17: 3037-3048). Preferably, the gene(s) of interest is/are inserted into the vector in place of the deleted adenoviral regions, in particular the E1 region. When several genes of interest are used, they can be inserted at the same site or at different sites in the viral genome and can be under the control of the same regulatory elements or of independent elements and, where appropriate, some of them can be in the opposite orientation to the others in order to minimize the phenomena of interference at the level of their expression. The genome of the recombinant adenoviral vector can be prepared by molecular biology techniques or by homologous recombination (see WO 96/17070).

The adenoviral vectors which are used within the context of the present invention are propagated in a complementing cell line which is able to supply the defective function(s) in trans in order to produce the peptides which are required for forming the infectious viral particles. For example, use will be made of cell line 293 for complementing the E1 function (Graham et al., J. Gen. Virol., 1977, 36: 59-72) or of the cell lines described in international application WO 97/04119 for effecting a double complementation. It is also possible to employ an appropriate cell line and a helper virus in order to complement all the defective functions. The viral particles which are produced are recovered from the cell culture and, if need be, purified using the techniques of the art (caesium chloride gradient, chromatographic steps, etc.).

The adenoviral vector which is used within the context of the present invention can be derived from the genome of an adenovirus of human, canine, avian, bovine, murine, ovine, porcine or simian origin or else from a hybrid which comprises adenoviral genome fragments of different origins. Mention may be made, more specifically, of the CAV-1 or CAV-2 adenoviruses of canine origin, of DAV of avian origin, or else of type 3 Bad of bovine origin (Zakharchuk et al., Arch. Virol., 1993, 128: 171-176; Spibey and Cavanagh, J. Gen. Virol., 1989,. 70: 165-172; Jouvenne et al., Gene, 1987, 60: 21-28; Mittal et al., J. Gen. Virol., 1995, 76: 93-102). However, preference will be given to an adenoviral vector that is specific for the particular animal species being studies. For example, porcine adenovirus (PAV) would be administered to pigs.

The method and means of the present invention may be embodied in the form of a kit.

The kit comprises a first container containing one or more anti-inflammatory agents, a device for delivering the agents and instructions for use.

In embodiments adapted -for use in intensive animal production, the kit might additionally comprise a second container containing one or more antibiotics. An alternate kit would comprise a first container containing one or more nucleic acid molecules encoding anti-inflammatory agents, which when administered to an animal would, upon expression of said nucleic acid molecule in the animal, produce a growth promoting amount of the anti-inflammatory agent, a device for delivering the nucleic acid molecules and instructions for use.

The instructions for use would enable a farmer or other animal husbandry practitioner to administer the anti-inflammatory agent or nucleic acid molecules such that growth promotion of the animal is enhanced relative to an animal that is not administered such agents or nucleic acid molecules.

Throughout the specification, the word “comprise” and variations of the word, such as “comprising” and “comprises”, means “including but not limited to” and is not intended to exclude other additives, components, integers or steps.

The invention will now be further described by way of reference only to the following non-limiting examples. It should be understood, however, that the examples following are illustrative only, and should not be taken in any way as a restriction on the generality of the invention described above. For example, while the majority of the examples relate to pigs, it is to be understood that the invention can also be applied to other animals as disclosed herein, including for example, sheep, cattle and chickens.

### EXAMPLE 1

### Recombinant IL-1RA as a Growth Promotant to Replace In-Feed Antibiotics

This experiment was performed to determine whether IL-1ra is able to improve the growth performance and health of weaner pigs in a commercial environment. We also wished to investigate the potential of IL-1ra as a replacement for antibiotic medication in feed.

Experiment Design

Recombinant porcine. IL-1ra was expressed in E. coli and purified using a polyhis tag system. IL-1ra was tested for biological activity in a bioassay prior to the start of the experiment.

Male weaner pigs (28 days old) were allocated to treatment groups of 20 as described in Table 1. The mean weight for each treatment group was equal, with equal variance. Pigs were housed in group pens of 20, with 2 pens provided with medicated water and feed as per current industry standards, while 2 pens were given unmedicated water and feed. Pigs were injected twice weekly with recombinant IL-1ra or saline (control), in a volume of 1 ml, for the duration of the weaner phase (42 days), as described in Table 2.

Upon commencement of the weaner phase (day 0) and upon completion of the weaner phase (day 42), pigs were

TABLE 1

| Group | treatment | medication |
|---|---|---|
| Saline+ | 1 ml saline | Yes |
| IL-1ra+ | 200 μg IL-1ra | Yes |
| | in 1 ml | |
| | saline | |
| Saline− | 1 ml saline | No |
| IL-1ra− | 200 μg IL-1ra | No |
| | in 1 ml | |
| | saline | |

TABLE 2

| | |
|---|---|
| Day 0 | Weighed and grouped 28 day old weaners. |
| | Bleed. Injected Groups. |
| Day 1 | Injected Groups. |
| Day 6 | Weighed |
| | Injected Groups. |
| Day 7 (Week 1) | Injected Groups. |
| Day 9 | Weighed |
| Day 13 | Injected Groups. |
| | Injected Groups. |
| Day 14 (Week 2) | Weighed |
| Day 16 | Injected Groups. |
| Day 20 | Injected Groups. |
| | Weighed |
| Day 21 (Week 3) | Injected Groups. |
| Day 23 | Injected Groups. |
| Day 27 | Weighed |
| | Injected Groups. |
| Day 28 (Week 4) | Injected Groups. Weighed. Final bleed. |
| Day 30 | Moved to grower pens. |
| Day 34 | Grower stage. All pigs given standard |
| | feed and remained in previous groups. |
| Day 35 (Week 5) | Weighed during (D73) and end of grower |
| Day 37 | stage (D93). |
| Day 41 | Finisher stage. Pigs moved into single |
| | pens and feed intake measured for FCR |
| | (food conversion ratio). All pigs given |
| Day 42 (Week 6) | standard finisher feed. Weighed during |
| (Days 42-93) | (day of experiment (D) 114) and end of |
| | finisher stage (Slaughter D133). |
| | Measured final weight, P2 backfat, |
| (Days 93-133) | carcass weight, % dressing. |

bled by venipuncture for immunological analyses. Pigs were weighed weekly throughout the weaner phase (day 0-day 42), and then on day 79, 93 and at termination of the trial on day 113, after which, animals were slaughtered and carcass characteristics measured.

Results

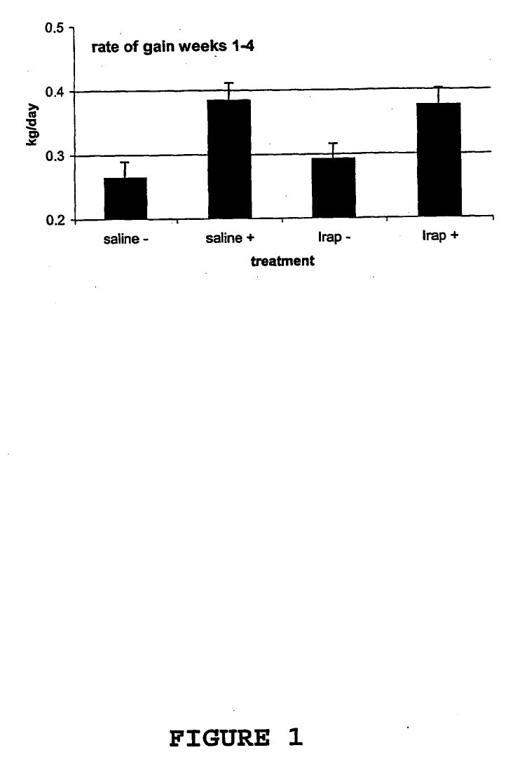

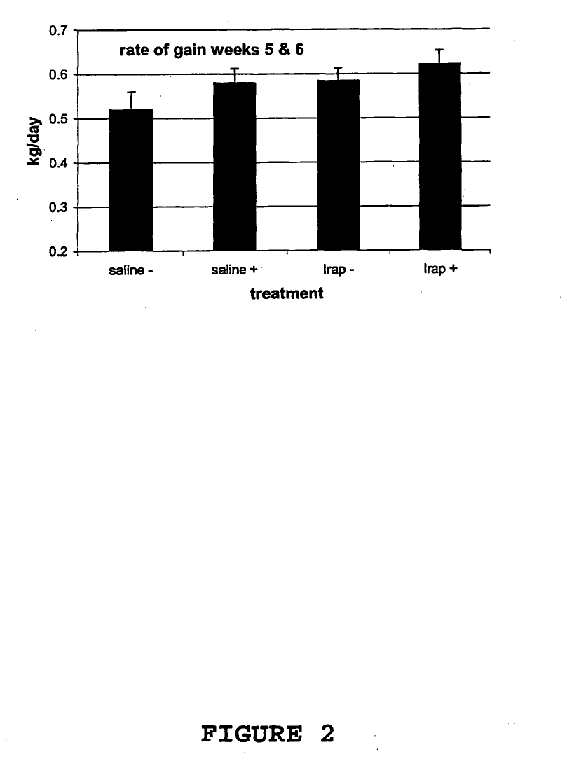

For the first 4 weeks of treatment, pigs treated with IL-1ra performed as well as saline controls for rate of gain (FIG. 1). However, as the experiment and treatments progressed into the later weeks of the weaner stage, weeks 5 and 6, pigs treated with IL-1ra were outperforming saline treated pigs in both medicated and non-medicated feeding regimes (FIG. 2). Furthermore, pigs treated with IL-1ra and fed a diet free of antibiotics performed as well as saline treated pigs fed a medicated diet (FIG. 2, IL-1ra—vs saline+). Pigs treated with IL-1ra and fed a medicated diet had the greatest rate of gain over the final 2 weeks of weaning.

Such results suggest that IL-1ra treatment is as effective as in-feed antibiotic application for promoting the growth of pigs under commercial conditions. The improved performance of IL-1ra treated pigs after 4 weeks of IL-1ra administration also implies that the effect of IL-1ra is delayed, or requires several treatments to produce the similar effects as in-feed antibiotics

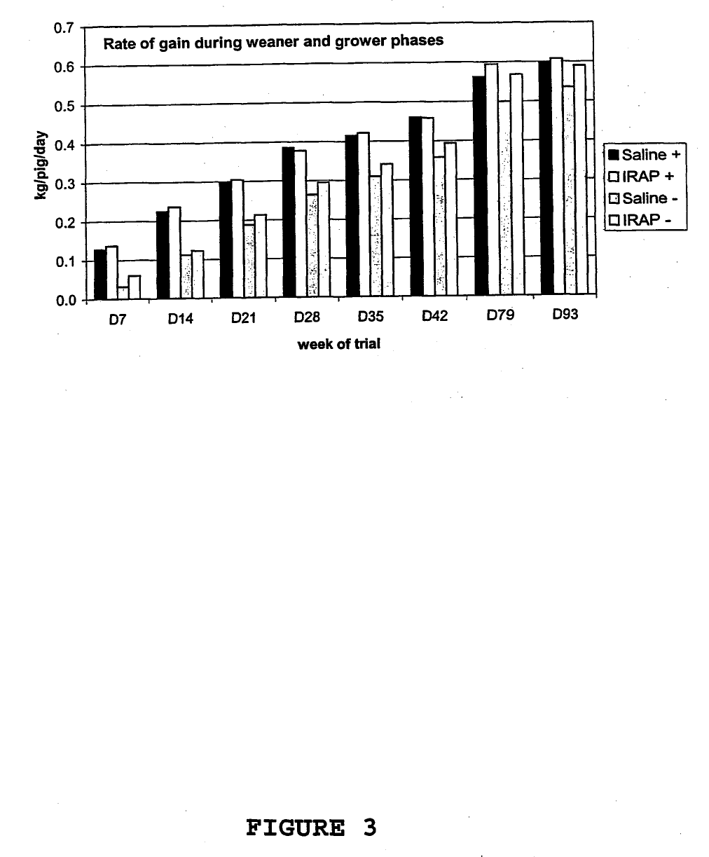

The trend for higher rate of gain in IL-1ra treated pigs continued through the grower and finisher phases (days 43-92, and days 93-134 respectively). After injections ceased in week 6, pigs treated with IL-1ra without in-feed medication proceeded to grow as quickly as pigs treated with saline and provided with in-feed medication (FIG. 3). Again, this result implies that treatment with IL-1ra is as efficacious as antibiotics for promoting increased growth in pigs, as shown in the last 2 weeks of the weaner phase (FIG. 2) and in the grower phase (FIG. 3, D79 and D93). The effect of IL-1ra appears to be long-lived after the cessation of cytokine treatment in week 6.

Pigs treated with IL-1ra and provided with a medicated diet showed the highest rate of gain over the grower phase (FIG. 3, D79 and D93), while pigs treated with saline and fed an unmedicated diet had the lowest rate of gain of all treatment groups.

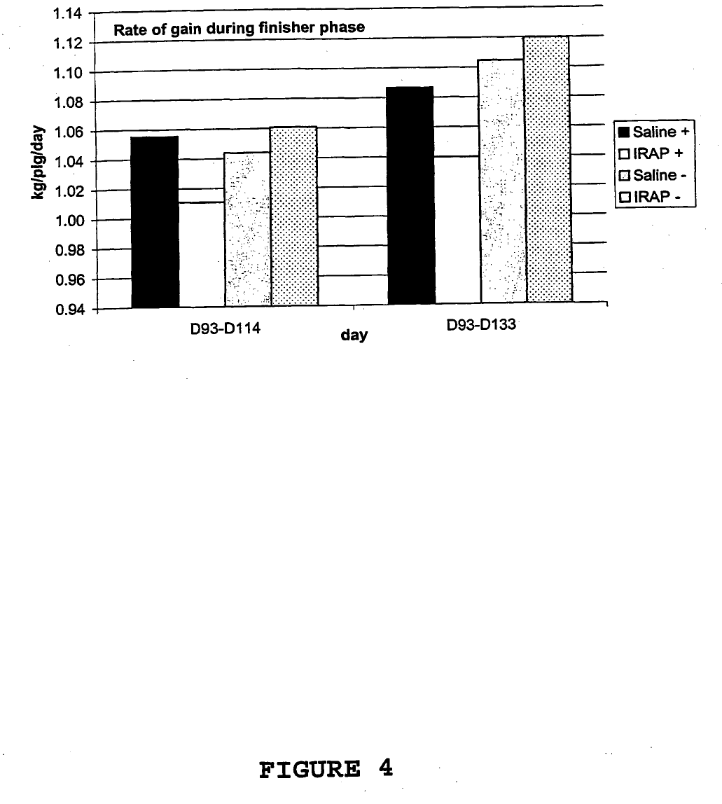

In the finisher phase, pigs treated with IL-1ra during the weaner phase without a medicated diet showed the highest rate of gain of all groups (FIG. 4). This result further illustrates the delayed nature of response to IL-1ra, which was administered until week 6, and that IL-1ra is as effective a promoter of growth in pigs as the in-feed antibiotics currently used by industry.

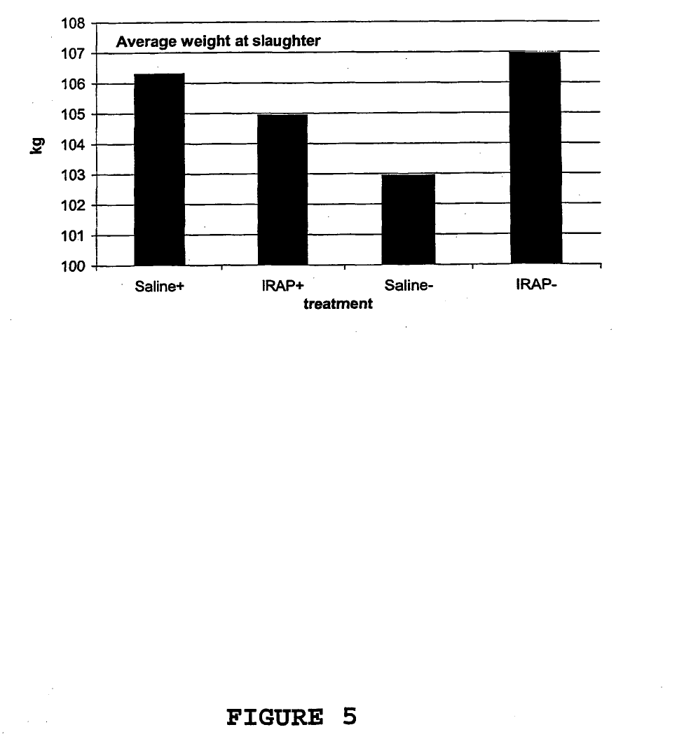

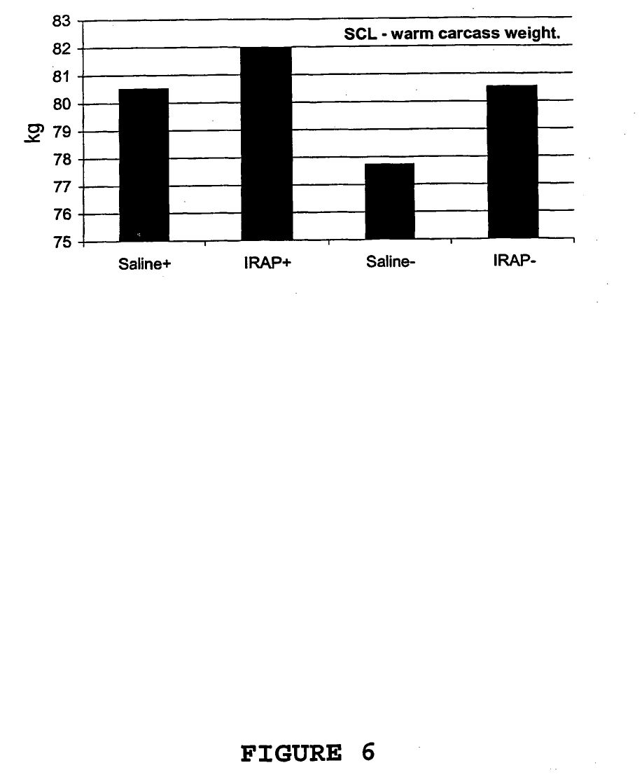

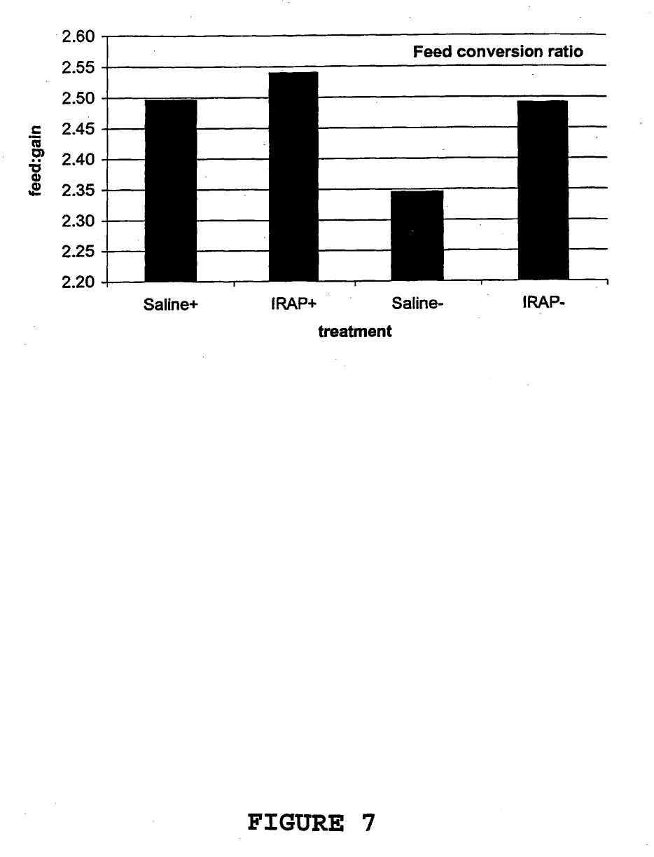

The positive effects of IL-1ra as a growth promotant are also indicated by the average weight of pigs at slaughter (FIG. 5), and their subsequent warm carcass weight after slaughter (FIG. 6). Pigs treated with IL-1ra in the weaner phase in the absence of in-feed antibiotics were on average 0.7 kg heavier at slaughter than were pigs treated with saline and provided with in-feed antibiotics. This increase in weight with IL-1ra treatment occurred without any difference in feed conversion ratio between these two treatments (FIG. 7). Pigs provided with in-feed antibiotics during the weaner phase had a feed conversion ratio of 2.5 during the finisher phase, while pigs treated with IL-1ra in the absence of in-feed antibiotics had a feed conversion ratio of 2.49 over the finisher phase. The warm carcass weight of pigs treated with IL-1ra in the presence of antibiotics was greater than that of their saline-treated counterparts (FIG. 6). Further, the warm carcass weight of pigs treated with IL-1ra in the absence of antibiotics was equal to that obtained by pigs fed antibiotics and treated with saline. These results illustrates that while IL-1ra treated pigs gained more weight than antibiotic treated pigs, this gain was as efficient as the current commercial practice of feeding pigs antibiotics.

Conclusions

1). IL-1ra improved growth in pigs in the absence of in-feed antibiotics.

2). the improvement in growth produced by IL-1ra treatment was equal to that seen by the addition of in-feed antibiotics.

3). IL-1ra improved growth in the last 2 weeks of the weaner phase in the absence of in-feed antibiotics, compared to saline treated controls, and saline treated pigs fed an antibiotic supplemented diet.

4). The effect of IL-1ra administration during the weaner phase on growth was delayed and of long duration, continuing throughout the grower and finisher phases.

5). IL-1ra treated pigs grew as well as antibiotic fed pigs in the grower phase.

6). IL-1ra treated pigs grew faster than antibiotic fed pigs in the finisher phase.

7). IL-1ra administration in the weaner phase resulted in increased slaughter weights compared to pigs treated with saline and fed a diet supplemented with antibiotics.

8). Pigs treated with IL-1ra in the weaner phase had the same feed efficiency during the finisher phase as pigs fed an antibiotic supplemented diet during the weaner phase.

9). These results indicate that IL-1ra produces larger pigs without affecting feed conversion efficiency, than does the current industry practice of supplementing pig diets with antibiotics.

### EXAMPLE 2

### Recombinant IL-1RA as a Growth Promotant to Reduce Levels of In-Feed Antibiotics

This experiment repeats the evaluation of IL-1ra to improve the performance and/or immunity of pigs by comparing the growth rate and health of male and female weaner pigs through the weaner, grower and finisher phases through to slaughter. This trial was designed to investigate the effect of providing IL-1ra at several levels of medication, from normal levels of antibiotic medication currently used in pig production through to reduced antibiotic medication and absence of antibiotic medication. This experiment evaluates the capacity of IL-1ra to replace antibiotics under normal commercial pig rearing conditions, and determined the effect of continuous administration of IL-1ra throughout the life of the animal.

Experiment Design

Recombinant porcine IL-1ra was expressed in E. coli and purified using a polyhis tag system. IL-1ra was tested for biological activity in a bioassay prior to the start of the experiment.

The experiment was undertaken in a commercial environment where the pigs were weaned at 28 days of age. All injections were 1 ml. There were 16 pigs per treatment, 8 males and 8 females per treatment. The mean weight of treatment groups were similar at the start of the experiment.

Treatment Protocol

| Group | Treatment | Medication |
|---|---|---|
| 1 | saline | 0 |
| 2 | saline | reduced |
| 3 | saline | normal |
| 4 | IL-1ra | 0 |
| 5 | IL-1ra | reduced |
| 6 | IL-1ra | normal |

Symbols Used

indicates no antibiotic supplements in feed or water throughout trial.

0.5 indicates single antibiotics used throughout trial.

+ indicates normal commercial antibiotic regime used throughout trial.

Treatments

A. Saline injection, 1 ml IM neck muscle.

B. 100 ug IL-1ra injection, 1 ml IM neck muscle. Injections were administered twice weekly during the weaner stage, and weekly throughout the grower and finisher stages.

Pigs were weaned and weighed at the commencement of the experiment (D0, W0). Weights were recorded weekly throughout the weaner phase (W0-W6), during the grower phase (W9), at the end of the grower phase (W13), during the finisher phase (W 16) and at the end of the finisher phase prior to slaughter at W19. Blood samples were collected at the start of the experiment and at the end of the weaner phase, grower phase and finisher phase. Haematology and immunological analyses were performed. At slaughter, carcass characteristics including P2 backfat measurements and dressed carcass weight were noted.

Results

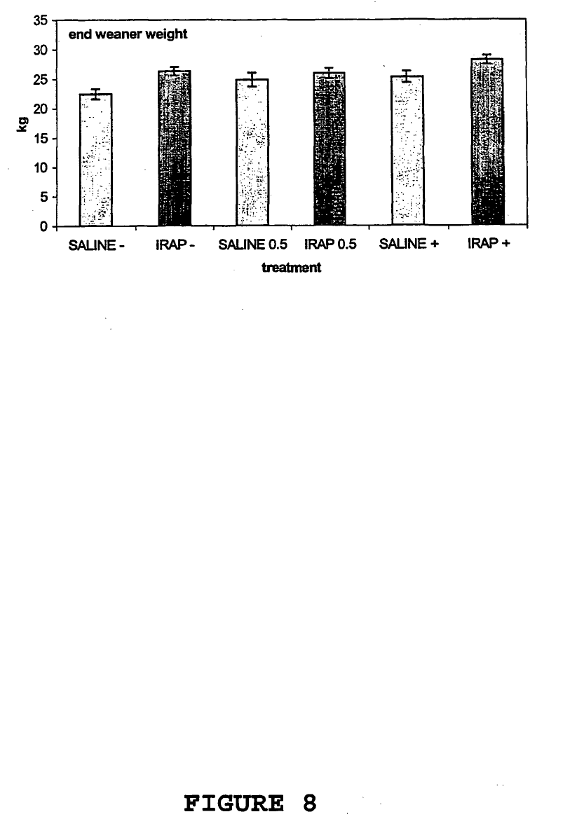

At the end of the weaner phase, pigs treated with IL-1ra weighed more than their saline treated counterparts at all 3 levels of medication (FIG. 8). Significantly, the weight of weaner pigs treated with IL-1ra without antibiotic medication was greater than the weight of weaner pigs treated with saline and provided normal levels of antibiotics (Irap− vs Saline+, FIG. 8). As expected, antibiotic medication enhanced the growth of saline treated pigs as well as improving the growth of pigs treated with IL-1ra.

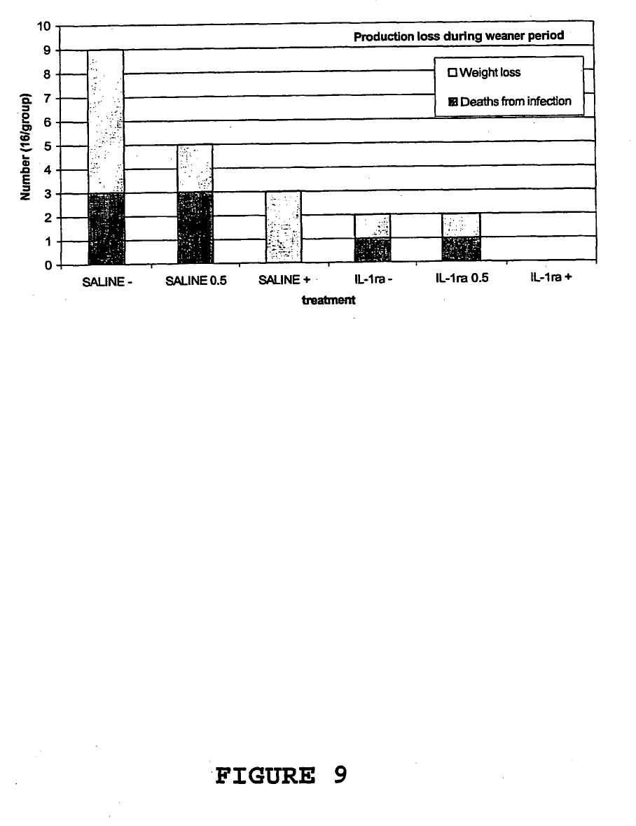

The presence of antibiotic medication had a considerable effect on the health of pigs during the weaner phase as measured by the number of pigs experiencing weight loss or mortality (FIG. 9). Increased levels of antibiotics decreased the number of pigs experiencing weight loss or mortality in saline treated pigs. However, pigs treated with IL-1ra showed the least recordings of weight loss or mortality regardless of antibiotic levels. In the absence of antibiotics, over half of the 16 pigs treated with saline showed decreased health and production over the weaner period. However, this number was reduced to 2 out of 16 pigs with IL-1ra treatment (FIG. 9).

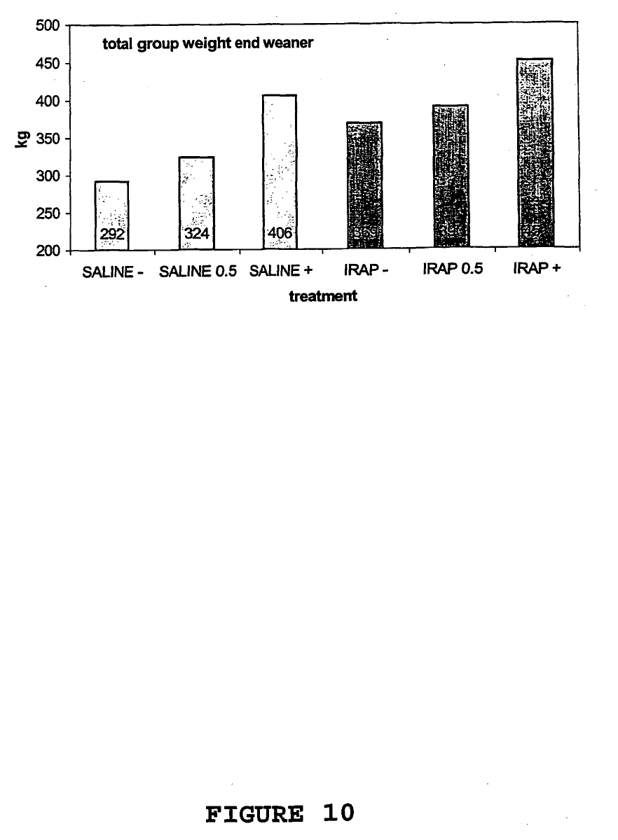

This improvement in health and decreased mortality over the weaner period, combined with improved weight gain resulted in marked differences for total group weights at the end of the weaner phase (FIG. 10). Saline treated groups weighed 292 kg, 324 kg and 406 kg with no medication, reduced medication and full medication respectively. Comparable total group weights at the end of the weaner phase for IL-1ra treated pigs were 369 kg, 390 kg and 451 kg. This increase in group weight with IL-1ra treatment reflects an increase in productivity of 26.4%, 20.4% and 11.1% at zero, reduced and normal levels of antibiotic administration respectively.

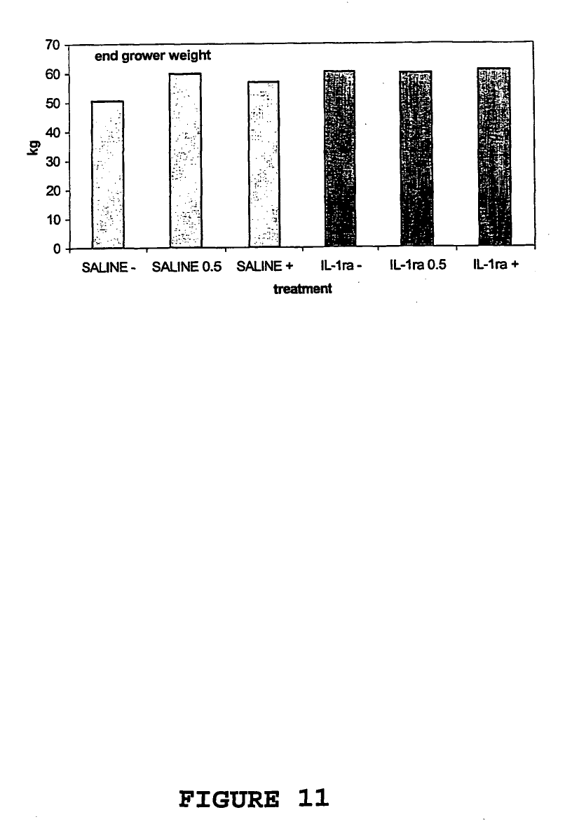

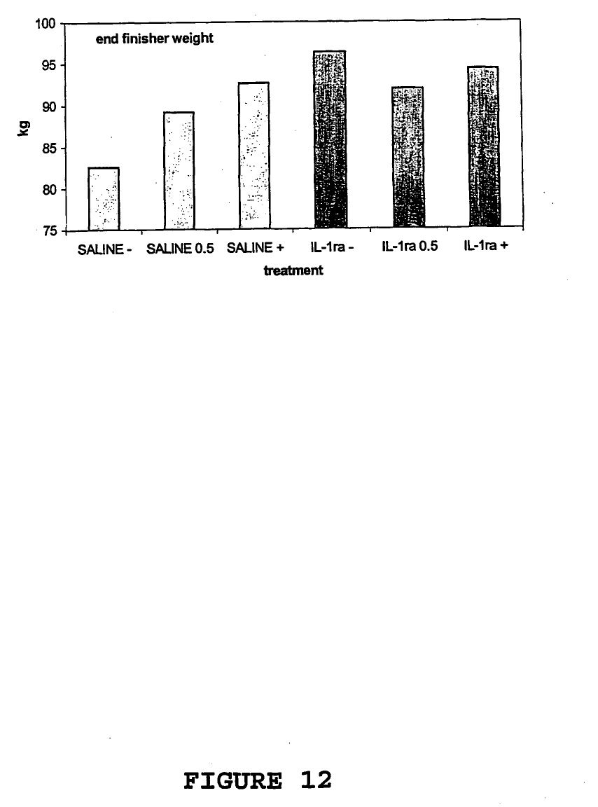

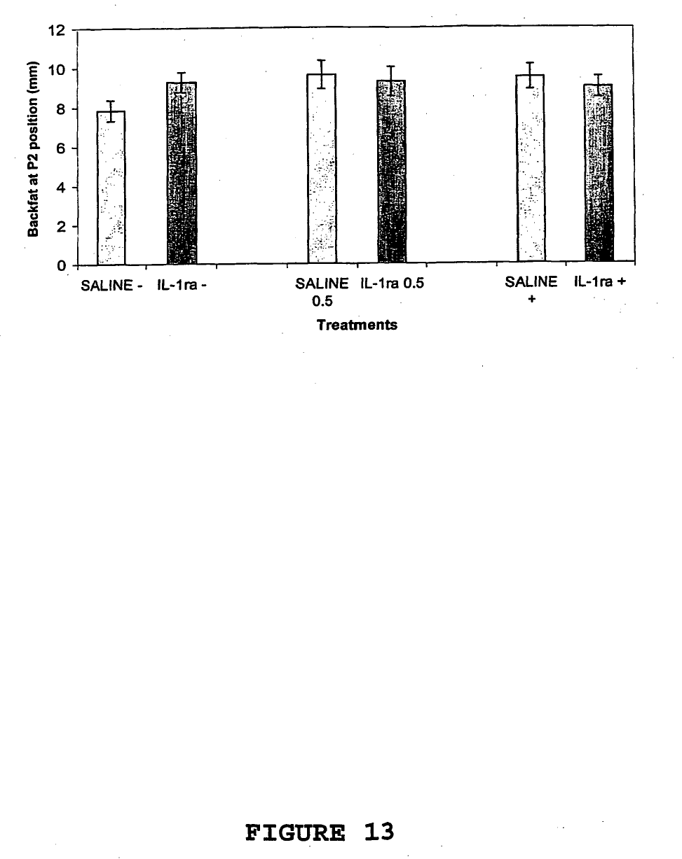

The improvement in productivity seen in the weaner phase with IL-1ra treatment was continued throughout the grower and finisher phases (FIGS. 11 and 12). At the end of the grower phase, pigs treated with IL-1ra were heavier than all the saline treatment regardless of medication level (FIG. 11). Importantly, delivery of IL-1ra in the absence of antibiotics resulted in higher weights at the end of the grower phase than the current industry level of medication. Finishing weight in saline treated pigs was affected by medication, in a dose-dependent manner (FIG. 12), with increasing levels of medication resulting in increased weight at slaughter. However, this pattern was not repeated in the IL-1ra treated pigs. Pigs treated with IL-1ra weighed 16.7% more than saline treated pigs in the absence of antibiotic medication. Indeed, IL-1ra administration without medication outperformed the current industry level of antibiotic treatment in the promotion of growth in finisher pigs (FIG. 12). These results suggest that IL-1ra is more efficacious than antibiotic medication in promoting growth in pigs throughout the production phases. Although IL-1ra treatment resulted in larger pigs at slaughter, this increase in weight was not accompanied by an increase in P₂ backfat (FIG. 13). Pigs treated with IL-1ra in the absence of antibiotics had P2 backfat levels that were comparable to those of pigs treated with saline and provided full antibiotic medication (FIG. 13). Such results indicate that the improvement in liveweight seen with IL-1ra administration is carried through to the end product, resulting in leaner carcasses.

Conclusions

1). IL-1ra improved growth in pigs in the absence of in-feed antibiotics, and with reduced levels of antibiotics.

2). The improvement in growth produced by IL-1ra treatment exceeded that seen by the current industry level of antibiotic medication.

3). IL-1ra improved growth throughout the production phases from weaner through to finisher, resulting in higher weights at slaughter.

4). IL-1ra improved the health of weaner pigs as seen by reduced mortalities and reduced incidence of weight losses compared with saline treatment.

5). The improvement in health parameters seen with IL-1ra administration was greater than that provided by antibiotic medication.

6). Continuous administration of IL-1ra did not have any deleterious effects on pigs.

7). IL-1ra improved weight without compromising carcass quality as seen by unchanged P₂ backfat values at slaughter.

### EXAMPLE 3

### Delivery of Recombinant IL-1RA to Improve the Health of Weaner Pigs—Infected with Haemorrhagic E. coli

This study determined whether IL-1ra was able to improve the health of pigs exposed to infections, such as haemorrhagic E. coli. A further aim was to determine whether IL-1ra could improve growth in pigs infected with E. coli at weaning. It was also designed to show whether IL-1ra could reduce infection rates and improve health in pigs infected with E. coli. Finally it was hoped that the experiments would assess the prophylactic or therapeutic potential of IL-1ra for E. coli infections in weaner pigs compared to current antibiotic treatments.

Experiment Design

Male weaner pigs, with a mean weight of 5.4 kg were allocated to groups of 8, with the mean weight being equalised between groups. Pigs were housed in group pens containing a replicate from each treatment group. Pigs were provided with pelleted feed and water ad libitum.

Pigs were treated with recombinant saline, IL-1ra or the antibiotic, Apralan, and challenged with E. coli according to the schedule outlined in Table 3. Saline, or 200 μg of IL-1ra were delivered intramuscularly in 1 ml doses. Apralan was delivered orally according to manufacturer's instructions at a dose of 12 mg/kg. E. coli challenges were delivered orally in an 8 ml dose containing 10⁸ cfu/ml. Blood was sampled from pigs by venipuncture at −2 days, day 0, and +6 days from initial challenge with E. coli as outlined in Table 3. Blood was assayed for immunological parameters including white blood cell number, differential cell counts, lymphocyte subset enumeration, IgG levels and cytokine production. Pigs were weighed at day −2 and at the end of the trial on day 6.

Faecal samples were taken from each pig daily for 5 days from day 2 to day 6 after challenge; these samples were cultured on sheep blood agar to quantify E. coli load. Growth on sheep blood agar was scored from 0 to 5 (where 0 was no growth, 1 signified growth in the primary inoculum, 2 signified growth in the first streak, 3 signified growth in the 2^{nd} streak, 4 signified growth in the 3^{rd} streak, and 5 signified growth of E. coli in the final streak). The condition of faeces was noted as normal, wet or diarrhoea, as an indication of clinical signs.

At the conclusion of the experiment on day 7, pigs were euthanased and swabs were taken from different areas in the gastro-intestinal tract, including the small intestine (25%, 50% and 75% along the length of the small intestine), the caecum and colon, and from the faeces in situ. These post-mortem swabs were cultured on sheep blood agar to quantify E. coli load as described above.

TABLE 3
EXPERIMENTAL PROTOCOL TO EXAMINE THE
EFFICACY OF IL-1RA AS A PROPHYLACTIC
FOR E. coli INFECTION IN PIGS

| DAY OF TRIAL | EVENT |
|---|---|
| −2 | Blood sample |
| | IL-1ra injection |
| | Weights |
| −1 | IL-1ra injection |
| | Apralan orally |
| 0 | E. coli challenge |
| | IL-1ra injection |
| | Apralan orally |
| | Blood sample |
| 1 | E. coli challenge |
| | IL-1ra injection |
| | Apralan orally |
| 2 | E. coli challenge |
| | IL-1ra injection |
| | Apralan orally |
| | Faecal swabs |
| 3 | E. coli challenge |
| | Apralan orally |
| | Faecal swabs |
| 4 | Faecal swabs |
| 5 | Faecal swabs |
| 6 | Blood sample |
| | Faecal swabs |
| | Weights |
| 7 | Euthanase |
| | Swabs from gut and faeces |

Results

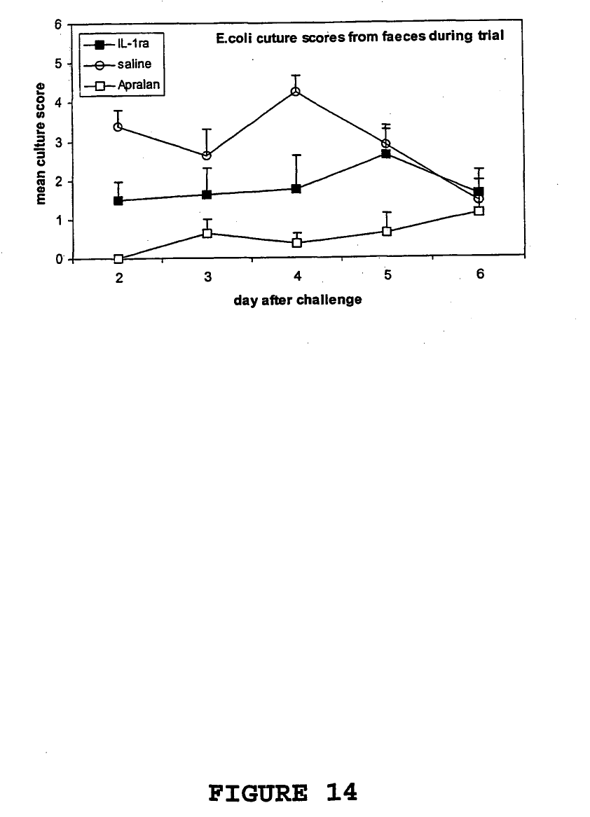

Pigs treated with IL-1ra or Apralan showed decreased E. coli shedding in faeces compared to control pigs treated with saline (FIG. 14). Pigs treated with Apralan had reduced bacterial shedding from day. 2 to day 5 after challenge, while IL-1ra treated pigs had reduced bacterial shedding from day 2 through to day 4. On day 6 after challenge, bacterial shedding from all groups was equal. For both Apralan and IL-1ra treatments, E. coli shedding in faeces returned to saline control levels 3 days after the final treatment dose was delivered. Overall, the Apralan treated group displayed the least bacterial shedding of all treatments.

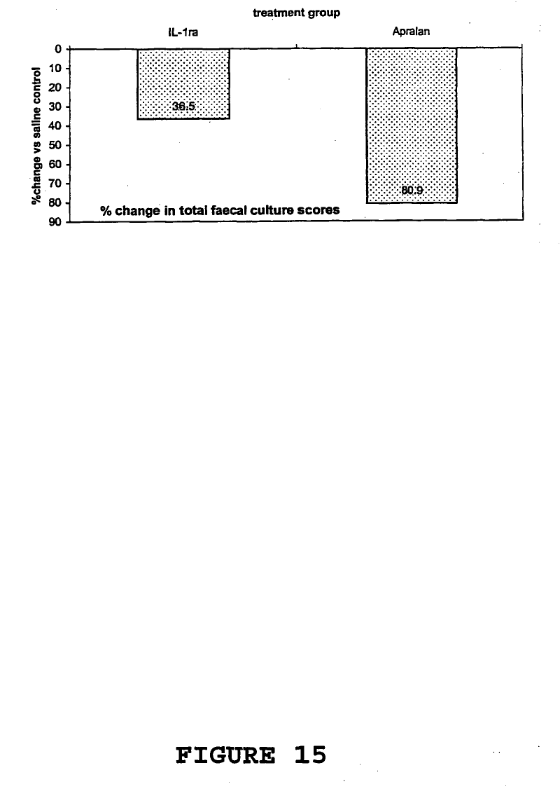

Faecal scores tallied over the entire challenge period for each group show an 80.9% decrease in faecal shedding for Apralan treated pigs compared to saline treated controls, while IL-1ra treated pigs showed a 37% reduction in bacterial shedding compared to saline treated controls (FIG. 15).

In commercial situations, reduced bacterial shedding from infected pigs would further reduce cross-infection of other members of the herd or pen, thereby improving the health of weaners, and therefore growth. Enhancing the health and growth of weaner pigs would result in improved productivity in later phases since the major predictor of productivity is weight at the end of the weaner phase.

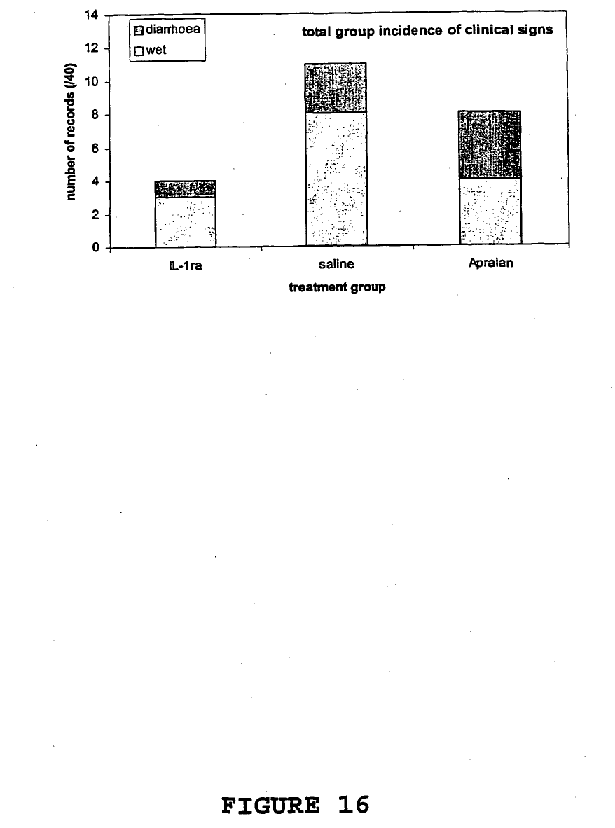

Clinical signs, recorded as the changes in faecal condition such as the presence of wet faeces or diarrhoea, were decreased in pigs treated with IL-1ra or Apralan (FIG. 16). Pigs treated with IL-1ra had fewer recorded cases of wet faeces and diarrhoea than saline controls or Apralan treated pigs. Pigs treated with Apralan had fewer recordings of wet faeces than did saline controls, but also displayed a minor increase in the prevalence of diarrhoea in the post-challenge period (FIG. 16).

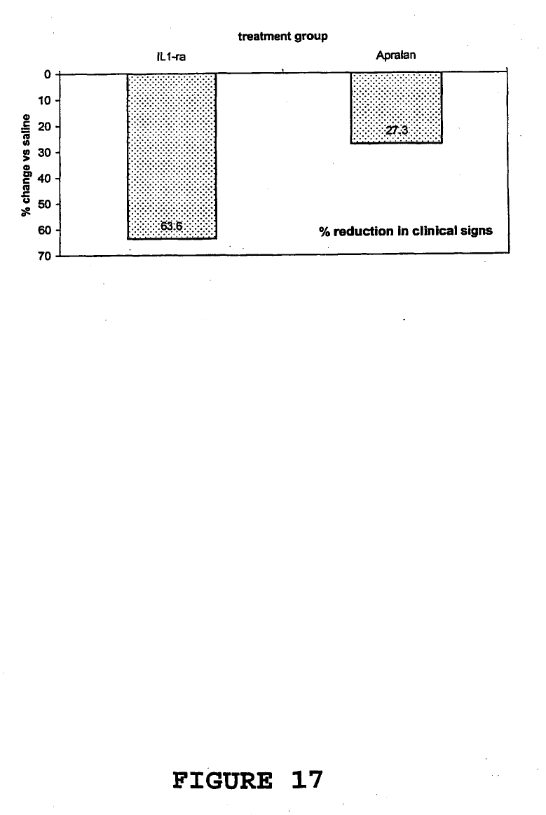

When these clinical signs are described as a percentage reduction in symptoms compared to saline controls, IL-1ra treatment produced a 64% reduction in clinical signs, while Apralan caused clinical symptoms to be reduced by 27% (FIG. 17).

The results for clinical symptoms show that IL-1ra and Apralan were both able to reduce the outward signs of infection with E. coli. In this measure of health, IL-1ra out-performed Apralan, the current antibiotic treatment for E. coli infections.

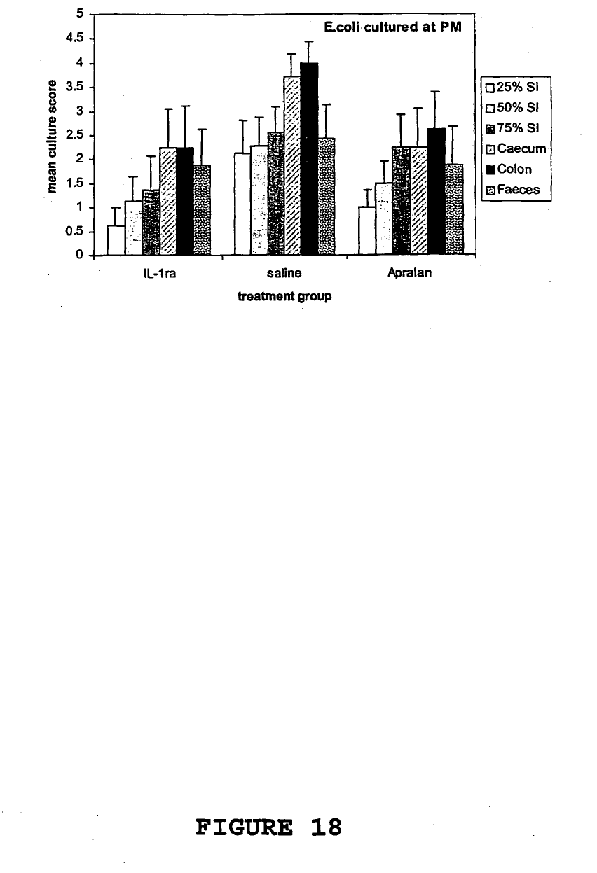

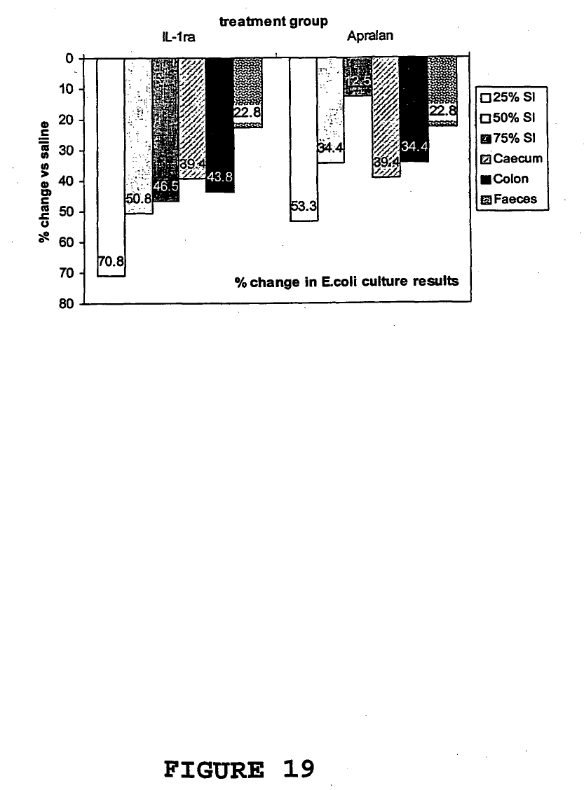

Both Apralan and IL-1ra treatments resulted in reduced bacterial load in all areas of the gastro-intestinal tract (GIT) compared with saline-treated controls (FIG. 18). Pigs treated with IL-1ra recorded the lowest culture scores for all areas sampled in the small intestine and the colon. IL-1ra and Apralan treated pigs had equally low faecal culture scores for samples taken from the caecum and faeces. IL-1ra treatment resulted in reductions of 71% E. coli in the anterior part of the small intestine, 51% reduction in the mid small intestine, 47% in the posterior small intestine, 39% in the caecum, 44% in the colon and 23% in faeces in situ compared to saline treated controls (FIG. 19).

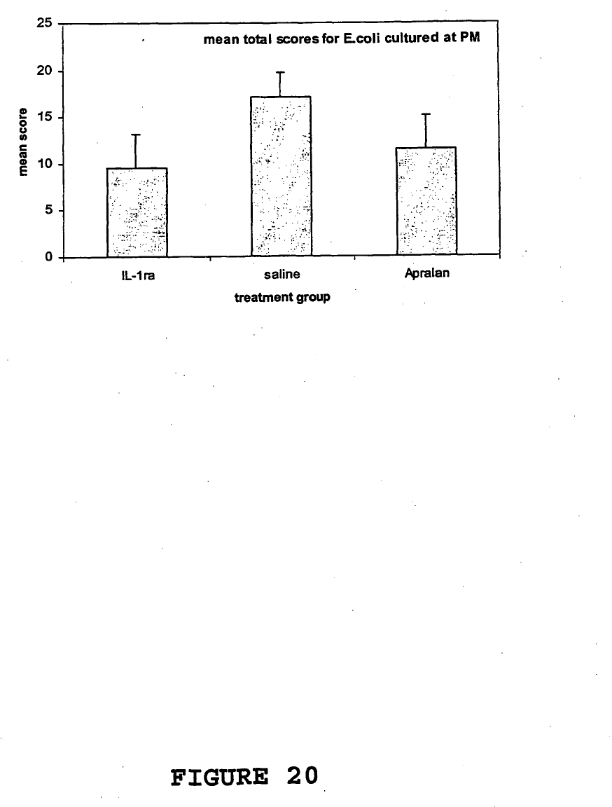

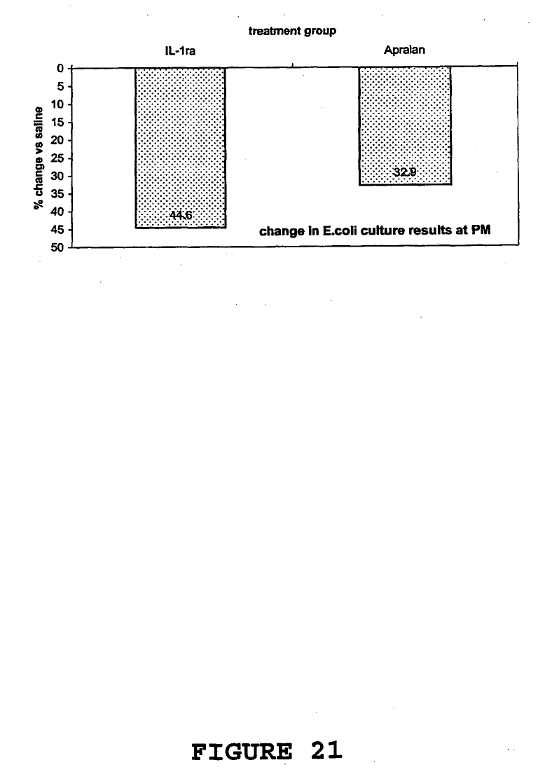

When all culture scores were tallied for each pig and used to calculate group mean total scores (FIG. 20), pigs treated with IL-1ra scored less than 10 out of a possible 30, compared with 17/30 for saline treated pigs, and 12/30 for Apralan treated pigs. When this data is expressed as a percentage reduction in E. coli culture scores compared to saline controls (FIG. 21), prophylactic application of IL-1ra resulted in a 45% reduction in the amount of E. coli in the gastro-intestinal tract. Treatment with Apralan was only able to reduce E. coli colonisation in the gut by 33%.

These results illustrate that bacterial load was lowest in pigs treated with IL-1ra, further emphasising the value of this preparation for the control of haemorrhagic E. coli in young pigs.

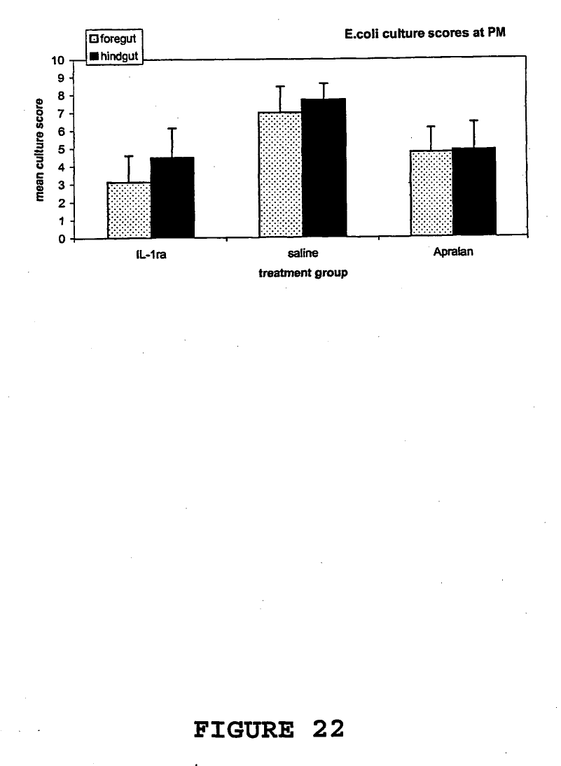

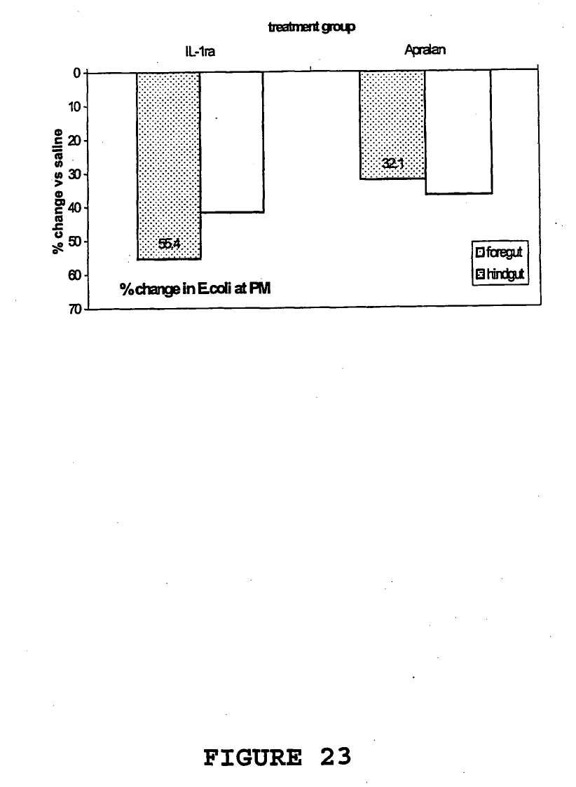

When the post-mortem results for E. coli cultures were separated on the basis of location in the gut, differences may be seen in the action of IL-1ra and Apralan (FIG. 22). E. coli bacterial load in the small intestine (foregut) correlates with the severity of disease, as the small intestine is the site in which the secretory diarrhoea is manifested. Treatment with IL-1ra reduced the bacterial load in the small intestine by 55% compared with saline controls, while Apralan caused a reduction of 32% in the bacterial load in the small intestine (FIG. 23). In the hindgut area (caecum and colon), bacterial loads recorded for Apralan and IL-1ra treatments were similar, resulting in a 37% and 42% reduction in E. coli respectively, compared to saline controls (FIG. 23).

The ability of IL-1ra to reduce bacterial load preferentially in the foregut would suggest that this treatment may reduce the severity of disease associated with haemorrhagic E. coli infection. Indeed, these results support those recorded for clinical signs of disease where pigs treated with IL-1ra had reduced incidence of diarrhoea compared to other treatments (FIG. 16). Thus, IL-1ra may be a potential replacement or adjunct for the antibiotics currently administered in the pig industry to control the deleterious effects of this disease on pig production.

A summary of the comparative effects of IL-1ra and Apralan on bacterial shedding, clinical signs and bacterial load at post-mortem is included in Table 4.

TABLE 4
SUMMARY COMPARING THE THERAPEUTIC EFFECTS
OF IRAP (IL-1RA) AND APRALAN FOR THE
CONTROL OF HAEMORRHAGIC E. coli INFECTIONS
IN YOUNG WEANER PIGS

| | | |
|---|---|---|
| Change compared to | | |
| saline treated controls | Apralan | IL-1ra |

| | | | | |
|---|---|---|---|---|
| Presence of bacterial | ↓ | 4 days | ↓ | 3 days |
| shedding in faeces | | | | |
| Change on faecal | ↓↓ | 80.8% | ↓ | 36.5% |
| bacterial load | | | | |
| Change in clinical | ↓ | 27.3% | ↓↓ | 63.6% |
| signs | | | | |
| E. coli at post-mortem | ↓ | 32.9% | ↓↓ | 44.6% |
| E. coli in foregut | ↓ | 32.1% | ↓↓ | 55.4% |
| E. coli in hindgut | ↓ | 36.8% | ↓ | 41.7% |

Conclusions

1). IL-1ra improved the health of pigs i.e. it reduced the clinical signs of disease, in terms of faecal changes associated with haemorrhagic diarrhoea in the presence of haemorrhagic E. coli infection.

2). The improvement in health produced by IL-1ra treatment was equal to, and in some cases, greater than that produced by treatment with the antibiotic Apralan, the current method of treating haemorrhagic E. coli in pigs.

3). IL-1ra treatment resulted in decreased bacterial shedding in faeces during the course of infection compared with saline-treated controls. Pigs treated with IL-1ra showed bacterial shedding significantly less than saline treated controls on 3/5 days after challenge. Such results suggest that under commercial conditions decreasing the bacterial load in the environment may reduce infection rates.

4). The effect of IL-1ra administration resulted in decreased numbers of bacteria in all areas of the GIT compared with saline treated controls.

5). Significantly, IL-1ra caused a 55% reduction in the bacterial load in the small intestine (foregut), the site in which secretory diarrhoea is normally located during the course of E. coli infection. As bacterial load in the small intestine is associated with disease severity, IL-1ra may have a significant therapeutic effect on the progression and pathology of the disease.

6). IL-1ra treatment outperformed Apralan, the current antibiotic treatment used in industry, in reducing clinical signs of disease, E. coli levels present in the gut at post-mortem, in addition to E. coli present in the crucial site of the small intestine.

### EXAMPLE 4

### Delivery of Recombinant IL-1RA to Improve the Health and Productivity of Weaner Pigs Infected with an Enteric Inflammatory Pathogen Causing Swine Dysentery, Brachyspira (Serpulina) hyodysenteriae

The aim of this example was to determine whether IL-1ra could improve the health of pigs infected with an enteric inflammatory pathogen causing swine dysentery, Brachyspira (Serpulina) hyodysenteriae. A further aim was to determine whether IL-4 could improve the growth rate of pigs under conditions of challenge with swine dysentery.

Experiment Design

Male pigs with a mean starting weight of 6.5 kg, were allocated to treatment groups consisting of eight pigs. Pigs were housed in group pens, with each pen containing a replicate from each of the treatment groups. One group of 8 pigs was housed in a separate room and left uninfected to act as untreated controls. Pigs were provided with pelleted feed and water ad libitum.

Prior to swine dysentery challenge, pigs were treated with 200 μg recombinant IL-1ra or 1 ml saline via intramuscular injection. Lincocin was delivered as a 2 ml intramuscular injection according to the manufacturer's instructions. Cytokines, antibiotics and challenges were performed at intervals described in the experimental protocol outlined in Table 5. Pigs were infected with Brachyspira hyodysenteriae at day 0, day 1 and day 2, given as an oral bolus of 120 ml of spirochaete culture in log phase of growth, containing approximately 10⁸ cells.

Faecal swabs and blood samples were taken from each pig at intervals described in Table 5. Faecal swabs were cultured for the presence of spirochaetes. Blood samples were assayed for immunological parameters as described in Example 3 above. Pigs were weighed at weekly intervals throughout the experiment, which was terminated by euthanasia on days 19 and 20 after the initial challenge. At post-mortem, swabs from areas of the hindgut were cultured for the presence of spirochaetes, and the gross pathological condition of the gastro-intestinal tissue noted.

TABLE 5
PROTOCOL FOR EXPERIMENTAL PROCEDURES TO ASSESS
THE EFFICACY OF IL-1RA AS A PROPHYLACTIC TREATMENT
FOR SWINE DYSENTERY INFECTION

| | | | | | Inject 1 ml | Inject | |
|---|---|---|---|---|---|---|---|
| | | Faecal | | | IL-1ra, or | 2 ml | |
| Day | Weigh | Swabs | Infect | Bleed | Saline | Lincocin | Kill |
| −7 | X | | | | | | |
| −6 | | | | | | | |
| −5 | | | | | | | |
| −4 | | | | | | | |
| −3 | | | | | | | |
| −2 | | | | | X | | |
| −1 | | | | | X | | |
| 0 | X | X | X | X | X | | |
| 1 | | | X | | X | X | |
| 2 | | | X | | X | X | |
| 3 | | | | | | X | |
| 4 | | | | | | | |
| 5 | | X | | X | X | X | |
| 6 | | | | | | X | |
| 7 | X | X | | | X | X | |
| 8 | | | | | | X | |
| 9 | | X | | | X | X | |
| 10 | | | | | | | |
| 11 | | | | | | | |
| 12 | | X | | X | X | X | |
| 13 | | | | | | X | |
| 14 | X | X | | | X | X | |
| 15 | | | | | | X | |
| 16 | | X | | | X | X | |
| 17 | | | | | | | |
| 18 | | | | | X | X | |
| 19 | X | | | | | | X |
| 20 | X | | | | | | X |

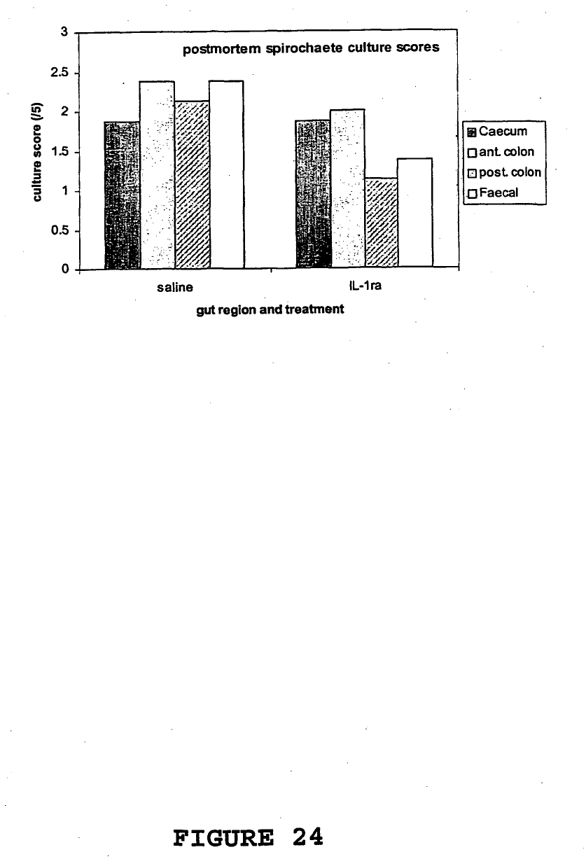

Spirochaete cultures taken from the hindgut at post mortem show that treatment of pigs with IL-1ra reduced the number of spirochaetes residing in the gut compared to saline controls (FIG. 24). IL-1ra was able to reduce spirochaete culture scores in the anterior colon, posterior colon and faeces compared to saline treated controls. As expected, pigs that were not challenged with swine dysentery did not have spirochaetes in their hindgut or faeces at post mortem (data not shown).

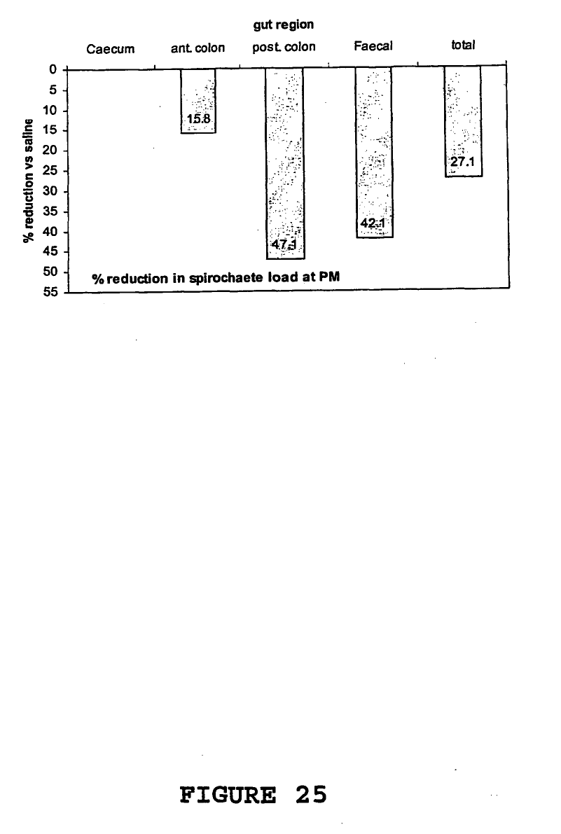

Compared to saline treated pigs, IL-1ra treatment resulted in a 15.8% reduction in the anterior colon, 47.1% in the posterior colon and 42.1% reduction in faecal spirochaetes (FIG. 25). The net effect of IL-1ra treatment was a 27% reduction in spirochaetes throughout the GIT.

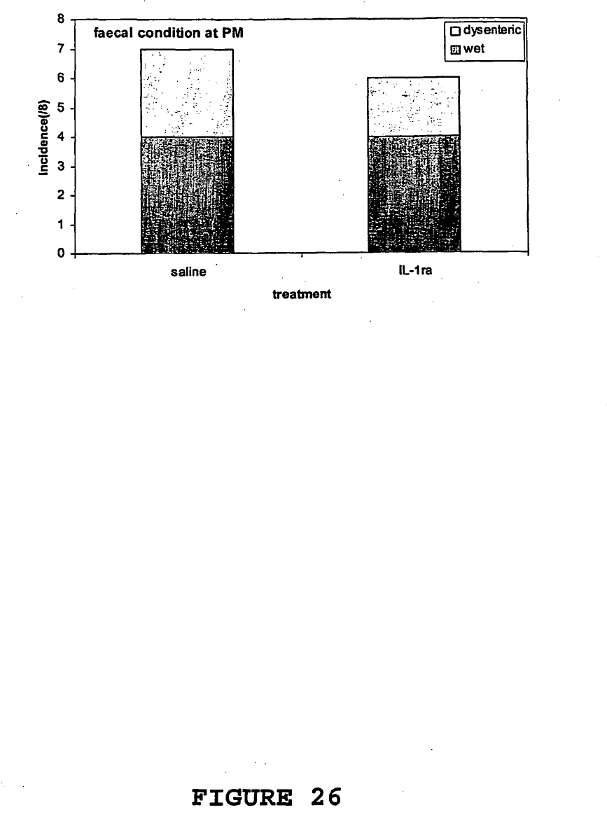

In addition to a reduction in the number of spirochaetes in the gut, treatment with IL-1ra also reduced the clinical signs associated with infection indicated by faecal condition. FIG. 26 shows that IL-1ra treated pigs showed fewer signs of dysentery-affected faeces (wet and mucoid with blood) compared to saline treated controls.

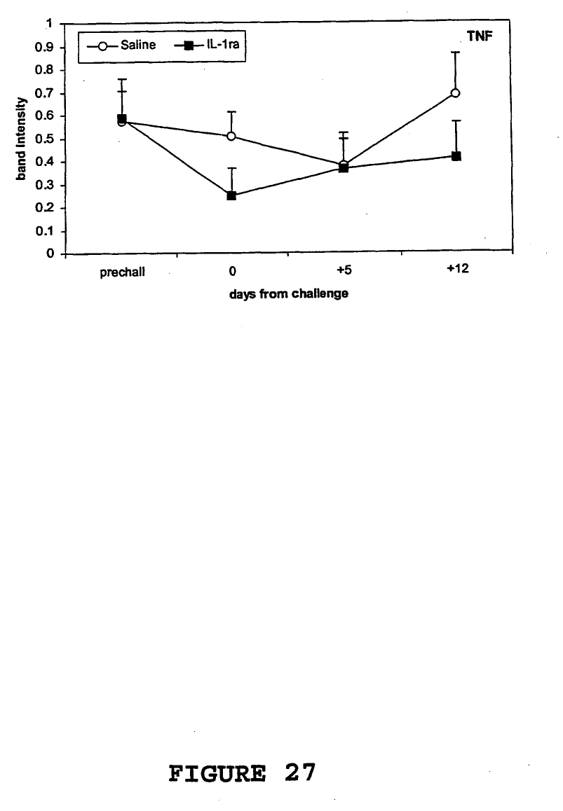

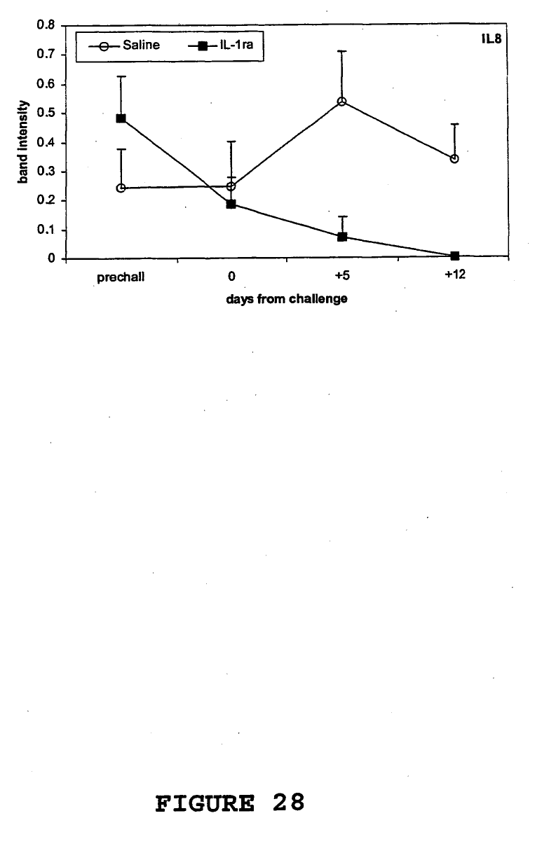

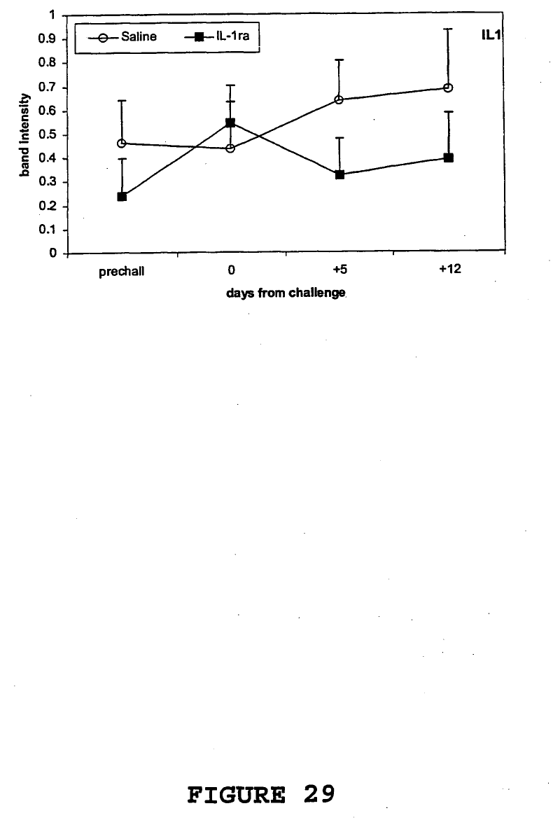

Treatment with IL-1ra was able to reduce the production of the pro-inflammatory cytokines TNF, IL-8 and IL-1 (FIGS. 27, 28 and 29) compared to saline treated controls. Importantly, pro-inflammatory cytokines are associated with sickness behaviour in animals and have been implicated in reduced productivity seen in intensively housed livestock. This anti-inflammatory ability of IL-1ra may translate to long term improvements in growth. Indeed, such results have been described for Examples 1, 2 and 3 above.

Furthermore, the clinical manifestation of swine dysentery is a chronic inflammatory pathology presumably exacerbated by inflammatory mediators such as pro-inflammatory cytokines. The ability of IL-1ra to reduce the production of these inflammatory mediators may play a role in reducing the pathology associated with swine dysentery infection.

Such results confirmed that IL-1ra was able to reduce the deleterious effect of swine dysentery infection on the health of pigs. IL-1ra was known to have anti-inflammatory effect on the immune system, thus, a reduction in inflammatory pathological changes in the gut associated with dysentery may be attributable to both the anti-inflammatory properties of this cytokine and a reduced spirochaete load (as seen in FIG. 24).

Conclusions

1). Treatment of pigs with IL-1ra reduced the number of spirochaetes present in the hindgut and faeces at post-mortem compared with saline treatment.

2). IL-1ra reduced the clinical manifestation of swine dysentery infection as detected by faecal condition, compared with saline controls.

3). Treatment of pigs with IL-1ra resulted in reduced production of pro-inflammatory cytokines which are associated with impaired growth and productivity.

4). IL-1ra has been shown to improve the health of pigs in two enteric infection models: haemolytic E. coli and Brachyspira (Serpulina) hyodysenteriae (swine dysentery). Improvements in health in both models were described by reduced clinical symptoms during infection. In the E. coli model, the improvement in health was accompanied by a reduction in infection associated pathology at post-mortem. In the swine dysentery model, reduced levels of pro-inflammatory cytokines were also noted. The ability of prophylactic treatment with IL-1ra to improve the health of pigs exposed to E. coli was comparable to the performance of the current industry standards of antibiotic treatment. Thus, IL-1ra has potential as an alternative, or supplement with, treatment to antibiotics, or preventative, for E. coli and swine dysentery in pigs. The potential of IL-1ra as a health promoter may be further enhanced by concurrent application with antibiotic therapeutics.

### EXAMPLE 5

### Delivery of Plasmids and Recombinant IL-1RA to Improve Growth and Health in Pigs Infected with Actinobacillus pleuropneumoniae

The aim of this example was to determine whether IL-1ra could improve the health of pigs infected with the inflammatory lung pathogen, Actinobacillus pleuropneumoniae (App). Furthermore, another aim was to determine whether IL-1ra could improve the growth rate of pigs under conditions of challenge with App. Additionally, this example aimed to determine whether plasmid DNA or recombinant delivery of IL-1ra was more efficacious.

Experiment Design

Male pigs, with a mean starting weight of 52 kg, were allocated to 5 treatment groups as outlined in Table 6. Pigs were housed in group pens, with each pen containing a replicate from each of the treatment groups. The starting weights of each treatment group and each pen were equal prior to the start of the trial. Pigs were provided with pelleted feed and water ad libitum.

Recombinant IL-1ra and saline-were administered as 1 ml doses, given subcutaneously behind the ear. Plasmids were administered in 1 ml doses, given intramuscularly in the hind-leg. Flunix was administered as a 2 ml dose according to the manufacturer's instructions, and delivered intramuscularly in the neck. The timetable of administration is outlined in Table 6 below.

TABLE 6
TREATMENTS AND DOSES APPLIED IN CYTOKINE
EXPERIMENT (N = 4 PER GROUP) TO
ASSESS THE EFFICACY OF IL-1RA AS A PROPHYLACTIC
TREATMENT ACTINOBACILLUS PLEUROPNEUMONIAE
INFECTION

| TREATMENT | TREATMENT DOSE | |
|---|---|---|
| Saline | 2 | ml |
| Flunix | 2.2 | mg/kg |
| IL-1ra | 100 | μg |
| Plasmid control | 100 | μg |
| Plasmid IL-1ra | 100 | μg |

Prior to challenge, pigs were treated with recombinant cytokines, flunix or plasmids as described Table 7. Pigs were anaesthetised and infected intratracheally with 7.5×10⁵ pfu App on day 0.

Blood was sampled from pigs by venipuncture at 0, 24 h and 14 days post-challenge. Blood was assayed for immunological parameters as previously described. Pigs were weighed weekly from delivery of plasmids and for 2 weeks after challenge.

TABLE 7
PROTOCOL FOR EXPERIMENTAL PROCEDURES TO ASSESS
THE EFFICACY OF IL-4 AS A PROPHYLACTIC TREATMENT
Actinobacillus pleuropneumoniae INFECTION

| EVENT | TIMING OF ADMINISTRATION |
|---|---|
| Plasmid delivery | −10 days |
| Recombinant delivery | −2 days and day 0 |
| Challenge | Day 0 |
| Clinical visits | 30 post-challenge |

Results

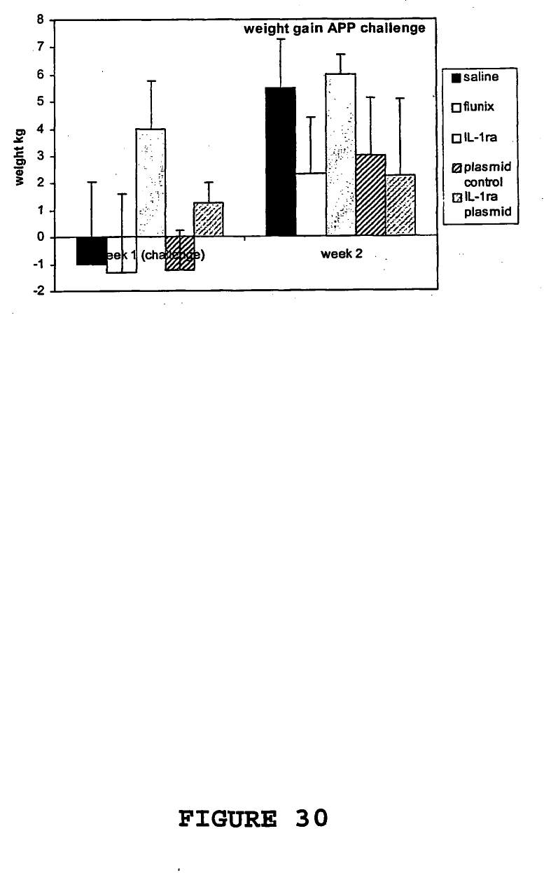

During the week of challenge, IL-1ra improved the growth of pigs (FIG. 30) compared to saline-treated controls. Pigs treated with saline, flunix (a non-steroidal anti-inflammatory drug, NSAID), or control plasmid showed weight loss, while pigs treated with IL-1ra or plasmid IL-1ra showed positive growth during the week of challenge. In the week following challenge, all groups of pigs gained weight, but again, recombinant IL-1ra treated pigs gained more weight on average than pigs in other treatment groups. Pigs treated with saline recovered significantly in the second week after challenge, while pigs treated with IL-1ra continued to gain weight. Pigs treated with plasmids or flunix had the poorest growth of all groups in the second week of challenge.

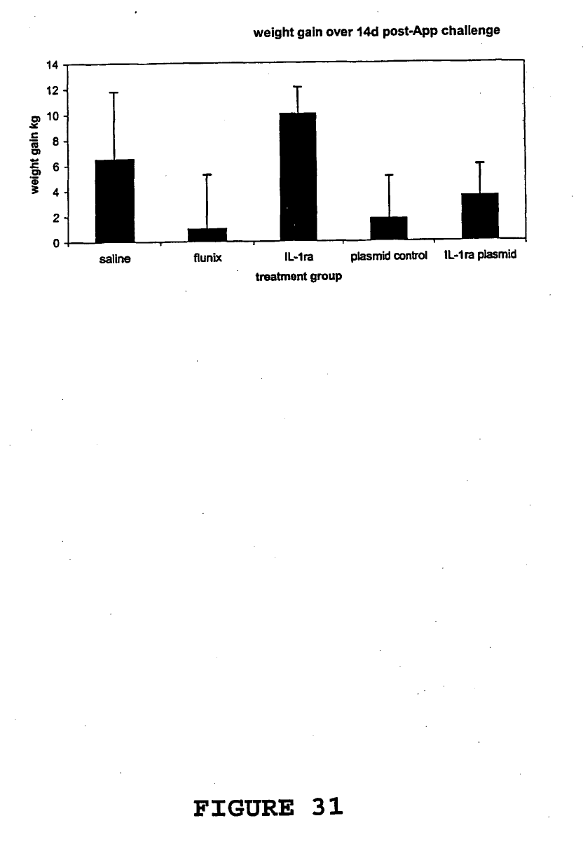

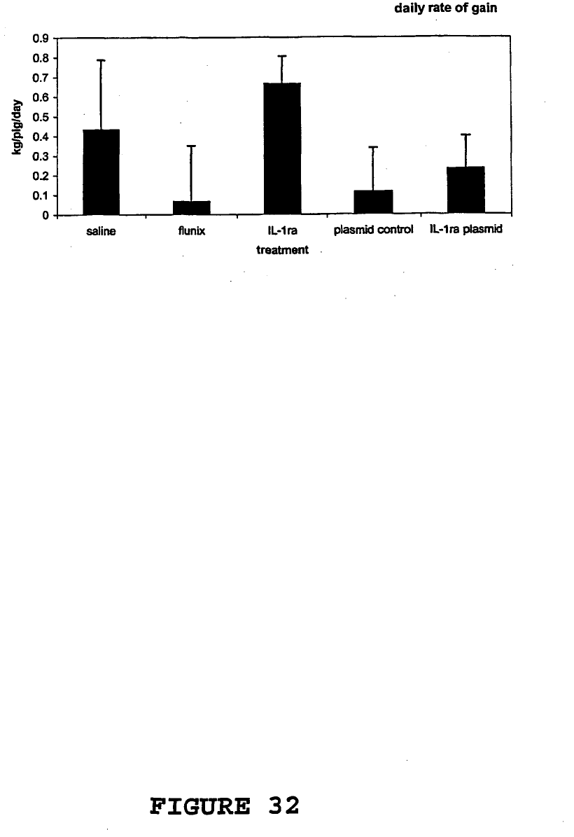

Weight gain over the 2 week period following challenge with App (FIG. 31) showed that recombinant IL-1ra treatment increased weight gain compared to saline-treated controls, although this result was not statistically significant. Flunix and plasmid-control were the poorest performing treatments in terms of growth, compared to saline-treated controls. IL-1ra plasmid performed better than the plasmid control group in terms of growth over the 2 week period after challenge (FIG. 31). Similar patterns of performance were noted for daily rate of gain (FIG. 32), with pigs treated with recombinant IL-1ra gaining on average 667 g per day compared to 433 g per day for saline treated control pigs.

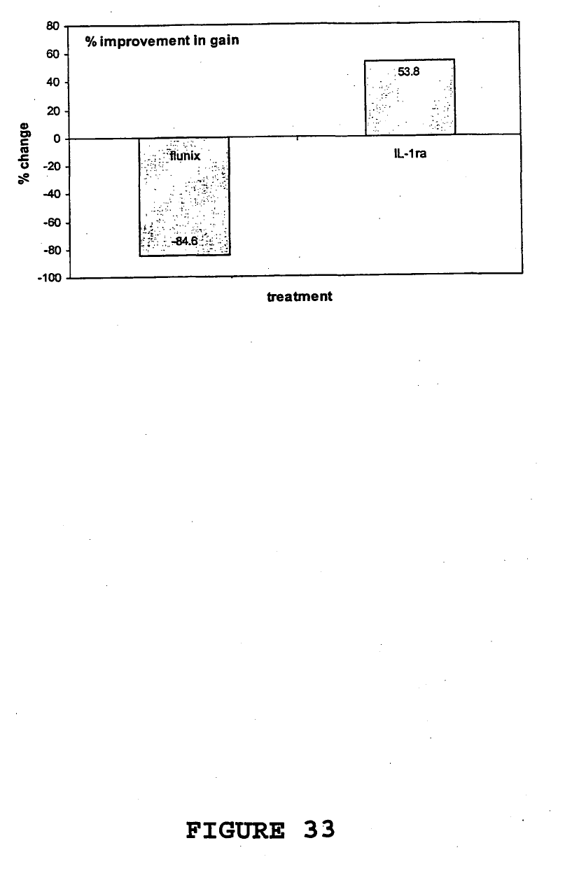

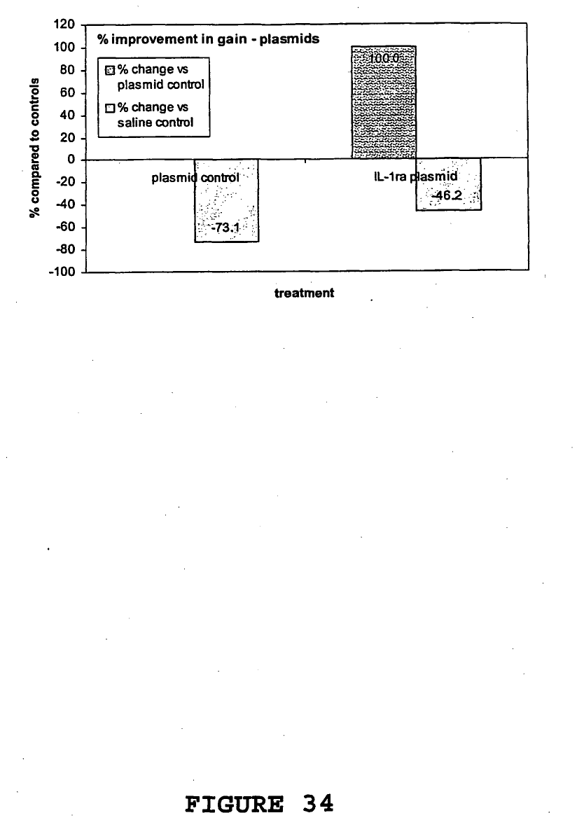

Treatment with recombinant IL-1ra resulted in improvements in weight gain of 53.8% over saline controls, while treatment with the NSAID flunix caused an 84.6% reduction in weight gain (FIG. 33). Plasmid treatments generally had lower weight gain than did saline controls however, the IL-1ra plasmid improved weight gain by 100% compared to its plasmid control (FIG. 34).

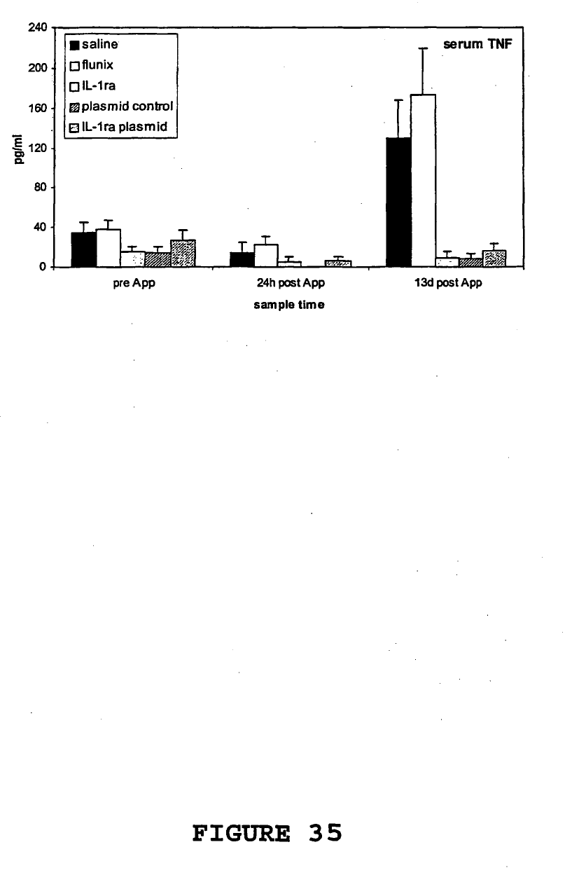

Pro-inflammatory cytokines, TNFα and IL-6 were elevated in several groups after challenge with App. Interestingly, the NSAID flunix, failed to inhibit the production of TNFα (FIG. 35), which may help to explain the poor growth seen in this group. Recombinant IL-1ra, plasmid control and IL-1ra plasmid all had reduced levels of TNFα at day 13 after challenge compared with pre-challenge levels. These 3 treatments also had significantly lower levels of TNFα production than saline-treated and flunix-treated pigs at day 13 after challenge (p<0.05).

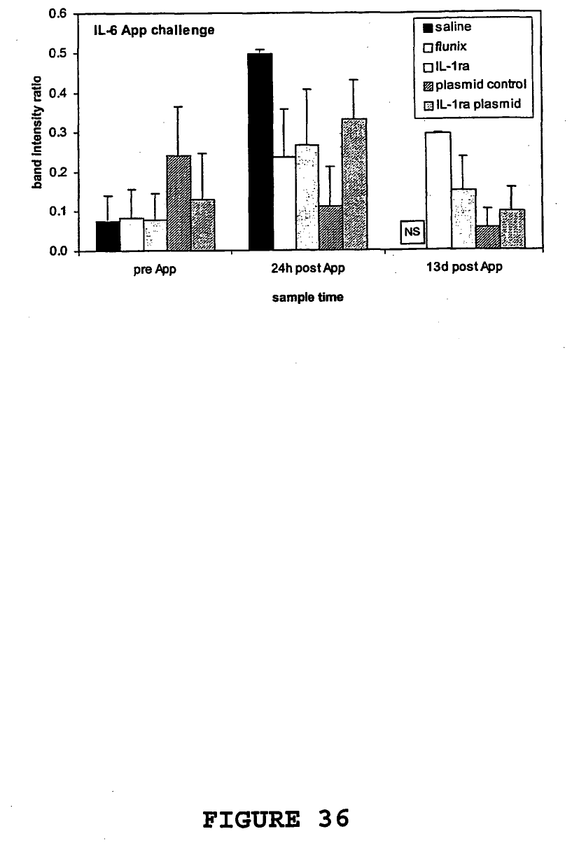

All treatments reduced the production of IL-6 24 h after challenge compared with saline treated controls (FIG. 36), and this trend continued until 13d post-challenge. Unfortunately, IL-6 data was not retrievable for the saline treatment at 13 days after App challenge due to sampling error. After 13 days of challenge, pigs treated with IL1-ra as either plasmid or recombinant had reduced levels of the pro-inflammatory cytokine, IL-6, compared to pigs treated with flunix.

While the anti-inflammatory cytokine treatments did cause reductions in the levels of pro-inflammatory cytokines in the circulation, and in some cases improved growth, the relationship between pro-inflammatory cytokines and impaired growth is still unclear. Generally, groups of pigs with reduced levels of pro-inflammatory cytokines were the groups that also had the least inhibition of growth in the first week after challenge. Further work is required to elucidate the mechanism of weight loss in pigs under this challenge model.

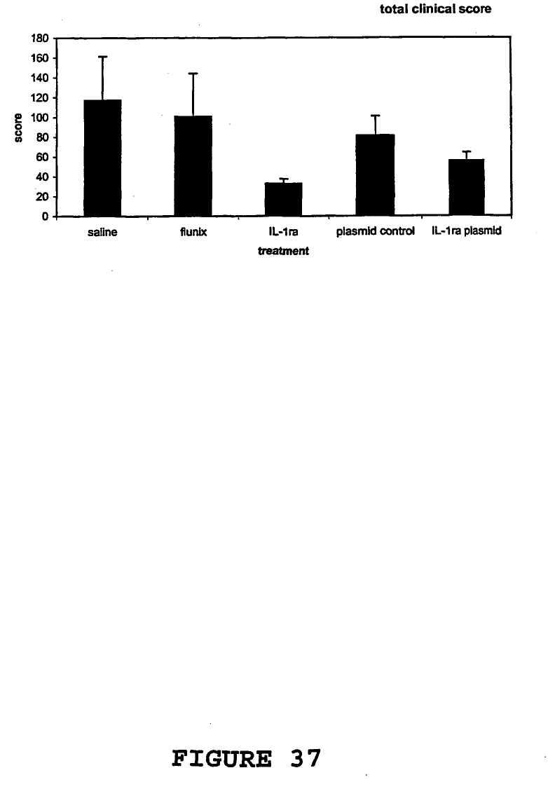

In addition to improving the growth of pigs, we found that cytokine treatment could improve the health of pigs exposed to App challenge. The data in FIG. 37 shows the mean clinical scores over 30 visits conducted during the first week of challenge. The severity of symptoms displayed by each pig, such as lethargy, coughing and breathing parameters was scored from 0-8, and pigs which died or were euthanased were arbitrarily given a score of 8 at each subsequent visit. Pigs treated with recombinant IL-1ra had significantly reduced clinical signs of disease compared to saline-treated controls (p<0.05, FIG. 37). IL-1ra delivered as a plasmid also resulted in reduced clinical symptoms compared to saline and plasmid control pigs. Pigs treated with either saline or flunix showed the greatest clinical signs of App disease of all treatment groups.

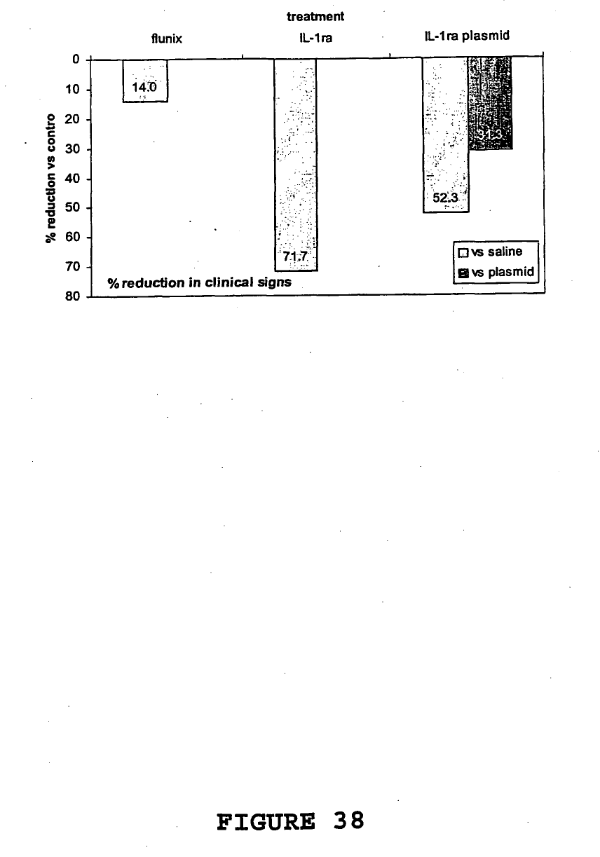

IL-1ra delivered as a recombinant caused a reduction of 72% in the presence of clinical symptoms compared to pigs treated with saline (FIG. 38). IL-1ra delivered in plasmid form produced a reduction of 52% compared to saline-treated controls, and 31% reduction compared to plasmid-treated controls (FIG. 38). IL-1ra delivered as plasmid or recombinant was more effective than flunix in reducing the clinical symptoms of App infection.

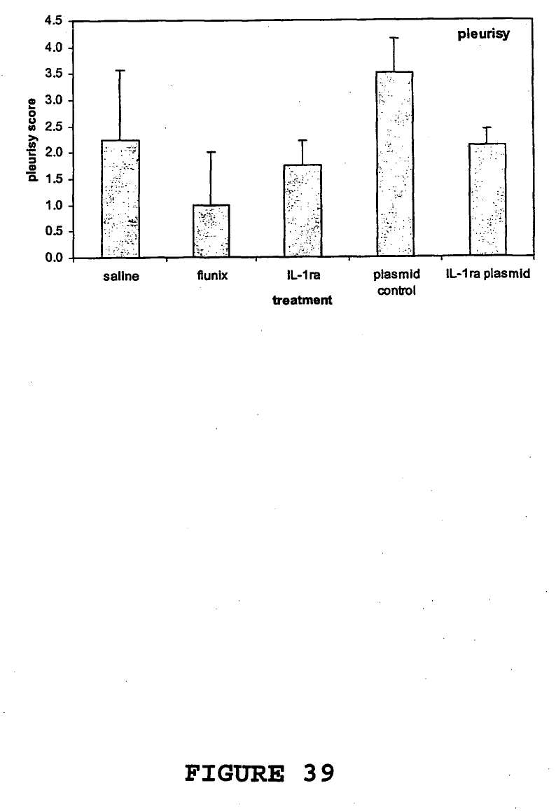

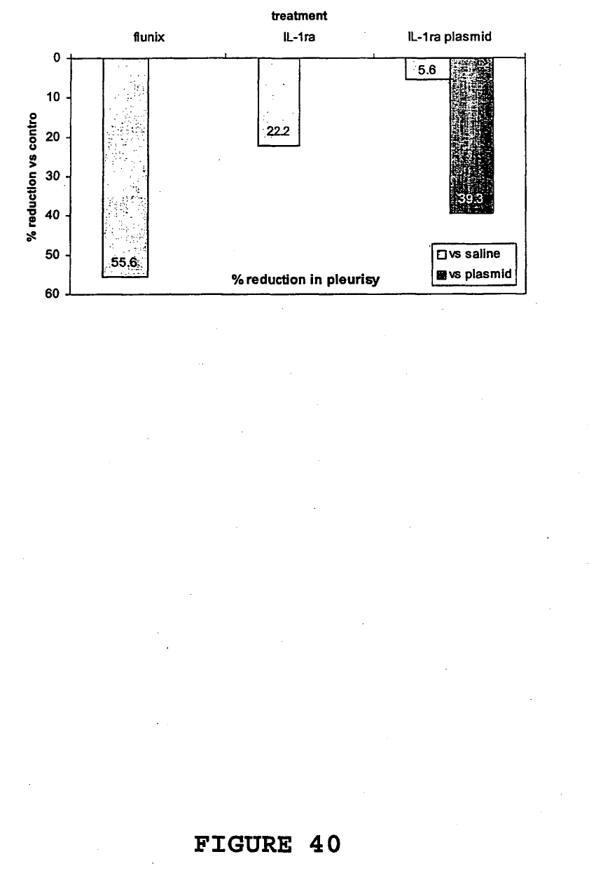

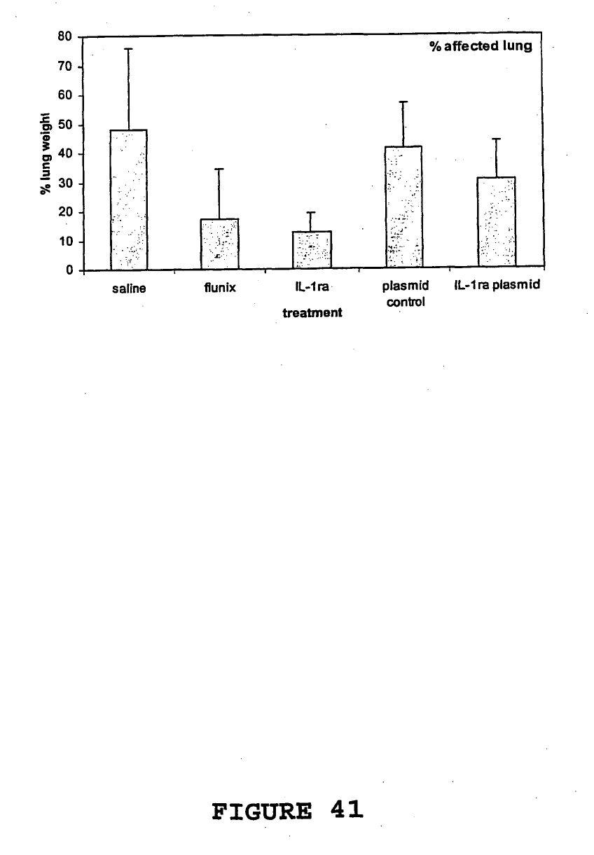

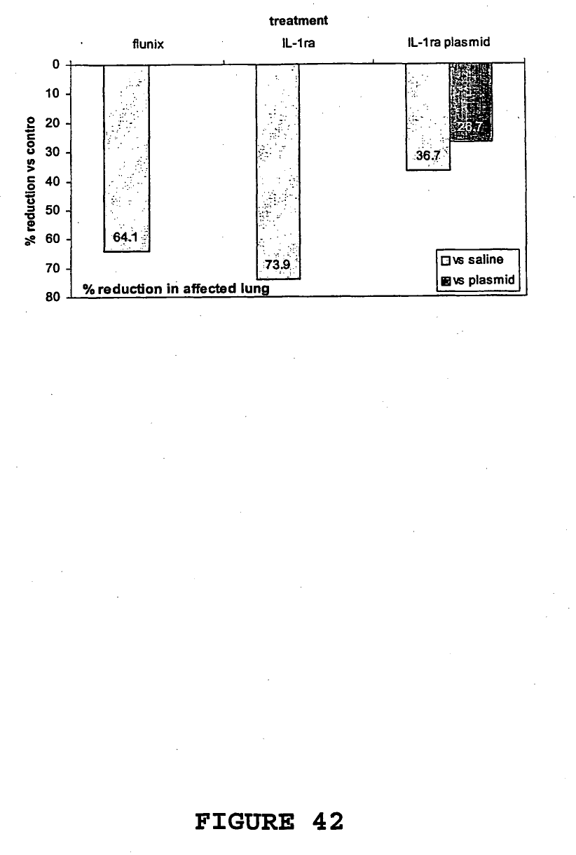

At the conclusion of the trial, pigs were euthanased and the lungs removed for post-mortem examination. Lungs were scored for pleurisy from 0-5 (FIGS. 39 and 40) and the degree of pleuropneumonia was determined by weighing affected lung and expressed as a percentage of total lung weight (FIGS. 41 and 42). Pigs treated with flunix and IL-1ra had less pleurisy than the saline controls (FIG. 39). Although pigs treated with IL-1ra delivered as plasmid had less pleurisy than their plasmid-treated controls, their level of pleurisy was comparable to that of saline-treated controls (FIG. 39). Recombinant IL-1ra reduced the levels of pleurisy by 22% compared to saline treated controls, while treatment with flunix reduced pleurisy by 55.6% (FIG. 40). IL-1ra delivered as plasmid reduced pleurisy by 5.6% compared to saline treated controls, and 39.3% compared to plasmid controls (FIG. 40).

The percentage of lung affected by App lesions was greatly reduced in pigs treated with either flunix or recombinant IL-1ra compared with saline-treated controls (FIG. 41). Both the saline control group, and the plasmid control group had similar levels of lesion-affected lung. IL-1ra plasmid reduced the percentage of lung affected by App lesions when compared to saline and plasmid control groups, but the ability of plasmid IL-1ra to impair the pathology of App disease is not as pronounced as IL-1ra delivered as a recombinant or flunix (FIG. 41). These results reflect a reduction in affected lung mass of 73.9% for IL-1ra, 64.1% for flunix and 36.7% for plasmid IL-1ra compared to saline treated controls (FIG. 42).

Conclusions

1). Recombinant IL-1ra was able to greatly increase the growth of pigs compared to saline treated controls during the first week of App challenge. Pigs treated with IL-1ra were subsequently 4 kg heavier at the termination of the experiment, after 2 weeks of challenge than their saline treated peers, which represents an improvement in growth of 69%. Pigs treated with flunix had the lowest growth over the 2 week challenge period.

2). Recombinant IL-1ra, plasmid control and plasmid IL-1ra were able to reduce the production of the pro-inflammatory cytokines TNFα and IL-6 which are associated with poor growth performance. Flunix was able to reduce the production of IL-6 only.

3). IL-1ra greatly reduced the severity of clinical symptoms of disease during the challenge, as did IL-1ra delivered as plasmid. Recombinant IL-1ra reduced clinical symptoms by 72%, while plasmid IL-1ra reduced clinical signs by 52% compared to saline treatment.

4). Flunix was able to reduce the level of pleurisy seen at post-mortem. IL-1ra reduced pleurisy by 22% compared to saline treatment.

5). Flunix, IL-1ra and plasmid IL-1ra all reduced the percentage of lung affected by App lesions. Treatment

Blood was sampled from pigs by venipuncture at −24 h, +0 h, +24 h and +3 weeks from challenge. Blood was assayed for immunological parameters as previously described. Pigs were weighed at day −1, day 10 and at 3 weeks.

TABLE 8
TREATMENTS AND DOSES APPLIED IN CYTOKINE EXPERIMENT
(N = 8 PER GROUP) TO ASSESS THE EFFICACY
OF DIFFERENT DOSES OF IL-1RA AND IL-1RA + IL-4
AS A PROPHYLACTIC TREATMENTS
Actinobacillus pleuropneumoniae INFECTION

| TREATMENT | TREATMENT DOSE |
|---|---|
| saline | 2 ml |
| IL-1ra lo | 2 μg/kg |
| IL-1ra hi | 10/kg |
| Synergy lo | 2 μg/kg IL-4 + 2 μg/kg |
| | IL-1ra |
| Synergy hi | 10 μg/kg IL-4 + 10 μg/kg |
| | IL-1ra |

Results

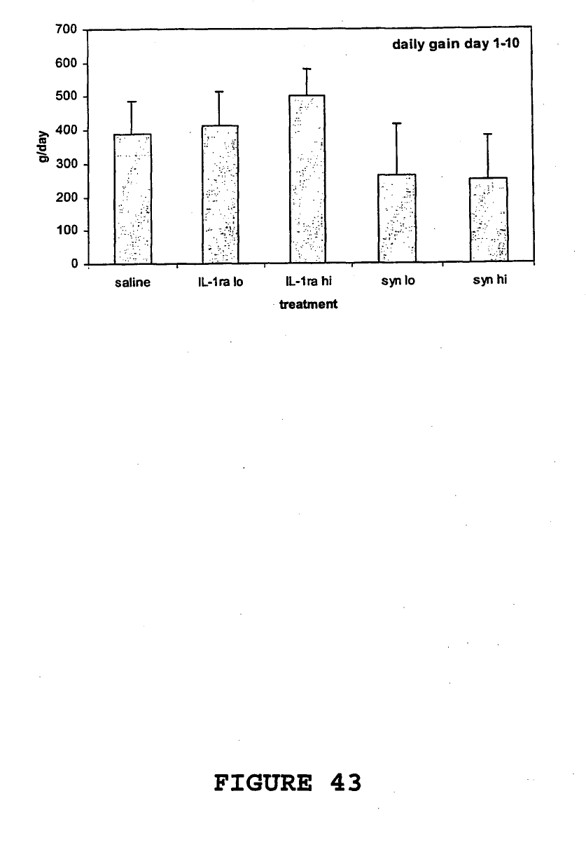

Unlike Example 5, animals treated with saline did not experience weight loss during the early stages of challenge with App (compare FIG. 30 with FIG. 43). Despite this result, improvement in growth was seen in pigs treated with the high dose of IL-1ra (FIG. 43), equivalent to an increase in rate of gain in excess of 100 g/day. Application of both IL-1ra and IL-4 together to investigate synergy resulted in a depressed growth response during the first 10 days of App challenge compared to saline treated pigs. Variation within groups was high, accounting for large error bars and lack of statistical significance in this instance. However, the trends of improved weight gain with IL-1ra seen in Example 5 were repeated in this experiment.

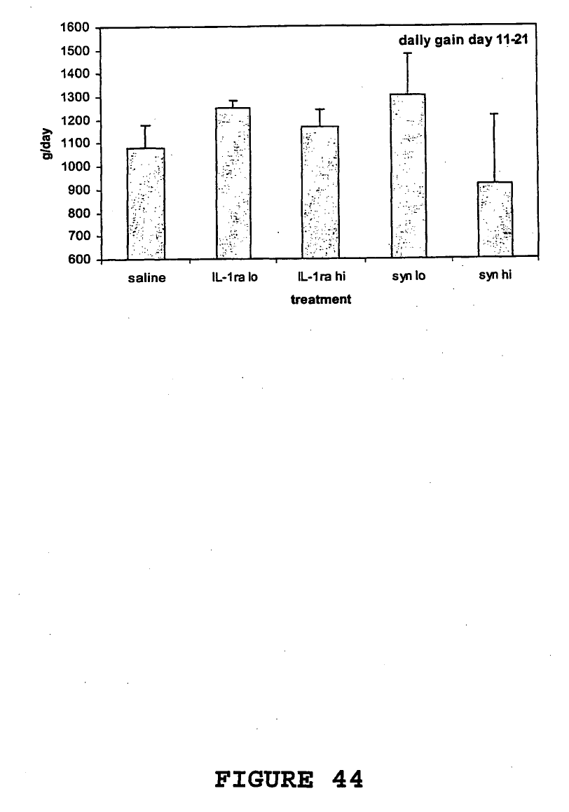

In the last 10 days of challenge, pigs treated with low doses of IL-1ra or low doses of IL1ra+IL-4 showed the greatest rate of gain (FIG. 44) at 1250 g/day and 1306 g/day respectively, compared to 1079 g/day for saline treated controls. Pigs treated with high doses of IL-1ra gained 1170 g/day, which was higher than the rate of gain for saline treatment. The high synergy dose resulted in lower weight gain during the latter stages of App challenge.

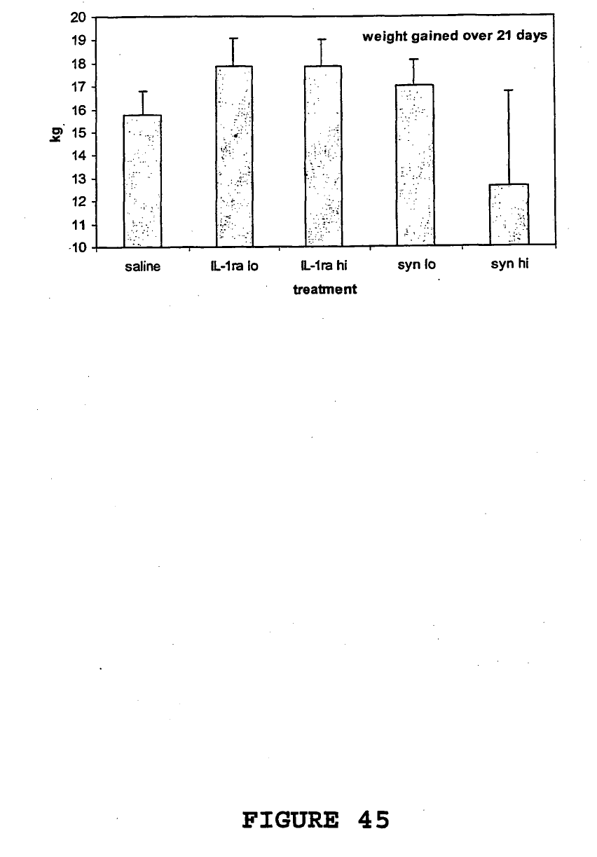

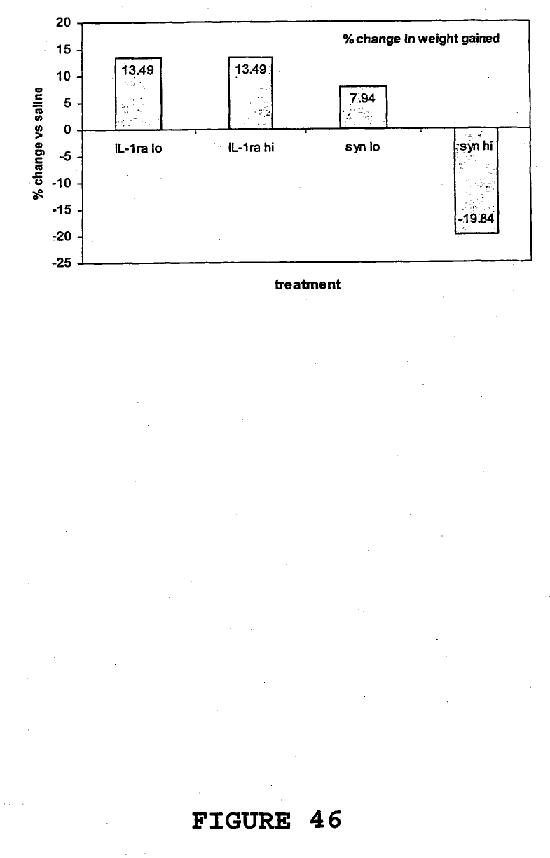

Pigs treated with IL-1ra at low or high doses and low dose IL-1ra+IL-4 exhibited higher weight gain than saline treated controls (FIG. 45). During the 21-day challenge period, pigs treated with low and high doses of IL-1ra, or low dose synergy treatment gained 17.9 kg and 17 kg respectively, while saline treated controls gained only 15.75 kg (FIG. 45). Thus, treatment with IL-1ra or low dose IL-1ra+IL-4 improved growth by 13.5% and 7.9% respectively (FIG. 46) compared to saline treated controls.

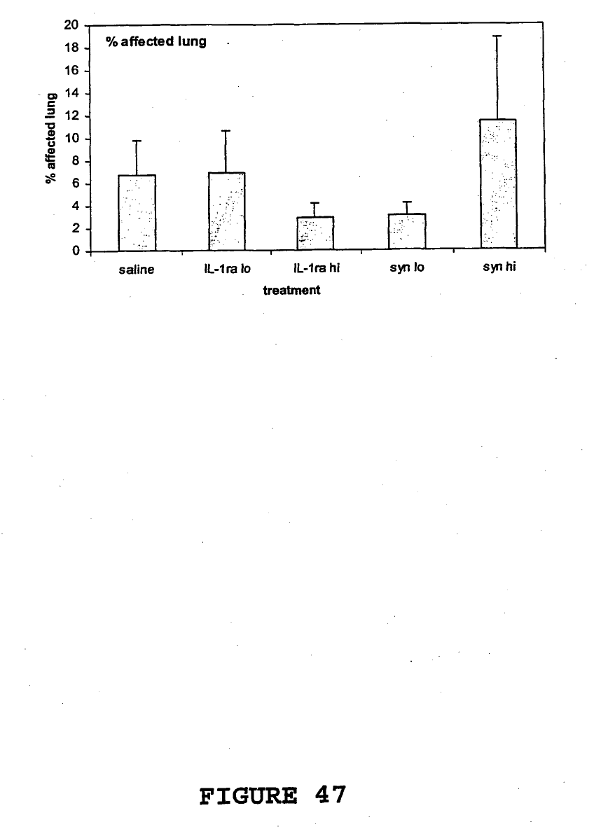

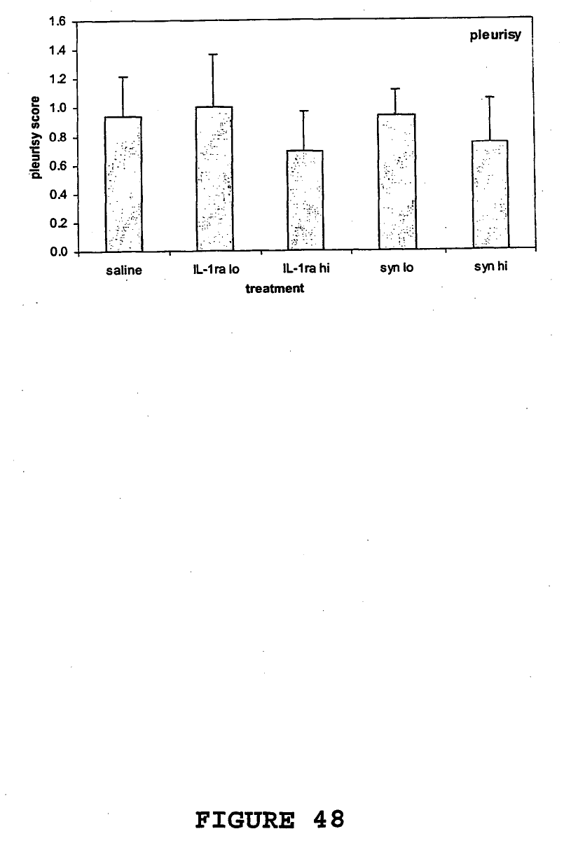

As seen in Example 5, treatment of pigs with recombinant IL-1ra caused reduced disease severity as recorded in pathology results at post-mortem (FIGS. 39, 40, 41 and 42). In the current example, application of IL-1ra at the high dose reduced the amount of lung affected by App lesions (FIG. 47). Similarly, delivery of low dose IL-1ra and IL-4 combines also reduced affected lung weight compared to saline treatment. The degree of pleurisy seen at post-mortem was reduced with high doses of IL-1ra and high doses of the synergy treatment (FIG. 48).

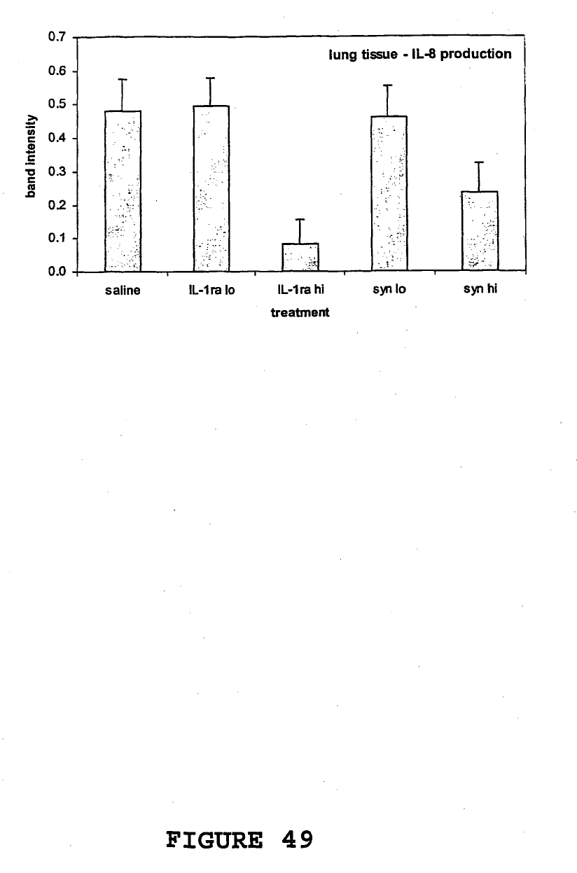

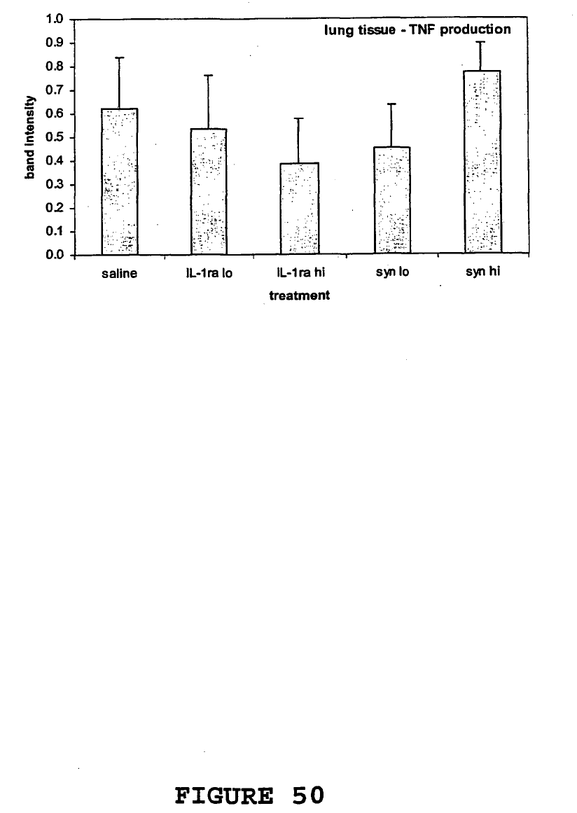

Production of the pro-inflammatory cytokine IL-8 was greatly reduced with high dose treatment of IL-1ra and IL-1ra+IL-4 (FIG. 49) compared to other treatments. IL-8 recruits neutrophils to the lung and subsequent neutrophil degranulation is suspected to be a major factor in the pathology of App infection. Thus, reduction of IL-8 levels in lung tissue is likely to result in decreased pathology and improved health in pigs exposed to App infection. Similarly, the production of another pro-inflammatory cytokine, TNFα, was inhibited in lung tissue by treatment with high doses of IL-1ra or low doses of IL-1ra+IL-4 (FIG. 50). These results suggest that an anti-inflammatory mechanism may play a role in the beneficial effects of these treatments on the growth and health of pigs under conditions of App challenge.

Conclusions

1). IL-1ra at high doses improved growth early in challenge, while IL-1ra at low or high doses, and low dose IL-1ra+IL-4 improved growth in the latter stages of challenge.

2). IL-1ra at low or high doses, and low doses of IL-1ra+IL-4 resulted in increased weight gain over the entire challenge period.

3). IL-1ra at high doses and low dose IL-1ra+IL-4 reduced the amount of lung affected by App lesions. IL-1ra and IL-1ra+IL-4 at high doses reduced pleurisy scores.

4). IL-1ra and IL-1ra+IL-4 at high doses had an anti-inflammatory effect as noted by reduced production of pro-inflammatory cytokines in lung tissue.

5). High doses of IL-1ra significantly decreased the production of IL-8 in the lungs, which is associated with pathology

6). These results support the results of Example 5 which found an improvement in growth and a reduction in pathology with IL-1ra therapy given prior to and at the time of infection with App.

### EXAMPLE 7

### Therapeutic Delivery of Recombinant IL-1RA at Low and High Doses to Improve the Health and Growth of Pigs Infected with Actinobacillus pleuropneumoniae

The aim of this example was to determine whether therapeutic delivery of IL-1ra after Actinobacillus pleuropneumoniae (App) infection was established, could abrogate infection and improve growth in pigs.

Experiment Design

Male pigs with a mean starting weight of 34.6 kg, were allocated to 4 treatment groups of 9 pigs each. Treatments were saline, IL-1ra at 2 μg per kg, IL-1ra at 10 μg per kg and Excenel, the current clinical treatment for App infection. Pigs were housed in pens of 3 pigs, with 3 replicates of each. Pigs were provided with pelleted feed and water ad libitum. The starting weights of each treatment group and each pen were equal prior to the start of the trial.

Pigs were anaesthetised and infected intratracheally with 7.5×10⁵ pfu on day 0. Recombinant IL-1ra and saline were administered as 2ml doses, given subcutaneously behind the ear. Excenel was administered to pigs according to the manufacturer's instructions. Pigs were treated with IL-1ra, saline or Excenel at 24 h, 48 h and one week after challenge with App.

Blood was sampled from pigs by venipuncture at 0 h, 24 h, 48 h, 1 week and 2 weeks after infection. Blood was assayed for immunological parameters as previously described. Pigs were weighed the day prior to challenge, and days 6 and 13 after challenge.

Results

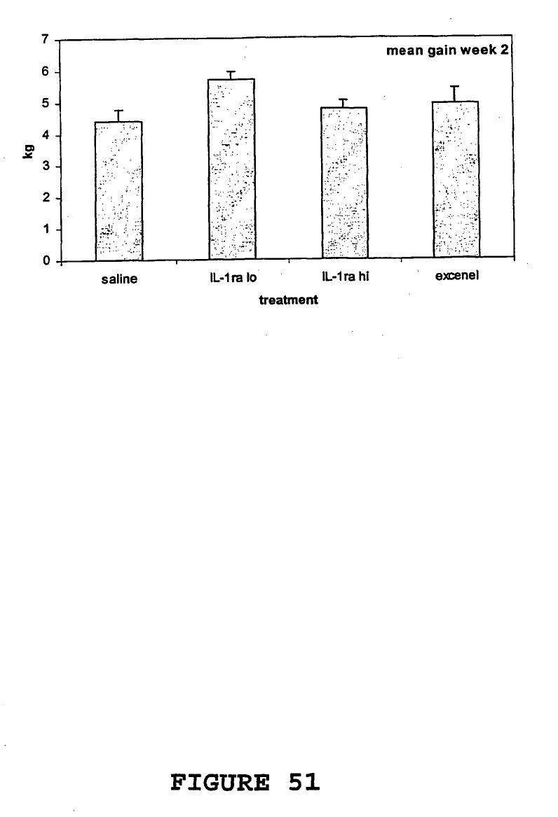

Mean weight gained in the second week after App challenge, illustrated in FIG. 51, shows that pigs treated with low doses of IL-1ra gained more weight than other treatments. Pigs treated with low dose IL-1ra gained on average 5.7 kg in the second week of challenge compared with 4.4 kg weight gain for saline treated controls, and 4.9 kg for antibiotic treatment (FIG. 51).

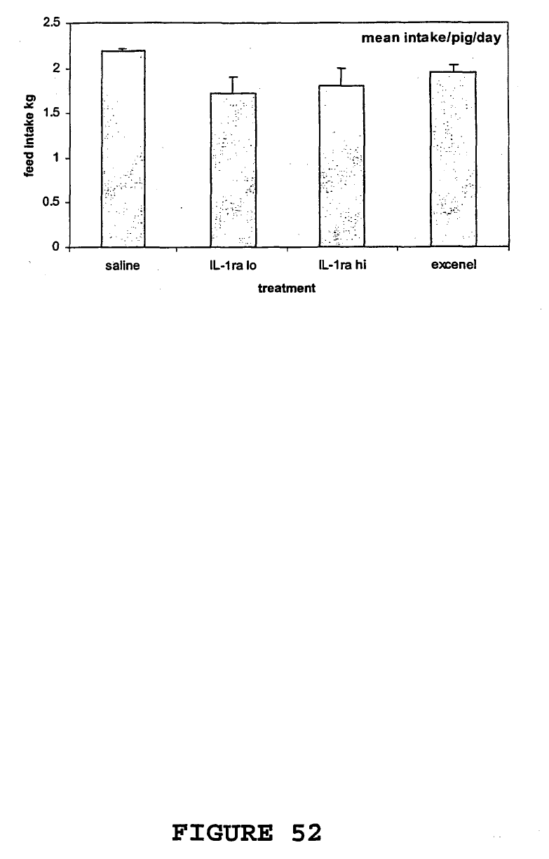

Furthermore, IL-1ra treatment reduced daily feed intake, as did treatment with the antibiotic Excenel compared to saline treatment (FIG. 52). Pigs treated with IL-1ra at low and high doses consumed respectively 1.7 and 1.8 kg of feed per day, while pigs treated with Excenel consumed 2 kg and pigs treated with saline consumed 2.2 kg.

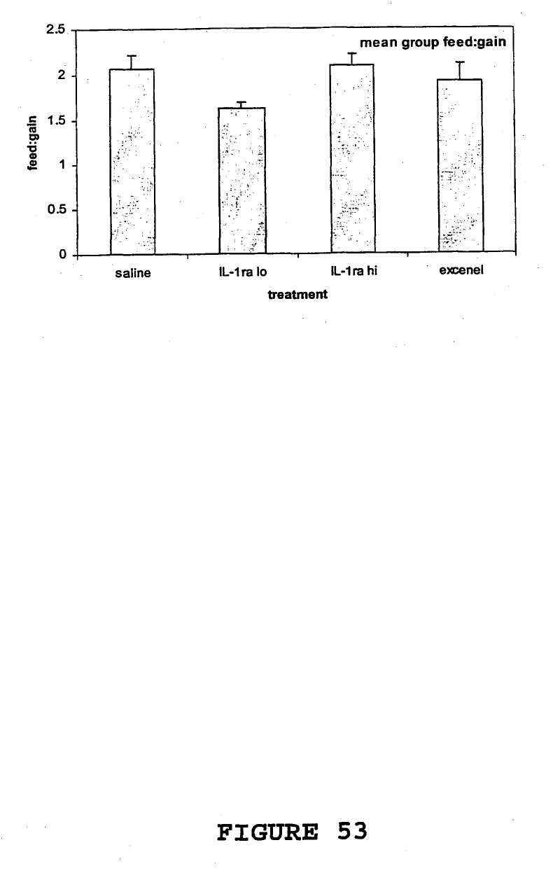

The combined effect of improved weight gain and decreased feed intake resulted in improvements in feed conversion ratio (FCR, feed:gain) for pigs treated with low dose IL-1ra compared to saline controls (FIG. 53). IL-1ra treatment reduced FCR to 1.6 compared to 2.1 for saline treatment.

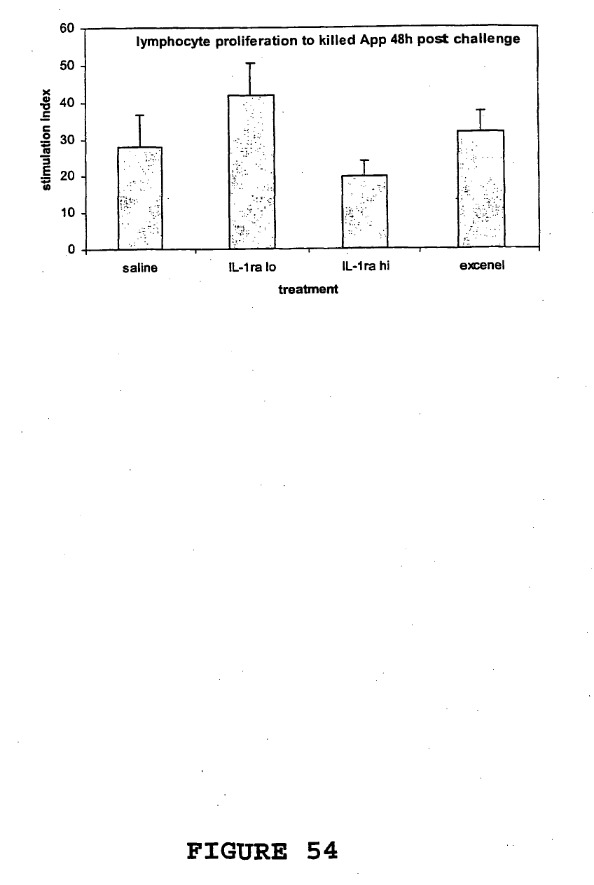

Pigs treated with low dose IL-1ra tended to have improved proliferative responses of lymphocytes in the presence of killed App (FIG. 54). Lymphocyte proliferation assays measure the capacity of lymphocytes to respond to a particular antigen. In this case, the antigen was homologous with the infection and thus, high proliferative responses in vitro are indicative of increased recognition of and mobilisation against the App pathogen in vivo. The trends seen for lymphocyte proliferative responses follow those for FCR—pigs that produced the greatest in vitro response to killed App also had the greatest feed efficiency as evidenced by reduced FCR. Thus, IL-1ra may be improving feed efficiency and weight gain by enhancing specific immune responsiveness.

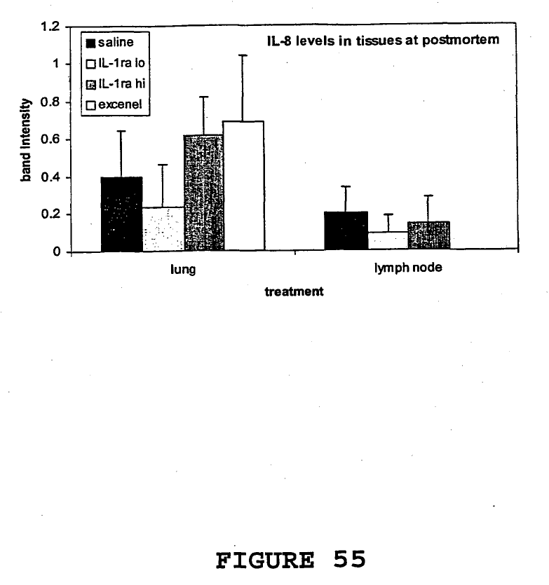

Therapeutic delivery of IL-1ra at low doses to pigs infected wit App also reduced the production of the pro-inflammatory cytokine IL-8 in lung tissue compared to other treatments (FIG. 55). Low dose IL-1ra and Excenel treatment also reduced the production of IL-8 in the caudal-mediastinal lymph nodes, which drain the lungs, compared to saline, treated controls (FIG. 56). Again, these results suggest that IL-1ra is modulating protective immune responses and deleterious inflammatory responses, which may contribute to improved weight gain and feed conversion efficiency in pigs infected with App.

Conclusions

1). IL-1ra applied therapeutically at low doses improved weight gain in pigs infected with App, compared to antibiotic treatment or saline.

2). IL-1ra administered therapeutically decreased feed intake compared to other treatments.

3). Therapeutic administration of IL-1ra greatly improved feed efficiency in pigs infected with App.

4). Therapeutic delivery of IL-1ra in pigs infected with App resulted in enhanced cellular immune responses while diminishing inflammatory responses.

1. A method for improving the growth performance of an animal comprising the step of administering to an animal in need thereof a growth promoting amount of one or more anti-inflammatory agents.

2. A method according to claim 1, wherein the anti-inflammatory agent is administered optionally in combination with a pharmaceutical carrier, adjuvant or vehicle.

3. A method for improving the growth performance of an animal comprising the step of administering to an animal in need thereof a composition comprising an anti-inflammatory agent in conjunction with an antibiotic, optionally in combination with a pharmaceutical carrier, adjuvant or vehicle, wherein said composition achieves a synergistic growth promoting effect.

4. A method for improving the growth performance of an animal comprising the step of administering to an animal in need thereof a compound or composition which increases or supplements endogenous anti-inflammatory agent levels, wherein growth performance is enhanced relative to the growth performance of an animal which has not been administered said compound or composition.

5. A method according to claim 4, wherein the compound or composition is administered prior to, together with, or subsequent to the administration of a growth promoting amount of one or more anti-inflammatory agents.

6. A method according to claim 4 or claim 5, wherein the compound or composition comprises an antagonist of a pro-inflammatory cytokine receptor.

7. A method according to claim 6, wherein the antagonist is of TNF-α receptor, GM-CSF receptor, IL-6 receptor, IL-1 receptor, IL-4 receptor or IL-8 receptor.

8. A method according to any one of claims 4 to 6, wherein the compound or composition comprises IL-10, 1,8-napthosultam substituted compounds or quinoxaline compounds.

9. A method according to claim 4 or claim 5, wherein the compound or composition increases the endogenous level of anti-inflammatory agents by decreasing the amount of pro-inflammatory cytokines.

10. A method according to any one of claims 1 to 3 or 5, wherein the anti-inflammatory agent is a soluble cytokine receptor, cytokine receptor antagonist, cytokine inhibitory factor or biologically active fragment thereof which has an anti-inflammatory effect or an anti-inflammatory agent selected from the group consisting of diclofenac, diflunisal, etodolac, flunix, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic and tolmetin.

11. A method according to claim 10, wherein the soluble cytokine receptor or biologically active fragment thereof is selected from the group consisting of TNFα receptor, IL-6 receptor, IL-1 receptor, IL-4 receptor and IL-8 receptor or a combination thereof that are capable of improving the growth performance of an animal.

12. A method according to claim 10, wherein the soluble cytokine receptor or biologically active fragment thereof is either IL-1 receptor, IL-4 receptor, IL-8 receptor or a combination thereof.

13. A method according to claim 10, wherein the soluble cytokine receptor or biologically active fragment thereof is IL-1 receptor.

14. A method according to claim 10, wherein the cytokine receptor antagonist or biologically active fragment thereof is selected from the group consisting of IL-1ra, IL-6ra, IL-8ra and TNF-αra.

15. A method according to claim 14, wherein the cytokine receptor antagonist or biologically active fragment thereof is IL-1ra.

16. A method according to claim 10, wherein the cytokine inhibitory factor or biologically active fragment thereof is selected from the group consisting of TNF blocking factor and TNF-alpha inhibitor.

17. A method according to any one of claims 1, 2, 4 to 16, further comprising the step of administering an antibiotic.

18. A method according to claim 3, wherein the step of administration the antibiotic is prior to or subsequent to the administration of the anti-inflammatory agent.

19. A method according to claim 3 or claim 17, wherein the antibiotic is selected from the group consisting of amoxycylin, penicillin, procaine, ampicillin, cloxacillin, penicillin G, benzathine, benethamine, ceftiofur, cephalonium, cefuroxime, erythromycin, tylosin, tilmicosin, oleandomycin, kitasamycin, lincomycin, spectinomycin, tetracycline, oxytetracycline, chlortetracycline, neomycin, apramycin, streptomycin, avoparcin, dimetridazole, sulfonamides (including trimethoprim and diaveridine), bacitracin, virginiamycin, monensin, salinomycin, lasalocid, narasin and olaquindox or combinations thereof.

20. A method according to claim 19, wherein the antibiotic is either lincomycin, spectinomycin or amoxicillin or combinations thereof.

21. A method according to any one of claims 1 to 20, wherein the administration is orally, topically, or parenterally.

22. A method according to claim 21, wherein parenteral administration is either by subcutaneous injection, aerosol, intravenous, intramuscular, intrathecal, intrasternal injection, infusion techniques or encapsulated cells.

23. A method according to any one of claims 1 to 22, wherein the administration is either a single dose unit or a multiple dose unit.

24. A method according to any one of claims 1 to 20, wherein the administration is orally as an additive in water and/or feed.

25. A method according to any one of claims 1 to 24, wherein the growth performance of an animal is selected from the group consisting of an increase in growth rate, an increase in efficiency of feed use, an increase in final weight, an increase in dressed weight and decrease in fat content.

26. A method according to any one of claims 1 to 24, wherein the improved growth performance of an animal results from immunoenhancement, anti-parasitic or anti-microbial effects, anti-inflammatory effects or stress reduction.

27. A method according to any one of claims 1 to 26, wherein the animal is either an Artiodactyl or avian.

28. A method according to claim 27, wherein the Artiodactyl is selected from the group consisting of cattle, pigs, sheep, camels, goats and horses.

29. A method according to claim 27, wherein the avian is selected from the group consisting of chickens, turkeys, geese and ducks

30. A method according to claim 27, wherein the animal is cattle, pigs, or sheep.

31. A growth promoting composition comprising one or more anti-inflammatory agents together with one or more pharmaceutical carriers, adjuvants or vehicles.

32. A growth promoting composition according to claim 31, wherein the composition comprises anti-inflammatory agents selected from the group consisting of soluble cytokine receptor, cytokine receptor antagonist, cytokine inhibiting factor or biologically active fragment thereof, diclofenac, diflunisal, etodolac, flunix, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic and tolmetin.

33. A growth promoting composition according to claim 32, wherein the composition comprises one or more soluble cytokine receptor, cytokine receptor antagonist, cytokine inhibitory factor or biologically active fragments thereof and one or more different soluble cytokine receptor, cytokine receptor antagonist, cytokine inhibitory factor or biologically active fragments thereof or one or more different anti-inflammatory agent.

34. A growth promoting composition according to claim 32, wherein the composition comprises one soluble cytokine receptor, cytokine receptor antagonist, cytokine inhibitory factor or biologically active fragment thereof and one different anti-inflammatory agent or a pharmaceutical carrier, adjuvant or vehicle.

35. A growth promoting composition according to claim 32, wherein the soluble cytokine receptor or biologically active fragment thereof is selected from the group consisting of TNFα receptor, IL-6 receptor, IL-1 receptor, IL-4 receptor and IL-8 receptor or a combination thereof.

36. A growth promoting composition according to claim 32, wherein the soluble cytokine receptor or biologically active fragment thereof is either IL-1 receptor, IL-4 receptor, IL-8 receptor or a combination thereof.

37. A growth promoting composition according to claim 32, wherein the soluble cytokine receptor or biologically active fragment thereof is IL-1 receptor.

38. A growth promoting composition according to claim 32, wherein the cytokine receptor antagonist or biologically active fragment thereof is selected from the group consisting of IL-1ra, IL-6ra, IL-8ra and TNF-αra.

39. A growth promoting composition according to claim 32, wherein the cytokine receptor antagonist or biologically active fragment thereof is IL-1ra.

40. A growth promoting composition according to claim 32, wherein the cytokine inhibitory factor or biologically active fragment thereof is selected from the group consisting of TNF blocking factor and TNF-alpha inhibitor.

41. A growth promoting composition according to any one of claims 31 to 40, further comprising one or more antibiotics.

42. A growth promoting composition according to claim 41, wherein the antibiotic is selected from the group consisting of amoxycylin, penicillin, procaine, ampicillin, cloxacillin, penicillin G, benzathine, benethamine, ceftiofur, cephalonium, cefuroxime, erythromycin, tylosin, tilmicosin, oleandomycin, kitasamycin, lincomycin, spectinornycin, tetracycline, oxytetracycline, chlortetracycline, neomycin, apramycin, streptomycin, avoparcin, dimetridazole, sulfonamides (including trimethoprim and diaveridine), bacitracin, virginiamycin, monensin, salinomycin, lasalocid, narasin and olaquindox or combinations thereof.

43. A growth promoting composition according to claim 42, wherein the antibiotic is lincomycin, spectinomycin or amoxicillin or combinations thereof.

44. A method for improving the growth performance of an animal comprising the step of administering to an animal in need thereof a nucleic acid molecule encoding one or more anti-inflammatory agents, wherein the expression of said nucleic acid molecule produces an effective growth promoting amount of one or more anti-inflammatory agents.

45. A method according to claim 44, wherein the nucleic acid molecule is DNA, cDNA, RNA, or a hybrid molecule thereof.

46. A method according to claim 44 or claim 45, wherein the nucleic acid molecule is a full-length molecule or a biologically active fragment thereof.

47. A method according to any one of claims 30 to 32, wherein the nucleic acid molecule is a DNA molecule encoding a soluble cytokine receptor, cytokine receptor antagonist, cytokine inhibitory factor or biologically active fragment thereof.

48. A method according to 47, wherein the DNA encodes a cytokine receptor selected from the group consisting of TNFα receptor, IL-6 receptor, IL-1 receptor, IL-4 receptor and IL-8 receptor or a combination thereof, or a cytokine receptor antagonist selected from the group consisting of IL-1ra, IL-6ra and TNF-αra.

49. A method according to any one of claims 44 to 48, wherein the nucleic acid molecule either integrates into the animal genome or is an extrachromosomal element.

50. A method according to any one of claims 44 to 49, wherein the nucleic acid molecule is administered by injection subcutaneously, intravenously, or intramuscularly or administered as an aerosol.

51. A method according to claim 50, wherein the nucleic acid molecule is administered in an amount of about 1 μg to 2000 μg per dose

52. A method according to claim 50, wherein the nucleic acid molecule is administered in an amount of about 5 μg to 1000 μg per dose.

53. A method according to claim 52, wherein the nucleic acid molecule is administered in an amount of about 6 μg to 200 μg per dose.

54. A method according to any one of claims 44 to 53, wherein the nucleic acid molecule is administered in a vector or as naked DNA.

55. A method according to claim 54, wherein the vector is a porcine adenovirus vector.

56. A construct for delivering in vivo an effective amount of a cytokine receptor, cytokine receptor antagonist, cytokine inhibitory factor or biologically active fragment thereof, comprising:
a) a nucleotide sequence encoding a cytokine receptor, cytokine receptor antagonist, cytokine inhibitory factor or biologically active fragment thereof;
b) a vector comprising a control sequence wherein the control sequence is capable of the controlling the expression of the nucleotide sequence of a) such that a cytokine receptor, cytokine receptor antagonist, cytokine inhibitory factor or biologically active fragment thereof is produced which in turns improves growth performance in an animal.

57. A kit used for improving the growth performance of an animal comprising:
a). one or more anti-inflammatory agents;
b). a delivery device for said anti-inflammatory agents; and
c). instructions for use in the method of the invention.
